(19) United States
(12) Patent Application Publication
(10) Pub. No.: US 2024/0417734 A1
(43) Pub. Date: Dec. 19, 2024

(54) Methods for Treating Glioblastoma by Targeting Oncomirs

(71) Applicants: University of Connecticut, Farmington, CT (US); Yale University, New Haven, CT (US)

(72) Inventors: Raman Bahal, Glastonbury, CT (US); Shipra Malik, Willimantic, CT (US); William Mark Saltzman, New Haven, CT (US); Yazhe Wang, New Haven, CT (US)

(73) Assignees: University of Connecticut, Farmington, CT (US); Yale University, New Haven, CT (US)

(21) Appl. No.: 18/670,162

(22) Filed: May 21, 2024

Related U.S. Application Data
(63) Continuation of application No. PCT/US2022/080356, filed on Nov. 22, 2022.
(60) Provisional application No. 63/282,564, filed on Nov. 23, 2021.

(51) Int. Cl. C12N 15/113; A61K 9/51; A61K 31/495

(52) U.S. Cl. CPC C12N 15/1135; A61K 9/5153; A61K 31/495; C12N 2310/113; C12N 2310/3181

(57) ABSTRACT

The present invention provides cationic polymeric nanoparticles, and nanoparticle formulations thereof. The invention also provides methods for preparing cationic polymeric nanoparticles, and methods of treating diseases, reducing tumor growth, and increasing uptake of a therapeutic agent by a tumor cell in a subject in need thereof.

### RELATED APPLICATIONS

This application is a 35 § U.S.C. 111(a) continuation application which claims the benefit of priority to International Application No. PCT/US2022/080356, filed on Nov. 22, 2022, which, in turn, claims the benefit of priority to U.S. Provisional Application No. 63/282,564, filed on Nov. 23, 2021. The entire contents of each of the foregoing applications are incorporated herein by reference.

### STATEMENT OF GOVERNMENT INTEREST

This invention was made with Government support under CA241194 and CA149128 awarded by the National Institutes of Health. The Government has certain rights in the invention.

### SEQUENCE LISTING

The instant application contains a Sequence Listing which has been submitted electronically in XML format and is hereby incorporated by reference in its entirety. Said XML copy, created on May 14, 2024, is named 098121_00379_SL.xml and is 8,289 bytes in size.

### BACKGROUND OF THE INVENTION

Glioblastoma (GBM) is the most aggressive form of brain tumor, affecting 70-75% of adults with high mortality and median survival of only 14-17 months (A. M. Molinaro, et al., Nat Rev Neurol 15, 405-417 (2019)). GBM has an alarmingly high incidence rate of 4.32 per 100,000 in the United States and a one year survival rate of only 1.4% in patients over 75 years of age (F. G. Davis, et al., Neuro Oncol 22, 301-302 (2020); J. A. Schwartzbaum, et al., Nat Clin Pract Neurol 2, 494-503).

The primary therapeutic approach for GBM is surgical resection followed by radiotherapy and chemotherapy. The primary objective of surgery is to remove as much of the tumor as possible without injuring the surrounding normal brain tissue needed for normal neurological function. However, GBMs are surrounded by a zone of migrating, infiltrating tumor cells that invade surrounding tissues, making it impossible to ever remove the tumor entirely. Surgery provides the ability to reduce the amount of solid tumor tissue within the brain, remove those cells in the center of the tumor that may be resistant to radiation and/or chemotherapy and reduce intracranial pressure. After surgery, when the wound is healed, radiation therapy can begin. The goal of radiation therapy is to selectively kill the remaining tumor cells that have infiltrated the surrounding normal brain tissue. Patients undergoing chemotherapy are administered special drugs designed to kill tumor cells. Chemotherapy with the drug temozolomide is the current standard of treatment for GBM. Temozolomide (TMZ) improves the two-year survival rate from 10.4% to 26.5% in combination with radiotherapy, compared to radiotherapy alone (R. Stupp, et al. N Engl J Med 352, 987-996 (2005)).

Despite significant attention in preclinical and clinical research, glioblastoma (GBM) remains an aggressive disease, with limited survival and poor treatment options. Thus, there is an unmet need for improved therapeutic approaches for targeting molecular genetic mediators of GBM.

### SUMMARY OF THE INVENTION

The inventors of the present invention have also surprisingly discovered that, by packaging these novel sγPNAs into a nanoparticle, for example, a bioadhesive nanoparticle (BNP), the resulting sγPNAs loaded BNPs produced superior anti-miR efficacy in tumor cells and remarkable cytotoxicity when combined with a chemotherapeutic agent, as exemplified by temozolomide (TMZ). Indeed, as set forth in the Examples herein, when delivered in vivo, the sγPNA BNPs dramatically increased the survival in mice with intracranial glioblastoma (GBM). Moreover, the combination of sγPNA BNPs with a chemotherapeutic agent such as temozolomide (TMZ) suppressed tumor growth and significantly improved the survival of GBM mice beyond 120 days with substantial improvement in histopathology. Thus, the inventors of the present invention provide compositions and methods for the treatment of GBM based on tumor specific oncomiRs.

Accordingly, in one aspect, the present invention provides a composition comprising a peptide nucleic acid, wherein the peptide nucleic acid is a cationic gamma-(γ)-modified peptide nucleic acid.

In some embodiments, the peptide nucleic acid comprises a serine modification at the γ-position.

In some embodiments, the peptide nucleic acid comprises at least one, two or three arginine residues on the N-terminus.

In some embodiments, the peptide nucleic acid comprises at least one, two or three arginine residues on the C-terminus.

In some embodiments, the peptide nucleic acid comprises a nucleotide sequence targeting a seed region of an oncomiR.

In some embodiments, the peptide nucleic acid comprises from N-terminus to C-terminus: three arginine residues, the nucleotide sequence targeting the seed region of the oncomiR, and one arginine at the C-terminus.

In some embodiments, the oncomiR is selected from the group consisting of miR-21 and miR-10b.

In some embodiments, the composition comprises a peptide nucleic acid comprising a nucleotide sequence targeting the seed region of miR-21.

In some embodiments, the composition comprises a peptide nucleic acid comprising a nucleotide sequence targeting the seed region of miR-10b.

In some embodiments, the composition comprises a peptide nucleic acid comprising a nucleotide sequence targeting the seed region of miR-21, and a peptide nucleic acid comprising a nucleotide sequence targeting the seed region of miR-10b.

In some embodiments, the nucleotide sequence targeting the seed region of the oncomiR comprises about 5, 6, 7, or 8 nucleotides in length.

In some embodiments, the nucleotide sequence targeting the seed region of the oncomiR comprises about 8 nucleotides in length.

In some embodiments, the nucleotide sequence targeting the seed region of miR-21 comprises GATAAGCT.

In some embodiments, the nucleotide sequence targeting the seed region of miR-10b comprises TACAGGGT.

In one aspect, the present invention provides a nanoparticle comprising any of the peptide nucleic acids of the invention, wherein the peptide nucleic acid is encapsulated within the nanoparticle, and wherein the peptide nucleic acid is a cationic gamma-(γ)-modified peptide nucleic acid.

In some embodiments, the nanoparticle comprises a poly-lactic acid and hyperbranched polyglycerol (PLA-HPG) polymer.

In some embodiments, the nanoparticle is modified with aldehyde groups.

In some embodiments, the nanoparticle is bioadhesive.

In some embodiments, the nanoparticle comprises a first peptide nucleic acid comprising a nucleotide sequence targeting a seed region of miR-21, and a second peptide nucleic acid comprising a nucleotide sequence targeting a seed region of miR-10b, wherein the first and the second peptide nucleic acids are encapsulated within the nanoparticle, wherein the first and the second peptide nucleic acids are each cationic gamma-(γ)-modified peptide nucleic acids.

In one aspect, the present invention provides a pharmaceutical composition comprising any of the compositions of the invention, or any of the nanoparticles of the invention, and a pharmaceutically acceptable excipient.

In some embodiments, the pharmaceutical composition comprises a first nanoparticle comprising a first peptide nucleic acid comprising a nucleotide sequence targeting a seed region of miR-21 and a second nanoparticle comprising a second peptide nucleic acid comprising a nucleotide sequence targeting a seed region of miR-10b.

In one aspect, the present invention provides a method of treating a disease in a subject in need thereof, comprising administering to the subject a therapeutically effective amount of any of the compositions of the invention, any of the nanoparticles of the invention, or any of the pharmaceutical compositions of the invention, thereby treating the disease in the subject in need thereof.

In some embodiments, the disease is cancer.

In some embodiments, the cancer is glioblastoma.

In another aspect, the present invention provides a method of reducing a tumor growth in a subject in need thereof, comprising administering to the subject a therapeutically effective amount of any of the compositions of the invention, any of the nanoparticles of the invention, or any of the pharmaceutical compositions of the invention, thereby reducing the tumor growth in the subject in need thereof.

In one aspect, the present invention provides a method of prolonging survival time of a subject in need thereof, comprising administering to the subject a therapeutically effective amount of any of the compositions of the invention, any of the nanoparticles of the invention, or any of the pharmaceutical compositions of the invention, thereby prolonging survival time of the subject in need thereof.

In one aspect, the present invention provides a method of increasing sensitivity to a chemotherapy in a subject in need thereof, comprising administering to the subject a therapeutically effective amount of any of the compositions of the invention, any of the nanoparticles of the invention, or any of the pharmaceutical compositions of the invention, thereby increasing sensitivity to the chemotherapy in the subject in need thereof.

In one aspect, the present invention provides a method of increasing apoptosis of tumor cells in a subject in need thereof, comprising administering to the subject a therapeutically effective amount of any of the compositions of the invention, any of the nanoparticles of the invention, or any of the pharmaceutical compositions of the invention, thereby increasing apoptosis of the tumor cells in the subject in need thereof.

In some embodiments, the method results in a decrease in miR-10b and/or miR21 levels.

In some embodiments, the method results in a decrease in VEGFA and ITGB8 levels.

In some embodiments, the composition, the nanoparticle or the pharmaceutical composition is administered to the subject by convection-enhanced drug delivery (CED).

In some embodiments, the subject is a human subject.

In some embodiments, the methods further comprises administering to the subject an additional therapeutic agent.

In some embodiments, the additional therapeutic agent is selected from the group consisting of a chemotherapeutic agent, a growth inhibitory agent, an anti-angiogenesis agent, an anti-neoplastic composition and a combination of any of the foregoing.

In some embodiments, the chemotherapeutic agent is temozolomide.

The present invention is illustrated by the following drawings and detailed description, which do not limit the scope of the invention described in the claims.

### BRIEF DESCRIPTION OF DRAWINGS

FIGS. 1A-1C depict the sequence of peptide nucleic acids (PNAs) used in the study. FIG. 1A depicts the chemical structures of DNA, PNA and serine-γ-PNA. FIG. 1B depicts the sequence of oncomiR-21 (SEQ ID NO:1) and oncomiR-10b (SEQ ID NO:2), where the underlined segment is the seed region. FIG. 1C depicts the PNAs used in this study. sγPNA-21 and sγPNA-10b were serine-γPNAs designed to bind the seed region of oncomiR-21 and oncomiR-10b, respectively. Three arginine (RRR) residues were appended to the N-terminus and one arginine (R) on the C-terminus. Scr-sγPNA-21 and Scr-sγPNA-10b were scrambled versions of sγPNA-21 and sγPNA-10b, respectively. PNA-21 (SEQ ID NO:3) and PNA-10b (SEQ ID NO:4) were regular PNAs designed to bind full length oncomiR-21 and oncomiR-10b, respectively. PNAs were conjugated with 5-carboxytetramethylrhodamine (TAMRA), a fluorescent dye for imaging. 000 represents 8-amino-2,6,10-trioxaoctanoic acid residues (Mini-PEG). These were present to form a flexible linker connecting the TAMRA and Watson Crick binding regions of the PNAs.

FIGS. 2A-2E depict the characterization of sγPNA loaded nanoparticles. FIG. 2A depicts the TEM images of sγPNA/NNP and sγPNA/BNP. Scale bar, 100 nm. FIG. 2B depicts the size stability of sγPNA/NNP and sγPNA/BNP in aCSF. FIG. 2C depicts the amount of sγPNA (sγPNA-21 and sγPNA-10b) released from NPs over time during incubation in buffered saline quantified as a percentage of amount loaded. The data were shown as mean±SD (n=3). FIG. 2D depicts cellular uptake analyzed by flow cytometry. Top to bottom: U87 cells treated with γPNA/BNP, γPN/NNP, free γPNA (physical mixture of free γPNA1 and γPNA2) and untreated cell (control). FIG. 2E depicts confocal microscopic images of cells treated by free γPNA or γPNA loaded NPs. PNAs were conjugated with TAMRA (red), F-actin was labelled with phalloidin (green) and nucleus was stained with Hoechst (blue). Scale bar, 20 μm.

FIGS. 3A-3F depict that sγPNA mediated simultaneous knockdown of miR-21 and miR-10b induced apoptosis in glioma (U87) cells. FIG. 3A depicts the expression analysis of miR-10b and miR-21 levels in U87 cells after treatment with Scr-sγPNA/NNP, Scr-sγPNA/BNP, sγPNA/NNP, sγPNA/BNP, and free sγPNA. Scr-sγPNA/NNP and Scr-sγPNA/BNP indicates the physical mixture of Scr-sγPNA-21 and Scr-sγPNA-10b loaded NPs (NNP or BNP). sγPNA/NNP and sγPNA/BNP indicates the physical mixture of sγPNA-21 and sγPNA-10b loaded NPs (NNP or BNP). Data were expressed as mean±SD (n=3). FIG. 3B depicts the relative miRNA level in U87 cells treated with different formulations. PNA/BNP was the physical mixture of PNA-21 loaded BNP and PNA-10b loaded BNP. FIG. 3C depicts the percentage of early apoptotic cells after different treatments with sγPNA/NNP and BNP with or without temozolomide (TMZ). Data were expressed as mean±SD (n=3). FIG. 3D depicts caspase 3 and caspase 7 activities in cells treated by combinations of NPs and TMZ for 72 h. Data were expressed as mean±SD (n=3). FIG. 3E depicts the viability of cells treated with increasing doses of TMZ, with or without sγPNA NPs (NNP or BNP) treatment. FIG. 3F depicts the viability of cells treated with different doses of NPs for 72 h.

FIGS. 4A-4E depict the RNA sequencing analysis of sγPNA/BNP treated U87 cells. FIG. 4A depicts the hierarchal clustering analysis of differentially expressed genes (DEGs) in U87 cells treated with sγPNA/BNP in comparison to control (untreated U87 cells). FIG. 4B depicts the gene ontology analysis of differentially expressed downregulated genes. FIG. 4C depicts the heatmap of DEGs associated with PI3-Akt pathway. FIG. 4D depicts the heatmap of DEGs associated with the focal adhesion pathway. FIG. 4E depicts the heatmap of DEGs associated with the HIF-1 pathway. sγPNA/BNP indicates the physical mixture of sγPNA-21 and sγPNA-10b loaded BNP.

FIGS. 5A-5E depict the identification of downstream targets after simultaneous knockdown of miR-21 and miR-10b. FIG. 5A is a Venn diagram depicting the correlation between predicted miR-10b and miR-21 targets with downregulated DEGs in sγPNA/BNP treated U87 cells. FIGS. 5B-5D are Venn diagrams depicting the correlation between predicted miR-10b and miR-21 targets with downregulated DEGs in sγPNA/BNP treated U87 cells and genes associated with the PI3-Akt pathway (FIG. 5B), the HIF-1 signaling pathway (FIG. 5C), and the focal adhesion pathway (FIG. 5D). FIG. 5E depicts the gene expression analysis of VEGFA and ITGB8 in sγPNA/BNP treated U87 cells. sγPNA/BNP indicates the physical mixture of sγPNA-21 and sγPNA-10b loaded BNP.

FIGS. 6A-6J depict that the combination of sγPNA/BNP and Temozolomide (TMZ) improved the survival in an orthotopic mice model of glioblastoma. FIG. 6A depicts the biodistribution of BNP containing sγPNA-TAMRA on day 0, 1, 3, 7 and 14 after convention enhanced delivery (CED) via IVIS and microscopic imaging. Scale bar, 200 μm. FIG. 6B depicts the survival of mice bearing U87 derived intracranial gliomas after treatment with sγPNA/BNP, TMZ and combination treatment of sγPNA/BNP with TMZ. NPs were administered by CED at 200 mg/ml dose and TMZ was administered intraperitoneally at 25 mg/kg dose. FIG. 6C depicts the H&E staining of control and sγPNA/BNP+TMZ treated mice brain at the end of the survival study. Scale bar, 50 μm. FIG. 6D depicts the levels of miR-21 and miR-10b in gliomas of control and sγPNA/BNP+TMZ treated mice at the end of survival study. The levels of miR-10 and miR-21 were at similar levels as contralateral (to injection site) brain region and healthy mice brain. Results were represented as mean±SD (n=3 biologically independent animals). FIG. 6E depicts the gene expression levels of miR-10b and miR-21 in mice gliomas 48 h after CED of sγPNA/BNP on day 14. FIG. 6F depicts the gene expression levels of miR-10b and miR-21 in mice gliomas 48 h after CED of sγPNA/BNP on day 28. FIG. 6G depicts the levels of VEGFA and ITGB8 in tumors 48 h post CED on day 14. FIG. 6H depicts the levels of VEGFA and ITGB8 in tumors 48 h post CED on day 28. Results were represented as mean±SD (n=3 biologically independent animals). FIG. 6I depicts the clinical chemistry of blood samples including white blood count (WBC), platelets (PLT), red blood cells (RBCs) from mice after 48 h of administration of sγPNA/BNP and sγPNA/BNP+TMZ. FIG. 6J depicts the blood biochemistry including blood urea nitrogen (BUN), alanine aminotransferase (ALT), and aspartate transaminase (AST) from mice after 48 h of administration of sγPNA/BNP and sγPNA/BNP+TMZ. Results were represented as mean±SD (n=3 biologically independent animals).

FIGS. 7A-7J depict the efficacy of targeting oncomiR-10b and 21 via BNP delivered sγPNAs in patient derived xenograft (PDX) mice model of glioblastoma. FIG. 7A depicts the confocal microscopic images of G22 cells treated by free sγPNA or sγPNA loaded NPs. PNAs were conjugated with TAMRA (red), F-actin was labelled with phalloidin (green) and nucleus was stained with Hoechst (blue). Scale bar, 25 μm. FIG. 7B depicts the survival of mice bearing patient (G22) derived intracranial gliomas after treatment with BNPs containing sγPNA, TMZ and combination treatment of BNP with TMZ. BNPs were administered by convention enhanced delivery (CED) at 200 mg/ml dose and TMZ was administered intraperitoneally at 25 mg/kg dose. FIG. 7C depicts the histology of H&E-stained control and NP+TMZ treated mice brain at the end of the survival study. Scale bar represents 75 μm. FIG. 7D depicts the levels of miR-21 and miR-10b in gliomas of control and BNP+TMZ treated mice at end of survival study. The levels of miR-10 and miR-21 were at similar levels as contralateral (to injection site) brain region and healthy mice brain. Results were represented as mean+/−SD (n=3 biologically independent animals). FIG. 7E depicts the gene expression levels of miR-10b and miR-21 in mice gliomas 48 h after CED of NPs on day 14 post tumor implantation. Results were represented as mean+/−sem (n=3 biologically independent animals). FIG. 7F depicts the gene expression levels of miR-10b and miR-21 in mice gliomas 48 h after CED of NPs on day 28 post tumor implantation. Results were represented as mean+/−sem (n=3 biologically independent animals). FIG. 7G depicts the levels of downstream targets VEGFA and ITGB8 in mice gliomas 48 h after CED of NPs on day 14. Results were represented as mean+/−sem (n=3 biologically independent animals). FIG. 7H depicts the levels of downstream targets VEGFA and ITGB8 in mice gliomas 48 h after CED of NPs on day 28. Results were represented as mean+/−sem (n=3 biologically independent animals). FIG. 7I depicts the clinical chemistry of blood samples including white blood count (WBC), platelets (PLT), red blood cells (RBCs) from mice after 48 h of administration of BNP and BNP+TMZ. FIG. 7J depicts the blood biochemistry including blood urea nitrogen (BUN), alanine aminotransferase (ALT), and aspartate transaminase (AST) from mice after 48 h of administration of BNP and BNP+TMZ. Results were represented as mean+/−sem (n=3 biologically independent animals).

FIG. 8 depicts the HPLC chromatograms of the PNAs used in the study.

FIGS. 9A-9B depict the gel shift assay of PNAs with miR-21 and miR-10b. FIG. 9A depicts the gel-shift assay of miR-21 and miR 10b targets with sγPNA-21 and sγPNA-10b, respectively at PNA:miR ratio of 2:1. FIG. 9B depicts the gel-shift assay of miR-21 and miR 10b target with sγPNA-21 and sγPNA-10b, respectively at PNA:miR ratio of 4:1. PNAs were incubated with the target miRs in simulated physiological buffer conditions (10 mM NaPi, 150 mM KCl and 2 mM MgCl2) for 16 hours at 37° C. followed by non-denaturing PAGE separation and SYBR Gold staining.

FIG. 10 depicts the gel-shift assay of sγPNA-21 and sγPNA-10b with the target miR 21 and miR 10b as well as mixture of both the target miRs. PNAs were incubated with the target miRs at PNA:miR ratio of 1:1 in simulated physiological buffer conditions (10 mM NaPi, 150 mM KCl and 2 mM MgCl2) for 16 hours at 37° C. followed by non-denaturing PAGE separation and SYBR Gold staining.

FIG. 11 depicts the quantification of bio-adhesiveness of sγPNA loaded NNPs and BNPs using poly-l-lysine (PLL) coated glass. NPs were incubated with PLL glass cover slides for 30 min then washed with water. Acetonitrile was added to each slide to harvest the remaining sγPNA-loaded NPs. The concentration of remaining PNA on the slides was quantified using the methods described. Data was normalized to unwashed controls. ***p<0.001 vs NNP.

FIGS. 12A-12B depict the cellular uptake of sγPNA NNP/BNP in glioma cells. FIG. 12A depicts the quantification of cellular uptake in U87 cells by flow cytometry analysis. ***p<0.001. FIG. 12B depicts the confocal microscopic images of LN229 cells treated by sγPNA loaded nanoparticles. PNAs were conjugated with TAMRA (red), F-actin was labelled with phalloidin (green) and nucleus was stained with Hoechst (blue). Scale bar, 20 μm.

FIGS. 13A-13B depict gene expression of PTEN, miR-21, and miR-10b. FIG. 13A depicts the relative PTEN mRNA level in U87 cells transfected with sγPNA NNP and sγPNA BNP. ***p<0.001 vs control, **p<0.01 vs control and *p<0.05 vs control. Data were expressed as mean±SD (n=3). FIG. 13B depicts the qRT-PCR analysis of relative miR-10b and miR-21 level in cells treated by sγPNA-21 BNP, sγPNA-10b BNP and SγPNA BNP nanoparticles. sγPNA BNP nanoparticles were physical mixture of sγPNA-21 BNP and sγPNA-10b BNP. NNP indicates PLA-HPG nanoparticles and BNP indicates PLA-HPG-CHO nanoparticles. **p<0.001 vs control.

FIG. 14 depicts the flow cytometry analysis of cell apoptosis after various treatments using Annexin V assay.

FIGS. 15A-15F depict that sγPNA NNP/BNP induced apoptosis in U87 cells. FIG. 15A depicts the quantification of apoptotic cells treated by PNA/BNP, sγPNA/BNP with and without TMZ by flow cytometry. *p<0.05, **p<0.01. FIG. 15B depicts caspase 3 and caspase 7 activities of cells treated by various formulations for 48 h. *p<0.05. FIG. 15C depicts cell viability of U87 cells treated by increasing doses of sγPNA/NNP and sγPNA/BNP for 48 h. FIG. 15D depicts cell viability of tumor cells (U87) and human astrocytes (HAc) treated by sγPNA/BNP and increasing doses of TMZ for 72 h. sγPNA/BNP were physical mixture of sγPNA-21 BNP and sγPNA-10b BNP. PNA/BNP were physical mixture of PNA-21 BNP and PNA-10b BNP. NNP indicates PLA-HPG nanoparticles and BNP indicates PLA-HPG-CHO nanoparticles. FIG. 15E depicts the flow cytometry analysis induced by free γPNA, TMZ, γPNA/PLA-HPG, γPNA/PLA-HPG-CHO, γPNA/PLA-HPG with TMZ, γPNA/PLA-HPG-CHO with TMZ using Annexin V assay. PE-Annexin V positive and 7AAD-negative populations were identified as early apoptotic cells, PE-Annexin V positive and 7AAD-postive populations were identified as late apoptotic/necrotic cells, PE-Annexin V negative and 7AAD-negative populations were identified as live cells. FIG. 15F depicts the percentage of early apoptotic cells following different treatments. Data were expressed as mean±SD (n=3). ***p<0.001.

FIG. 16 depicts the RNA sequencing work flow. U87 cells were treated with BNP containing sγPNA-10b or sγPNA-21 or combination of BNP loaded with sγPNA-21 and sγPNA-10b for 72 hours. Total RNA was collected from the treated and untreated (control) cells for RNA sequencing analysis.

FIG. 17 depicts the differentially expressed genes after sγPNA BNP mediated knockdown of both miR-10b and miR-21. The volcano plot showing differentially upregulated (left) and downregulated (right) genes in U87 cells treated with sγPNA/BNP (physical mixture of sγPNA-21/BNP and sγPNA-10b/BNP) for 72 h when compared against the untreated cells (Control).

FIGS. 18A-18C depict RNA sequencing analysis after sγPNA-21 BNP mediated knockdown of miR-21 in U87 cells. FIG. 18A is a heatmap showing the hierarchical clustering of differentially expressed genes in U87 cells treated with sγPNA-21/BNP in comparison to the control (untreated). FIG. 18B is a volcano plot showing differentially expressed genes in sγPNA-21/BNP treated U87 cells. FIG. 18C depicts the gene ontology (GO) enrichment analysis of the differentially expressed downregulated genes in U87 cells treated with sγPNA-21/BNP in comparison to the control (untreated cells).

FIGS. 19A-19C depict RNA sequencing analysis after sγPNA-10b BNP mediated knockdown of miR-10b in U87 cells. FIG. 19A is a heatmap showing the hierarchical clustering of differentially expressed genes in U87 cells treated with sγPNA-10b/BNP in comparison to the control (untreated). FIG. 19B is a volcano plot showing differentially expressed genes in sγPNA-10b/BNP treated U87 cells. FIG. 19C depicts the gene ontology (GO) enrichment analysis of the differentially expressed downregulated genes in U87 cells treated with sγPNA-10b/BNP in comparison to the control (untreated cells).

FIGS. 20A-20B depict heatmaps of PI3-Akt and focal adhesion pathway after miR-21 knockdown in U87 cells showing that the differentially expressed downregulated genes in U87 cells treated with sγPNA-21/BNP were associated with PI3-Akt pathway (FIG. 20A), and focal adhesion pathway (FIG. 20B).

FIG. 21 depicts a heatmap of PI3-Akt pathway after miR-10b knockdown in U87 cells showing that the differentially expressed downregulated genes in U87 cells treated with sγPNA-10b/BNP were associated with PI3-Akt pathway.

FIGS. 22A-22B depict the correlation of survival probabilities and levels of miR-10b or miR-21. FIG. 22A depicts the survival probability of GBM patients in association with the levels of miR-10b. FIG. 22B depicts the survival probability of GBM patients in association with the levels of miR-21.

FIG. 23 depicts the correlation of survival probabilities of GMB patients and levels of both miR-10b and miR-21.

FIG. 24 depicts immunostaining of Ki67 in TAMRA-sγPNA/BNP treated mice (CED) after one day. Blue indicates nucleus, green indicates Ki67, orange indicates TAMRA-sγPNA. Scale bar in top panel is 150 μm and bottom panel is 75 μm.

FIGS. 25A-25B depict mean fluorescence intensity of TAMRA-sγPNA in the brain. FIG. 25A depicts the mean fluorescence intensity of TAMRA-sγPNA in the brain at different time points and FIG. 25B depicts the percentage of day 0. Data were expressed as mean±sem, n=3.

FIG. 26 depicts the survival study of animals treated with lower dose of TMZ (12.5 mg/kg) and combination treatment with TMZ and sγPNA/BNP.

FIG. 27 depict the Ki67 staining of control and sγPNA/BNP+TMZ treated U87 tumor-bearing mice brain at the end of the survival study. Scale bar, 75 μm.

FIG. 28 depicts the in vivo toxicity analysis of BNP and BNP+Temozolomide (TMZ). The complete blood count analysis of blood samples from mice treated with BNP and BNP+TMZ after 48 hours of treatment. Results were represented as mean±sem (n=3 mice/group). HCT: Hematocrit, HGB: Hemoglobin, MCV: Mean Corpuscular Volume, MCH: Mean Corpuscular Hemoglobin, MCHC: Mean Corpuscular Hemoglobin Concentration, RDW-SD: Red Cell Distribution Width, MPV: Mean Platelets Volume.

FIG. 29 depicts the biochemistry analysis of blood samples from mice treated with BNP and BNP+TMZ after 48 hours of treatment. Results were represented as mean±sem (n=3 mice/group). LDH: Lactate dehydrogenase.

FIG. 30 depicts H&E staining of major organs including heart, liver, spleen, lung, liver, and kidney in all groups of mice from survival study. Scale bar, 75 μm.

FIGS. 31A-31C depict cellular uptake and efficacy of sγPNA in G22 cells (patient derived glioblastoma cells). FIG. 31A depicts quantification of cellular uptake of sγPNA in G22 cells via flow cytometry analysis. FIG. 31B depicts the levels of miR-10b and miR-10b in G22 cells (patient derived glioblastoma cells) treated in vitro with sγPNA (sγPNA-10b+sγPNA-21) and BNPs containing sγPNAs and scr-sγPNAs. FIG. 31C depicts cell viability of G22 patient derived glioma cells treated by BNP and increasing doses of TMZ for 72 h. BNP were physical mixture of sγPNA-21 BNP and sγPNA-10b BNP. BNP indicates PLA-HPG-CHO nanoparticles.

FIG. 32 depicts Ki67 staining of control and sγPNA/BNP+TMZ treated G22 tumor-bearing mice brain at the end of the survival study. Scale bar, 75 μm.

FIG. 33 depicts the complete blood count analysis of blood samples from G22 PDX mice model treated with BNP and BNP+TMZ after 48 hours of treatment. Results were represented as mean±sem (n=4 mice/group). HCT: Hematocrit, HGB: Hemoglobin, MCV: Mean Corpuscular Volume, MCH: Mean Corpuscular Hemoglobin, MCHC: Mean Corpuscular Hemoglobin Concentration, RDW-SD: Red Cell Distribution Width, MPV: Mean Platelets Volume.

FIG. 34 depicts the biochemistry analysis of blood samples from G22 PDX mice model treated with BNP and BNP+TMZ after 48 hours of treatment. Results were represented as mean±sem (n=3 mice/group). LDH: Lactate dehydrogenase.

FIG. 35 depicts H&E staining of major organs including heart, liver, spleen, lung, liver, and kidney in all groups of mice from survival study in G22 PDX mice model of glioblastoma. Scale bar, 75 μm.

FIG. 36 is a schematic showing the overall study design.

### DETAILED DESCRIPTION

The present invention is based, at least in part, on the generation of novel short gamma-(γ)-modified peptide nucleic acids (sγPNA) which specifically target the seed region of an oncomiR associated with glioblastoma (GBM) invasiveness and progression, e.g., miR21 and/or miR10b, with high affinity.

The inventors of the present invention have also surprisingly discovered that, by packaging these novel sγPNAs into a nanoparticle, for example, a bioadhesive nanoparticle (BNP), the resulting sγPNAs loaded BNPs produced superior anti-miR efficacy in tumor cells and remarkable cytotoxicity when combined with a chemotherapeutic agent, as exemplified by temozolomide (TMZ). Indeed, as set forth in the Examples herein, when delivered in vivo, the sγPNA BNPs dramatically increased the survival in mice with intracranial glioblastoma (GBM). Moreover, the combination of sγPNA BNPs with a chemotherapeutic agent such as temozolomide (TMZ) suppressed tumor growth and significantly improved the survival of GBM mice beyond 120 days with substantial improvement in histopathology. Thus, the inventors of the present invention provide compositions and methods for the treatment of GBM based on tumor specific oncomiRs.

### I. DEFINITIONS

As used herein, each of the following terms has the meaning associated with it in this section.

The articles “a” and “an” are used herein to refer to one or to more than one (i.e., to at least one) of the grammatical object of the article. By way of example, “an element” means one element or more than one element, e.g., a plurality of elements.

The term “including” is used herein to mean, and is used interchangeably with, the phrase “including but not limited to”.

The term “or” is used herein to mean, and is used interchangeably with, the term “and/or,” unless context clearly indicates otherwise.

As used herein, the terms “peptide nucleic acid”, “PNA”, and “PNAs”, are used interchangeably and refer to synthetic DNA analogs in which the phosphodiester backbone is replaced by repetitive units of uncharged N-(2-aminoethyl) glycine. Purine or pyrimidine bases are linked to the nitrogen of the N-(2-aminoethyl)-glycine backbone units via a methyl carbonyl linker. PNAs exhibit many interesting properties, including high binding affinity to DNA and RNA, a low dependency on ionic strength, high chemical stability, high sequence specificity, and resistance to both nucleases and proteases.

As used herein, the term “gamma-(γ)-modified peptide nucleic acid” or “γPNA” refers to a backbone-modified PNA that possesses a stereogenic center through modifications introduced at gamma (γ)-carbon of the backbone. Specifically, a simple modification of introducing a L-serine side chain at the gamma position of N-(2-aminoethyl) glycine backbone of PNA caused the single-stranded molecule to assume a pre-organized helical arrangement. Due to the stereogenic center and serine side chain, the gamma-PNA itself forms an alpha-helical structure, thereby reducing the self-aggregation, improving the solubility and forming a more stable duplex with the target DNA and RNA. These features provide higher binding affinity to the target. In addition, various modifications such as internal multi-labeling are possible at any gamma (γ)-position.

As used herein, the terms “microRNA”, “miRNA” or “miR” are used interchangeably and refer to short, non-coding RNAs (about 20-25 nucleotide in length) that regulate gene expression at the post-transcriptional level. The dysregulation of miRs, either upregulation (where they are known as oncomiRs) or downregulation, plays an important role in several malignancies. Previous studies have reported aberrant miR expression levels in GBM patients; some of these are associated with poor prognosis and low overall survival.

As used herein, the term “oncomiR” refers to microRNAs that typically target a tumor suppressor, which are generally upregulated in different types of cancer. OncomiRs are known to promote tumorigenesis by inhibiting tumor suppressor genes and/or genes responsible for apoptosis. Conversely, miRNAs, which downregulate oncogenes, are defined as tumor suppressor miRs. Tumor suppressor miRs are often lost in cancer and this usually happens through multiple mechanisms including promoter methylation, mutation or deletion, or defective miRNA processing.

In particular embodiments, the oncomiRs targeted by the compositions and methods of the present invention include miR-10b and/or miR-21, which appear to be the most highly upregulated oncomiRs contributing to GBM. miR-10b enhances GBM growth by negatively regulating Bim (BCL2 interacting mediator of death), TFAP2C (transcription factor AP-2γ), CDKN2A/16 (tumor suppressor), and p21 (cell cycle inhibitor) expression. Inhibiting miR-10b reduces the growth of intracranial GBM tumors in animal models. Similarly, upregulated miR-21 levels increase GBM invasiveness by inhibiting matrix metalloproteinase (MMP), increase proliferation via negative regulation of insulin-like growth factor binding protein-3 (IGFBP3) or phosphatase and tensin homolog (PTEN), and promote tumor stemness via SOX-2, a transcription factor. Current therapeutic strategies, which are focused on targeting a single oncomiR, have shown limited efficacy against GBM.

As used herein, the term “seed region” refers to a contiguous sequence of at least 6 nucleotides, e.g., 6, 7, or 8 nucleotides, beginning at position two from the miRNA 5′-end. In some embodiments, the seed region is mostly situated at positions 2-7 from the miRNA 5′-end. In some embodiments, the seed region is mostly situated at positions 2-8 from the miRNA 5′-end. In some embodiments, the seed region is mostly situated at positions 2-9 from the miRNA 5′-end. Even though base pairing of miRNA and its target does not match perfect, the seed sequence has to be perfectly complementary.

As used herein, the term “nanoparticle” and “NP” are used interchangeably and refer to any particle having a diameter of less than 1000 nanometers (nm). In some embodiments, a nanoparticle has a diameter of less than 300 nm, as defined by the National Science Foundation. In some embodiments, a nanoparticle has a diameter of less than 100 nm as defined by the National Institutes of Health. In some embodiments, the nanoparticles are made of a block copolymer of poly(lactic acid) and hyperbranched polyglycerol (PLA-HPG). In some embodiments, the nanoparticles are functionalized with aldehyde groups, creating PLA-HPG-CHO. Nanoparticles formed from PLA-HPG-CHO are bioadhesive.

As used herein, the term “bioadhesive nanoparticle” and “BNP” are used interchangeably and refer to any nanoparticles with adhesive properties, for examples, nanoparticles with surface modifications that can enhance the association of nanoparticles with particular cell populations, such as tumor cells. In some embodiments, the BNPs are produced by oxidation of the nonadhesive nanoparticles, e.g., nanoparticles made of a block copolymer of poly(lactic acid) and hyperbranched polyglycerol (PLA-HPG), which result in conversion of vicinal diols on the surface of the nonadhesive nanoparticles into aldehydes on the BNPs, e.g., creating PLA-HPG-CHO. Aldehydes spontaneously react with proteins to form a variety of bonds, such as Schiff-base bonds; therefore, the presence of surface aldehydes on BNPs leads to particle adhesion to protein-rich materials. In some embodiments, NPs formed from PLA-HPG-CHO are bioadhesive. In some embodiments, successful conversion to the bioadhesive state can be confirmed by measuring NP adhesion to poly(L-lysine) coated glass.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, partial or complete alleviation of symptoms, diminishment of any direct or indirect pathological consequences of the disease, preventing metastasis, increasing/extending overall survival, decreasing the rate of disease progression, amelioration or palliation of the disease state, and remission or improved prognosis. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

As used herein, the term “administering” to a subject includes dispensing, delivering or applying a composition of the disclosure to a subject by any suitable route for delivery of the composition to the desired location in the subject. Alternatively, or in combination, delivery is by the topical, parenteral or oral route, intracerebral injection, intrathecal injection, intramuscular injection, subcutaneous/intradermal injection, intravenous injection, buccal administration, transdermal delivery and administration by the rectal, colonic, vaginal, intranasal or respiratory tract route. In some embodiments, the compositions are administered by convection-enhanced drug delivery (CED) to the brain.

As used herein, the term “convection enhanced delivery” or “CED’ refers to a therapeutic strategy to facilitate targeted delivery of pharmaceuticals to the brain. The CED procedure involves a minimally invasive surgical exposure of the brain, followed by placement of small diameter catheters directly into the brain tumor. Subsequently, infusion of therapeutics into the tumor occurs over several hours to saturate the target tissue. As this approach effectively bypasses the blood-brain-barrier, it allows for delivery of macromolecular drugs that would not normally enter the brain to effectively reach high concentrations within brain tumor tissue. In order to reach similar concentrations as those achieved with CED, systemically administered conventional chemotherapeutic agents would need to be given at doses that would result in significant toxicity. Thus, CED simultaneously limits exposure of the remainder of the body to the therapeutic agent and thus minimizes systemic drug-related adverse effects.

A “patient” or “subject” herein refers to any organism to which a composition in accordance with the disclosure may be administered, e.g., for experimental, diagnostic, prophylactic, and/or therapeutic purposes. Similarly, “subject” or “patient” refers to an organism who may seek, who may require, who is receiving, or who will receive treatment or who is under care by a trained professional for a particular disease or condition. The patient or subject includes, any animals (for example, a mammal, such as a dog, a cat, a horse, a rabbit, a zoo animal, a cow, a pig, a sheep, a non-human primate, and a human), eligible for treatment who is experiencing or has experienced one or more signs, symptoms, or other indicators of a disease or disorder, such as a glioblastoma (GBM). In some embodiments, the subject is a human. In some embodiments, the subject, e.g., human, is experiencing or has experienced one or more signs, symptoms, or other indicators of a disease or disorder, such as a glioblastoma (GBM).

The terms “cancer” and “cancerous” refer to or describe the physiological condition in mammals that is typically characterized by unregulated cell growth. Included in this definition are benign and malignant cancers as well as dormant tumors or micrometastases. Examples of cancer include but are not limited to, carcinoma, lymphoma, blastoma, sarcoma, and leukemia. More particular examples of such cancers include, but are not limited to, glioblastoma (GBM), including, e.g., proneural GBM, neural GBM, classical GBM, and mesenchymal GBM. GBMs may be newly diagnosed, diagnosed, or recurrent. Other cancers include, for example, breast cancer, squamous cell cancer, lung cancer (including small-cell lung cancer, non-small cell lung cancer, adenocarcinoma of the lung, and squamous carcinoma of the lung), cancer of the peritoneum, hepatocellular cancer, gastric or stomach cancer (including gastrointestinal cancer), pancreatic cancer, ovarian cancer, cervical cancer, liver cancer, bladder cancer, hepatoma, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome.

“Tumor,” as used herein, refers to all neoplastic cell growth and proliferation, whether malignant or benign, and all pre-cancerous and cancerous cells and tissues. The terms “cancer,” “cancerous,” “cell proliferative disorder,” “proliferative disorder,” and “tumor” are not mutually exclusive as referred to herein.

The term “pharmaceutical formulation” refers to a sterile preparation that is in such form as to permit the biological activity of the medicament to be effective, and which contains no additional components that are unacceptably toxic to a subject to which the formulation would be administered.

The term “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically acceptable excipient,” refers to any ingredient other than active agents (e.g., as described herein) present in pharmaceutical compositions and having the properties of being substantially nontoxic and non-inflammatory in subjects. In some embodiments, pharmaceutically acceptable excipients are vehicles capable of suspending and/or dissolving active agents. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, and waters of hydration. Excipients include, but are not limited to: butylated hydroxytoluene (BHT), calcium carbonate, calcium phosphate (dibasic), calcium stearate, croscarmellose, cross-linked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and/or xylitol.

### II. COMPOSITIONS OF THE INVENTION

The present invention provides novel short cationic gamma-(γ)-modified peptide nucleic acids (sγPNA). The short cationic gamma-(γ)-modified peptide nucleic acids (sγPNA) specifically target the seed region of an oncomiR associated with glioblastoma (GBM) invasiveness and progression, e.g., miR21 and/or miR10b, with high affinity. The present invention also provides nanoparticles comprising the short cationic gamma-(γ)-modified peptide nucleic acids (sγPNA).

### Peptide Nucleic Acid

As used herein, peptide nucleic acids (PNAs) refer to a class of compounds that encompass nucleic acid analogues comprising ligands such as naturally occurring or synthetic DNA bases attached to a peptide backbone through a suitable linker (Nielsen P E, et al., Science 1991; 254: 1497-1500; U.S. Pat. No. 5,539,082). In some embodiments, PNAs are synthetic DNA analogs in which the phosphodiester backbone is replaced by repetitive units of N-(2-aminoethyl) glycine to which the purine and pyrimidine bases are attached via a methyl carbonyl linker.

In some embodiments, the peptide nucleic acids are gamma-(γ)-modified peptide nucleic acid, wherein a modification is introduced at the gamma-carbon of the backbone. Specifically, a simple modification within the gamma position of N-(2-aminoethyl) glycine backbone of PNA caused the single-stranded molecule to assume a pre-organized helical arrangement. Due to the stereogenic center, the gamma-PNA itself forms an alpha-helical structure, thereby reducing the self-aggregation, improving the solubility and forming a more stable duplex with the target DNA and RNA. These features provide higher binding affinity to the target. In addition, various modifications such as internal multi-labeling are possible at any gamma (γ)-position. In some embodiments, the PNA comprises a serine modification at the gamma position.

The synthetic backbone provides PNA with unique hybridization characteristics. Unlike DNA and RNA, the PNA backbone is not charged. Consequently, there is no electrostatic repulsion when PNAs hybridize to its target nucleic acid sequence, giving a higher stability to the PNA-DNA or PNA-RNA duplexes than the natural homo- or heteroduplexes. This greater stability is reflected by a higher thermal melting temperature (Tm), as compared to the corresponding DNA-DNA or DNA-RNA duplexes (Jensen K K, et al. Biochemistry 1997; 36: 5072-5077).

An additional consequence of the polyamide backbone is that PNAs hybridize virtually independently of the salt concentration. Thus, the Tm of PNA-DNA duplex is barely affected by low ionic strength. This property can be exploited when targeting DNA or RNA sequences involved in secondary structures, which are destabilized by low ionic strength. This facilitates the hybridization with the PNAs. The unnatural backbone of PNAs also means that PNAs are not degraded by nucleases or proteases. For instance, incubation of PNAs with Si nuclease or DNAse I has no effect on PNA (Demidov V, et al. Nucl Acids Res 1993; 21: 2103-2107). Owing to this resistance to the enzyme degradation, the lifetime of PNAs is extended both in vivo and in vitro. Also, PNAs are not recognized by polymerases and therefore cannot be directly used as primers or be copied.

PNAs hybridize to complementary DNA or RNA in a sequence-dependent manner, according to the Watson-Crick hydrogen bonding scheme. In contrast to DNA, PNA can bind in either parallel or antiparallel manner. However, the antiparallel binding is favored over the parallel one. Structural information on the PNA binding modes have been obtained by nuclear magnetic resonance and by X-ray crystallography. PNA are able to adopt both A- and B-type structures when associating with RNA and DNA, respectively, whereas PNA-PNA duplexes form an unusual helix conformation, called P-type and are characterized by a large pitch of 18 base pairs.

PNA probes can bind to either single-stranded DNA or RNA, or to double-stranded DNA. Homopyrimidine PNAs with a minimum of 10-mers, as well as PNAs containing a high proportion of pyrimidine residues, bind to complementary DNA sequences to form highly stable (PNA)2-DNA triplex helices displaying Tm over 70° C. In these triplexes, one PNA strand hybridizes to DNA through standard Watson-Crick base pairing rules, while the other PNA strand binds to DNA through Hoogsteen hydrogen bonds. The resulting structure is called P-loops. The stability of these triple helixes is so high that homopyrimidine PNA targeted to purine tracts of dsDNA invades the duplex by displacing one of the DNA strands. The efficiency of this strand invasion can be further enhanced by using two homopyrimidine PNA oligomers connected via a flexible linker or by the presence of nonstandard nucleobases in the PNA molecule.

In certain embodiments, the PNA comprises a sequence that is complementary to a region of the nucleic acid or nucleic acid segment. The nucleic acid can be, but is not limited to, single-stranded nucleic acid, double-stranded nucleic acid, polynucleotides, DNA, RNA, and single- or double-stranded viral nucleic acid. In certain embodiments, the nucleic acid can be a plasmid, a circular nucleic acid, a linear nucleic acid, a viral vector, a therapeutic vector, and the like.

In certain embodiments, the PNA comprises a sequence complementary to a region of a nucleic acid segment, e.g., a seed region of an oncomiR. In some embodiments, the oncomiR is selected from the group consisting of miR-21 and miR-10b.

In some embodiments, the nucleotide sequence targeting the seed region of the oncomiR comprises about 5, 6, 7, 8, 9, or 10 nucleotides in length. In one embodiment, the nucleotide sequence targeting the seed region of the oncomiR comprises about 5 nucleotides in length. In one embodiment, the nucleotide sequence targeting the seed region of the oncomiR comprises about 6 nucleotides in length. In one embodiment, the nucleotide sequence targeting the seed region of the oncomiR comprises about 7 nucleotides in length. In one embodiment, the nucleotide sequence targeting the seed region of the oncomiR comprises about 8 nucleotides in length. In one embodiment, the nucleotide sequence targeting the seed region of the oncomiR comprises about 9 nucleotides in length. In one embodiment, the nucleotide sequence targeting the seed region of the oncomiR comprises about 10 nucleotides in length.

In some embodiments, the PNA comprises a nucleotide sequence targeting the seed region of miR-21. The miR-21 sequence comprises the nucleic acid sequence of

| (SEQ ID NO: 1) | |
|---|---|
| | 5′-UAGCUUAUCAGACUGAUGUUGA-3′, |

where the seed region comprises the sequence of AGCUUAUC. In some embodiments, the nucleotide sequence targeting the seed region of miR-21 comprises GATAAGCT.

In some embodiments, the PNA comprises a nucleotide sequence targeting the seed region of miR-10b. The miR-10b sequence comprises the nucleic acid sequence of

| (SEQ ID NO: 2) | |
|---|---|
| | 5′-UACCCUGUAGAACCGAAUUUGUG-3′, |

where the seed region comprises the sequence of ACCCUGUA. In some embodiments, the nucleotide sequence targeting the seed region of miR-10b comprises TACAGGGT.

In some embodiments, the PNAs are cationic. The PNAs are further modified to increase its binding to the target nucleic acid, e.g., the seed region of the oncomiR, e.g., miR-21 or miR-10b, via electrostatic interaction between the cationic domain of PNAs and the negatively charged backbone in the flanking region of miRNA. In some embodiments, the PNAs are modified, e.g., extended with at least one, two or three positively charged amino acids, e.g., arginine residues, on the N-terminus. In some embodiments, the PNAs are modified, e.g., extended with at least one, two or three positively charged amino acids, e.g., arginine residues, on the C-terminus. In some embodiments, the PNAs comprises one arginine residue on the N-terminus and one arginine residue on the C-terminus. In some embodiments, the PNAs comprises two arginine residues on the N-terminus and one arginine residue on the C-terminus. In some embodiments, the PNAs comprises three arginine residues on the N-terminus and one arginine residue on the C-terminus.

In some embodiments, the peptide nucleic acid comprises from N-terminus (or 5′ end) to C-terminus (or 3′ end): at least one, two or three arginine residues, the nucleotide sequence targeting the seed region of the oncomiR, e.g., miR-21 or miR-10b, and at least one, two or three arginine residues at the C-terminus.

In some embodiments, the peptide nucleic acid comprises from N-terminus (or 5′ end) to C-terminus (or 3′ end): three arginine residues, the nucleotide sequence targeting the seed region of the oncomiR, e.g., miR-21 or miR-10b, and one arginine at the C-terminus.

Additional PNA sequences will be readily apparent to those of skill in the art for the implementation of the present invention with the nucleic acid of choice including, but not limited to, plasmids, therapeutic vectors, expression vectors, or the like. In some embodiments, the PNA comprises a nucleotide sequence of about 2 to about 40 nucleotides in length. In other embodiments, the PNA comprises a nucleotide sequence of about 3 to about 20 nucleotides in length. In certain embodiments, the PNA comprises a nucleotide sequence of about 4 to about 15 nucleotides in length. In some embodiments, the PNA comprises a nucleotide sequence of about 5 to about 10 nucleotides in length. In some embodiments, the PNA comprises a nucleotide sequence of about 5, about 6, about 7, about 8, about 9, or about 10 nucleotides in length.

Additional PNA types, modifications, and analogues useful as a PNA according to the system of the present invention are described in the following U.S. patent, the entire contents of each of which are incorporated herein by reference. U.S. Pat. No. 5,719,262 to Buchardt et al., describes PNAs having naturally-occurring nucleobases and non-naturally-occurring nucleobases attached to a polyamide backbone including amino acid (alkylamine) side chains. These PNAs are shown to have increased binding affinity to complementary DNA and RNA strands as well as increased sequence specificity and solubility. U.S. Pat. No. 5,766,855 to Buchardt et al., describes the synthesis of PNAs containing at least one 2,6-diaminopurine nucleobase and at least one C1-C8 alkylamine side chain which have enhanced binding affinity and sequence specificity. U.S. Pat. No. 5,714,331 to Buchardt et al., describes PNAs having enhanced binding affinity, sequence specificity and solubility. Methods of increasing specificity and binding affinity and solubility are provided. U.S. Pat. No. 5,705,333 to Buchardt et al., describes nucleic acid mimetics referred to as “PENAMS” which have a peptidic backbone and nucleotidic sidechains that are capable of hydrogen bonding to complementary nucleic acid sequences. The use of PENAMS in antisense and target nucleic acid isolation is suggested. U.S. Pat. No. 5,831,014 to Cook et al. and U.S. Pat. No. 5,539,083 to Cook et al. describe PNA combinatorial libraries and improved methods of synthesis of predefined PNA oligomers as well as random sequence PNA oligomers.

Methods for the synthesis of peptide nucleic acids are known to those with ordinary skill in the art. The procedures for PNA synthesis are similar to those employed for peptide synthesis, using standard solid-phase manual or automated synthesis, and is described in U.S. Pat. No. 5,539,082 to Nielsen, supra. The PNA molecules can routinely be labelled with biotin or fluorophores. Improvements in the method of PNA synthesis including PNA-peptide chimeras and improvements in nucleic acid binding specificity and affinity are described in the following U.S. Pat. Nos. 5,719,262; 5,766,855; 5,714,331; 5,705,333; 5,831,014; and 5,539,083, the entire contents of each of which are incorporated herein by reference.

Chemicals and instrumentation for the support bound automated chemical assembly of peptide nucleic acids are now commercially available. Both labeled and unlabeled PNA oligomers are likewise available from commercial vendors of custom PNA oligomers. Chemical assembly of a PNA is analogous to solid phase peptide synthesis, wherein at each cycle of assembly the oligomer possesses a reactive alkyl amino terminus that is condensed with the next synthon to be added to the growing polymer.

PNA may be synthesized at any scale, from submicromole to millimole, or more. PNA can be conveniently synthesized at the 2 mole scale, using Fmoc(Boc) protecting group monomers on an Expedite Synthesizer (Applied Biosystems) using a XAL, PAL or many other suitable commercially available peptide synthesis supports. Alternatively, the Model 433A Synthesizer (Applied Biosystems) with a suitable solid support (e.g. MBHA support) can be used. Moreover, many other automated synthesizers and synthesis supports can be utilized. Synthesis can be performed using continuous flow method and/or a batch method. PNA can also be manually synthesized.

In some embodiments, the PNAs are labeled with a detectable moiety. In some embodiments, the PNAs are not labeled. Non-limiting methods for labeling PNAs are described in U.S. Pat. Nos. 6,110,676, 6,361,942, 6,355,421, 6,969,766, and 7,022,851 the examples section of this specification or are otherwise well known in the art of PNA synthesis and peptide synthesis.

Non-limiting examples of detectable moieties (labels) suitable for labeling PNA used in the practice of this invention would include a dextran conjugate, a branched nucleic acid detection system, a chromophore, a fluorophore, a spin label, a radioisotope, an enzyme, a hapten, an acridinium ester and a chemiluminescent compound. Other suitable labeling reagents and preferred methods of attachment would be recognized by those of ordinary skill in the art of PNA, peptide or nucleic acid synthesis. Examples of haptens include 5 (6)-carboxyfluorescein, 2,4-dinitrophenyl, digoxigenin, and biotin. Exemplary fluorochromes (fluorophores) include 5 (6)-carboxyfluorescein (Flu), 6-((7-amino-4-methylcoumarin-3-acetyl)amino) hexanoic acid (Cou), 5 (and 6)-carboxy-X-rhodamine (Rox), Cyanine 2 (Cy2) Dye, Cyanine 3 (Cy3) Dye, Cyanine 3.5 (Cy3.5) Dye, Cyanine 5 (Cy5) Dye, Cyanine 5.5 (Cy5.5) Dye Cyanine 7 (Cy7) Dye, Cyanine 9 (Cy9) Dye (Cyanine dyes 2,3,3,5,5 and 5.5 are available as NHS esters from Amersham, Arlington Heights, Ill.), JOE, Tamara or the Alexa dye series (Molecular Probes, Eugene, Oreg.). Exemplary enzymes include polymerases (e.g. Taq polymerase, Klenow PNA polymerase, T7 DNA polymerase, Sequenase, DNA polymerase 1 and phi29 polymerase), alkaline phosphatase (AP), horseradish peroxidase (HRP) and most preferably, soy bean peroxidase (SBP).

### Nanoparticles

The present invention also provides nanoparticles comprising a short gamma-(γ)-modified peptide nucleic acid (sγPNA) which specifically targets the seed region of an oncomiR associated with glioblastoma (GBM) invasiveness and progression, e.g., miR21 and/or miR10b. The inventors of the present invention have also surprisingly discovered that, by packaging these novel sγPNAs into a nanoparticle, for example, a bioadhesive nanoparticle (BNP), the resulting sγPNAs loaded BNPs produced superior anti-miR efficacy in tumor cells and remarkable cytotoxicity when combined with a chemotherapeutic agent, as exemplified by temozolomide (TMZ). Indeed, as set forth in the Examples herein, when delivered in vivo, the sγPNA BNPs dramatically increased the survival in mice with intracranial GBM. Moreover, the combination of sγPNA BNPs with a chemotherapeutic agent such as temozolomide (TMZ) suppressed tumor growth and significantly improved the survival of GBM mice beyond 120 days with substantial improvement in histopathology. Thus, the inventors of the present invention provides a promising approach to improve the treatment of GBM. with a potential to personalize treatment based on tumor specific oncomiRs.

The nanoparticles of the present invention can be made from any polymers known in the art. In some embodiments, the nanoparticles may comprise one or more biocompatible and/or biodegradable synthetic polymers, including, for example, polycarbonates (e.g., poly(1,3-dioxan-2one)), polyanhydrides (e.g., poly(sebacic anhydride)), polyhydroxyacids (e.g., poly((β-hydroxyalkanoate)), polypropylfumarates, polycaprolactones, polyamides (e.g., polycaprolactam), polyacetals, polyethers, polyesters (e.g., polylactide and polyglycolide), biodegradable polycyanoacrylates, polyvinyl alcohols, and biodegradable polyurethanes. For example, the nanoparticles may comprise one or more of the following biodegradable polymers: poly(lactic acid), poly(glycolic acid), poly(caprolactone), poly(lactide-co-glycolide), poly(lactide-co-caprolactone), poly(glycolide-co-caprolactone), and poly(D,L-lactide-co-glycolide).

In some embodiments, the nanoparticles may comprise one or more acrylic polymers. In certain embodiments, acrylic polymers include, for example, acrylic acid and methacrylic acid copolymers, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamide copolymer, poly(methyl methacrylate), poly(methacrylic acid anhydride), methyl methacrylate, polymethacrylate, poly(methyl methacrylate) copolymer, polyacrylamide, aminoalkyl methacrylate copolymer, glycidyl methacrylate copolymers, and combinations comprising one or more of the foregoing polymers. The acrylic polymer may comprise fully-polymerized copolymers of acrylic and methacrylic acid esters with a low content of quaternary ammonium groups.

In some embodiments, the nanoparticles may comprise a polyester. Exemplary such polyesters include, for example, polyalkylene glycols, poly(glycolide-co-lactide), poly(lactic-co-glycolic acid)-PEG copolymers, poly(lactic acid), poly(lactic acid)-PEG copolymers, poly(glycolic acid), poly(glycolic acid)-PEG copolymers, co-polymers of polylactic and polyglycolic acid, and derivatives thereof. In some embodiments, polyesters include, for example, polyanhydrides, poly(ortho ester), poly(ortho ester)-PEG copolymers, poly(caprolactone), poly(caprolactone)-PEG copolymers, polylysine, polylysine-PEG copolymers, poly(ethylene imine), poly(ethylene imine)-PEG copolymers, and derivatives thereof. In some embodiments, polyesters may include, for example, polycaprolactone, poly(L-lactide-co-L-lysine), poly(serine ester), poly(4-hydroxy-L-proline ester), poly[α-(4-aminobutyl)-L-glycolic acid], and derivatives thereof.

The nanoparticles described herein are biodegradable and/or biocompatible, i.e., a nanoparticle containing polymers that do not typically induce an adverse response when inserted or injected into a living subject, for example, without significant inflammation and/or acute rejection of the polymers by the immune system, for instance, via a T-cell response. One simple test to determine biocompatibility is to expose polymers to cells in vitro, where biocompatible polymers typically do not result in significant cell death at moderate concentrations. For example, a biocompatible polymer may cause less than about 20% cell death when exposed to cells such as fibroblasts or epithelial cells, even if phagocytosed or otherwise taken-up by such cells.

The polymers present in the nanoparticles can also be biodegradable, i.e., the polymers are able to degrade, chemically and/or biologically, within a physiological environment, such as within the body. For instance, the polymers can hydrolyze spontaneously upon exposure to water (e.g., within a subject) and/or the polymers can degrade upon exposure to heat (e.g., at temperatures of about 37° C.). Degradation of the polymers can occur at varying rates, depending on the polymers or copolymers used. For example, the half-life of the polymers (the time at which 50% of the polymers are degraded into monomers and/or other nonpolymeric moieties) can be on the order of days, weeks, months, or years, depending on the particular polymers used to make the nanoparticles. The polymers can be biologically degraded, e.g., by enzymatic activity or cellular machinery, in some cases, for example, through exposure to a lysozyme (e.g., having relatively low pH). In some cases, the polymers can be broken down into monomers and/or other nonpolymeric moieties that cells can either reuse or dispose of without significant toxic effect on the cells (e.g., polylactide can be hydrolyzed to form lactic acid, and polyglycolide can be hydrolyzed to form glycolic acid).

In some embodiments, the nanoparticles comprise a poly-lactic acid and hyperbranched polyglycerol (PLA-HPG) polymer. In some embodiments, the nanoparticles are modified or functionalized with aldehyde groups, creating PLA-HPG-CHO. Nanoparticles formed from PLA-HPG-CHO are bioadhesive. In some embodiments, bioadhesive nanoparticles (BNP) are produced by oxidation of the nonadhesive nanoparticles, e.g., nanoparticles made of PLA-HPG polymers, which resulting in conversion of vicinal diols on the surface of the nonadhesive nanoparticles into aldehydes on the BNPs, e.g., creating PLA-HPG-CHO. Aldehydes spontaneously react with proteins to form a variety of bonds, such as Schiff-base bonds; therefore, the presence of surface aldehydes on BNPs leads to particle adhesion to protein-rich materials. In some embodiments, NPs formed from PLA-HPG-CHO are bioadhesive. In some embodiments, successful conversion to the bioadhesive state can be confirmed by measuring NP adhesion to poly(L-lysine) coated glass (see Example 1).

The nanoparticles may have any zeta potential. The nanoparticles can have a zeta potential from −300 mV to +300 mV, −100 mV to +100 mV, from −50 mV to +50 mV, from −40 mV to +40 mV, from −30 mV to +30 mV, from −20 mV to +20 mV, from −10 mV to +10 mV, or from −5 mV to +5 mV. The nanoparticles can have a negative or positive zeta potential. In some embodiments the nanoparticles have a substantially neutral zeta potential, i.e. the zeta potential is approximately 0 mV. In preferred embodiments the nanoparticles have a zeta potential of approximately −30 to about 30 mV, preferably from about −20 to about 20 mV, more preferably from about −10 to about 10 mV.

The nanoparticles may have any diameter. The nanoparticles can have an average diameter of between about 1 nm and about 1000 microns, about 1 nm and about 100 microns, about 1 nm and about 10 microns, about 1 nm and about 1000 nm, about 1 nm and about 500 nm, about 1 nm and about 250 nm, or about 1 nm and about 100 nm. In certain embodiments, the particle is a nanoparticle having a diameter from about 25 nm to about 250 nm, about 20 nm to about 200 nm, about 40 nm to about 200 nm, about 20 nm to about 100 nm, about 30 nm to about 90 nm, about 40 nm to about 80 nm, about 50 nm to about 70 nm, or about 40 nm to about 60 nm.

For administration to the brain, particularly when delivered locally by injection, infusion, or convection enhanced delivery, the nanoparticles have a diameter from about 25 nm to about 120 nm, from about 40 nm to about 100 nm, or from about 60 nm to about 90 nm, or from about 35 nm to about 60 nm. In some applications, particularly those in non-tumor regions of the brain, the particles are larger than those for administration into the tumor.

In some embodiments, particles size typically is based on a population, wherein 60, 70, 80, 85, 90, or 95% of the population has the desired size range.

The polydispersity can be from about 0.01 to 0.30, or from about 0.01 to about 0.25, or from about 0.01 to about 0.20, or from about 0.01 to about 0.15, or from about 0.01 to about 0.10.

Methods of making polymeric particles are known in the art. Common microencapsulation techniques include, but are not limited to, spray drying, interfacial polymerization, hot melt encapsulation, phase separation encapsulation (spontaneous emulsion microencapsulation, solvent evaporation microencapsulation, and solvent removal microencapsulation), nano-precipitation, coacervation, low temperature microsphere formation, and phase inversion nanoencapsulation (PIN). Other emulsion emulsion-based procedures are described below.

### 1. Phase Separation Microencapsulation

In phase separation microencapsulation techniques, a polymer solution is stirred, optionally in the presence of one or more active agents to be encapsulated. While continuing to uniformly suspend the material through stirring, a nonsolvent for the polymer is slowly added to the solution to decrease the polymer's solubility. Depending on the solubility of the polymer in the solvent and nonsolvent, the polymer either precipitates or phase separates into a polymer rich and a polymer poor phase. Under proper conditions, the polymer in the polymer rich phase will migrate to the interface with the continuous phase, encapsulating the active agent(s) in a droplet with an outer polymer shell.

### 2. Spontaneous Emulsion Microencapsulation

Spontaneous emulsification involves solidifying emulsified liquid polymer droplets formed above by changing temperature, evaporating solvent, or adding chemical cross-linking agents. The physical and chemical properties of the encapsulant, as well as the properties of the one or more active agents optionally incorporated into the nascent particles, dictates suitable methods of encapsulation. Factors such as hydrophobicity, molecular weight, chemical stability, and thermal stability affect encapsulation.

### 3. Solvent Evaporation Microencapsulation

Methods for forming microspheres using solvent evaporation techniques are described in E. Mathiowitz et al., J. Scanning Microscopy, 4:329 (1990); L. R. Beck et al., Fertil. Steril., 31:545 (1979); L. R. Beck et al, Am. J Obstet. Gynecol., 135(3) (1979); S. Benita et al., J. Pharm. Sci., 73:1721 (1984); and U.S. Pat. No. 3,960,757 to Morishita et al. The polymer is dissolved in a volatile organic solvent, such as methylene chloride. One or more active agents to be incorporated are optionally added to the solution, and the mixture is suspended in an aqueous solution that contains a surface active agent such as poly(vinyl alcohol). The resulting emulsion is stirred until most of the organic solvent evaporated, leaving solid microparticles/nanoparticles. This method is useful for relatively stable polymers like polyesters and polystyrene.

### 4. Phase Inversion Nanoencapsulation (PIN)

Nanoparticles can also be formed using the phase inversion nanoencapsulation (PIN) method, wherein a polymer is dissolved in a “good” solvent, fine particles of a substance to be incorporated, such as a drug, are mixed or dissolved in the polymer solution, and the mixture is poured into a strong non solvent for the polymer, to spontaneously produce, under favorable conditions, polymeric microspheres, wherein the polymer is either coated with the particles or the particles are dispersed in the polymer. See, e.g., U.S. Pat. No. 6,143,211 to Mathiowitz, et al. The method can be used to produce monodisperse populations of nanoparticles and microparticles in a wide range of sizes, including, for example, about 100 nanometers to about 10 microns.

### 5. Microfluidics

Nanoparticles can be prepared using microfluidic devices. A polymeric material is mixed with a drug or drug combinations in a water miscible organic solvent. The water miscible organic solvent can be one or more of the following: acetone, ethanol, methanol, isopropyl alcohol, acetonitrile and Dimethyl sulfoxide (DMSO). The resulting mixture solution is then added to an aqueous solution to yield nanoparticle solution. The targeted peptides or fluorophores or drugs may be associated with the surface of, encapsulated within, surrounded by, and/or distributed throughout the polymeric matrix of the particles.

### 6. Nanoprecipitation

In nanoprecipitation, the polymer and active agent (e.g., nucleic acids) are co-dissolved in a selected, water-miscible solvent, for example DMSO, acetone, ethanol, acetone, etc. In a preferred embodiment, active agent and polymer are dissolved in DMSO. The solvent containing the polymer and active agent is then drop-wise added to an excess volume of stirring aqueous phase containing a stabilizer (e.g., poloxamer, Pluronic®, and other stabilizers known in the art). Particles are formed and precipitated during solvent evaporation. To reduce the loss of polymer, the viscosity of the aqueous phase can be increased by using a higher concentration of the stabilizer or other thickening agents such as glycerol and others known in the art. Lastly, the entire dispersed system is centrifuged, and the nucleic acid-loaded polymer nanoparticles are collected and optionally filtered. Nanoprecipitation-based techniques are discussed in, for example, U.S. Pat. No. 5,118,528.

### III. PHARMACEUTICAL COMPOSITIONS OF THE INVENTION

The present invention also includes pharmaceutical compositions and formulations which include the peptide nucleic acids (PNAs) and/or the nanoparticles comprising the PNAs of the invention.

In one embodiment, provided herein are pharmaceutical compositions containing a peptide nucleic acid, e.g., a short cationic gamma-(γ)-modified peptide nucleic acids (sγPNA) which specifically target the seed region of an oncomiR, e.g., miR21 and/or miR10b, and a pharmaceutically acceptable carrier.

In another embodiment, provided herein are pharmaceutical compositions containing a nanoparticle comprising a peptide nucleic acid, e.g., a short cationic gamma-(γ)-modified peptide nucleic acids (sγPNA) which specifically target the seed region of an oncomiR, e.g., miR21 and/or miR10b, and a pharmaceutically acceptable carrier.

As would be appreciated by one of skill in this art, the pharmaceutically acceptable carrier or excipient can be chosen based on the particular route of administration, the location of the target issue, the composition being delivered, and the desired time course of delivery of the composition.

The phrase “pharmaceutically acceptable” is employed herein to refer to those compounds, materials, compositions, and/or dosage forms which are, within the scope of sound medical judgment, suitable for use in contact with the tissues of human subjects and animal subjects without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the subject being treated. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum components, such as serum albumin, HDL and LDL; and (22) other non-toxic compatible substances employed in pharmaceutical formulations.

The pharmaceutical compositions can also contain a wetting agent, an emulsifier, and a lubricant, such as sodium lauryl sulfate and magnesium stearate, as well as coloring agents, release agents, coating agents, sweetening, flavoring, and perfuming agents, preservatives, and antioxidants. Non-limiting examples of pharmaceutically-acceptable antioxidants that can be included in any of the compositions described herein include: water soluble antioxidants, such as ascorbic acid, cysteine hydrochloride, sodium bisulfate, sodium metabisulfite, sodium sulfite, and the like; oil-soluble antioxidants, such as ascorbyl palmitate, butylated hydroxyanisole (BHA), butylated hydroxytoluene (BHT), lecithin, propyl gallate, alpha-tocopherol (e.g., alpha-tocopheryl succinate), and the like; and metal chelating agents, such as citric acid, ethylenediamine tetraacetic acid (EDTA), sorbitol, tartaric acid, phosphoric acid, and the like.

The pharmaceutical compositions containing the peptide nucleic acids (PNAs) and/or the nanoparticles comprising the PNAs are useful for treating a disease, e.g., cancer, e.g., glioblastoma. Such pharmaceutical compositions are formulated based on the mode of delivery.

The pharmaceutical compositions of the present invention may be administered by any suitable means, including but not limited to, intravenous injection, intramuscular injection, subcutaneously injection, intraperitoneal injection, or infusion. In some embodiments, pharmaceutical compositions of the present invention may be administered by intra-arterial injection, intraventricular injection, intracerebral injection, or intrathecal injection. In some embodiments, pharmaceutical compositions of the present invention may be administered orally, interdermally, rectally, vaginally, mucosally, nasally, buccally, enterally, or sublingually. In some embodiments, nanoparticle compositions of the present invention may be administered by intratracheal instillation, bronchial instillation, and/or inhalation; and/or as an oral spray, nasal spray, and/or aerosol.

In some embodiments, the pharmaceutical compositions are administered by convection-enhanced drug delivery (CED) to the brain. As used herein, the term “convection enhanced delivery” or “CED’ refers to a therapeutic strategy that was developed to facilitate targeted delivery of pharmaceuticals to the brain. The CED procedure involves a minimally invasive surgical exposure of the brain, followed by placement of small diameter catheters directly into the brain tumor. Subsequently, infusion of therapeutics into the tumor occurs over several hours to saturate the target tissue. As this approach effectively bypasses the blood-brain-barrier, it allows for delivery of macromolecular drugs that would not normally enter the brain to effectively reach high concentrations within brain tumor tissue. In order to reach similar concentrations as those achieved with CED, systemically administered conventional chemotherapeutic agents would need to be given at doses that would result in significant toxicity. Thus, an additional benefit of CED is that it simultaneously limits exposure of the remainder of the body to the therapeutic agent and thus minimizes systemic drug-related adverse effects.

Nanoparticles for CED delivery are typically less than about 100 nm in diameter, for example in a range of about 60 to about 90 nm diameter. Additionally or alternatively, additives such as trehalose, other sugars, and other aggregation-reducing materials can be added to any solution including particles, for example, a resuspension solution and/or a pharmaceutical composition for administration to subject in need thereof to enhance CED of the particles.

The pharmaceutical compositions of the present invention can be formulated in dosages, generally, at the maximum amount while avoiding or minimizing any potentially detrimental side effects. The compositions can be administered in effective amounts, alone or in a cocktail with one or more additional anti-cancer agents (e.g., any of the exemplary anti-cancer agents described herein or known in the art). An effective amount is generally an amount sufficient to decrease cancer cell proliferation (e.g., in a human subject), induce cancer cell death (e.g., in a human subject), or treat cancer in a subject (e.g., a human).

One of skill in the art can determine what an effective dosage of the pharmaceutical composition is by screening the composition using any of the assays described herein or other known assays. The effective amount may depend, of course, on factors such as the stage of the cancer being treated, individual patient parameters including age, physical condition, sex, size, and mass, concurrent anti-cancer treatments, the rate of excretion or metabolism of the composition being employed, the duration of the treatment, the time of administration, the frequency of treatment, the activity of the particular composition, the mode of administration, prior medical history of the subject being treated, and like factors well known in the medical arts. These factors are well known to those of ordinary skill in the art and can be addressed with no more than routine experimentation. In some cases, a maximum dosage be used, that is, the highest safe dosage according to sound medical judgment.

### IV. METHODS OF THE INVENTION

The present invention provides a method of treating a disease in a subject in need thereof, comprising administering to the subject a therapeutically effective amount of the nanoparticle, or the pharmaceutical composition of the present invention, thereby treating the disease in the subject in need thereof.

In some embodiments, the disease is cancer. Examples of cancers that may be treated using the compositions and methods disclosed herein include, but are not limited to glioblastoma (GBM), including, e.g., proneural GBM, neural GBM, classical GBM, and mesenchymal GBM, squamous cell cancer, small-cell lung cancer, pituitary cancer, esophageal cancer, astrocytoma, soft tissue sarcoma, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, renal cancer, liver cancer, prostate cancer, vulval cancer, thyroid cancer, hepatic carcinoma, brain cancer, endometrial cancer, testis cancer, cholangiocarcinoma, gallbladder carcinoma, gastric cancer, melanoma, and various types of head and neck cancer. In some embodiments, lung cancer is non-small cell lung cancer or lung squamous cell carcinoma. In some embodiments, leukemia is acute myeloid leukemia or chronic lymphocytic leukemia. In some embodiments, breast cancer is breast invasive carcinoma. In some embodiments, ovarian cancer is ovarian serous cystadenocarcinoma. In some embodiments, kidney cancer is kidney renal clear cell carcinoma. In some embodiments, colon cancer is colon adenocarcinoma. In some embodiments, bladder cancer is bladder urothelial carcinoma. In some embodiments, the cancer is selected from bladder cancer, cervical cancer (such as squamous cell cervical cancer), head and neck squamous cell carcinoma, rectal adenocarcinoma, non-small cell lung cancer, endometrial cancer, prostate adenocarcinoma, colon cancer, ovarian cancer (such as serous epithelial ovarian cancer), and melanoma. In some embodiments, the cancer is glioblastoma.

In another aspect, the present invention provides a method of reducing a tumor growth in a subject in need thereof, comprising administering to the subject a therapeutically effective amount of the nanoparticle, or the pharmaceutical composition of the present invention, thereby reducing the tumor growth in the subject in need thereof.

In some embodiments, administration of the nanoparticle, or the pharmaceutical composition of the present invention results in at least 5%, 10%, 15%, 20%, 25%, 30%, 35%, 40%, 45%, 50%, 55%, 60%, 65%, 70%, 75%, 80%, 85%, 90%, or 95% reduction in tumor volume or size, or tumor growth.

In one aspect, the present invention provides a method of prolonging survival time of a subject in need thereof, comprising administering to the subject a therapeutically effective amount of the nanoparticle, or the pharmaceutical composition of the present invention, thereby prolonging survival time of the subject in need thereof.

In some embodiments, administration of the nanoparticle, or the pharmaceutical composition of the present invention results in at least 5%, 10%, 15%, 20%, 25%, 30%, 35%, 40%, 45%, 50%, 55%, 60%, 65%, 70%, 75%, 80%, 85%, 90%, or 95% increase in survival time of the subject.

In another aspect, the present invention provides a method of increasing sensitivity to a chemotherapy in a subject in need thereof, comprising administering to the subject a therapeutically effective amount of the nanoparticle, or the pharmaceutical composition of the present invention, thereby increasing sensitivity to the chemotherapy in the subject in need thereof.

In some embodiments, administration of the nanoparticle, or the pharmaceutical composition of the present invention results in at least 5%, 10%, 15%, 20%, 25%, 30%, 35%, 40%, 45%, 50%, 55%, 60%, 65%, 70%, 75%, 80%, 85%, 90%, or 95% increase in sensitivity to a chemotherapy, e.g., temozolomide.

In one aspect, the present invention provides a method of increasing apoptosis of tumor cells in a subject in need thereof, comprising administering to the subject a therapeutically effective amount of the nanoparticle, or the pharmaceutical composition of the present invention, thereby increasing apoptosis of the tumor cells in the subject in need thereof.

In some embodiments, administration of the nanoparticle, or the pharmaceutical composition of the present invention results in at least 5%, 10%, 15%, 20%, 25%, 30%, 35%, 40%, 45%, 50%, 55%, 60%, 65%, 70%, 75%, 80%, 85%, 90%, or 95% increase in apoptosis in tumor cells.

In some embodiments, administration of the nanoparticle, or the pharmaceutical composition of the present invention results in at least 5%, 10%, 15%, 20%, 25%, 30%, 35%, 40%, 45%, 50%, 55%, 60%, 65%, 70%, 75%, 80%, 85%, 90%, or 95% reduction in the expression level of miR-10b and/or miR21.

In some embodiments, administration of the nanoparticle, or the pharmaceutical composition of the present invention results in at least 5%, 10%, 15%, 20%, 25%, 30%, 35%, 40%, 45%, 50%, 55%, 60%, 65%, 70%, 75%, 80%, 85%, 90%, or 95% reduction in the expression level of VEGFA and/or ITGB8.

In some embodiments, the subject is a human subject.

In some embodiments, the methods further comprising administering to the subject an additional therapeutic agent or treatment. Examples of additional therapeutic agents, e.g., anti-cancer therapies, include, without limitation, surgery, radiation therapy (radiotherapy), biotherapy, immunotherapy, or one or more additional anti-cancer agents, such as a chemotherapeutic agent, a growth inhibitory agent, an anti-angiogenesis agent and/or an anti-neoplastic composition. Nonlimiting examples of anti-cancer agents, chemotherapeutic agents, growth inhibitory agents, anti-angiogenesis agents, and anti-neoplastic compositions that can be used in combination with the nanoparticles and pharmaceutical compositions of the present invention are as follows.

A “chemotherapeutic agent” is a chemical compound useful in the treatment of cancer. Examples of chemotherapeutic agents include, but are not limited to, temozolomide (TMZ), alkylating agents such as thiotepa and Cytoxan® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Agnew, Chem Intl. Ed. Engl., 33: 183-186 (1994)); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, Adriamycin® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, OR); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., Taxol® paclitaxel (Bristol-Myers Squibb Oncology, Princeton, N.J.), Abraxane® Cremophor-free, albumin-engineered nanoparticle formulation of paclitaxel (American Pharmaceutical Partners, Schaumberg, Illinois), and Taxotere® doxetaxel (Rhône-Poulenc Rorer, Antony, France); chloranbucil; Gemzar® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; Navelbine® vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluorometlhylornithine (DMFO); retinoids such as retinoic acid; capecitabine; combretastatin; leucovorin (LV); oxaliplatin, including the oxaliplatin treatment regimen (FOLFOX); inhibitors of PKC-alpha, Raf, H-Ras, EGFR (e.g., erlotinib (Tarceva®)) and VEGF-A that reduce cell proliferation and pharmaceutically acceptable salts, acids or derivatives of any of the above.

Further nonlimiting exemplary chemotherapeutic agents include anti-hormonal agents that act to regulate or inhibit hormone action on cancers such as anti-estrogens and selective estrogen receptor modulators (SERMs), including, for example, tamoxifen (including Nolvadex® tamoxifen), raloxifene, droloxifene, 4-hydroxytamoxifen, trioxifene, keoxifene, LY117018, onapristone, and Fareston® toremifene; aromatase inhibitors that inhibit the enzyme aromatase, which regulates estrogen production in the adrenal glands, such as, for example, 4(5)-imidazoles, aminoglutethimide, Megase® megestrol acetate, Aromasin® exemestane, formestanie, fadrozole, Rivisor® vorozole, Femara® letrozole, and Arimidex® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Ralf and H-Ras; ribozymes such as a VEGF expression inhibitor (e.g., Angiozyme® ribozyme) and a HER2 expression inhibitor; vaccines such as gene therapy vaccines, for example, Allovectin® vaccine, Leuvectin® vaccine, and Vaxid® vaccine; Proleukin® rIL-2; Lurtotecan® topoisomerase 1 inhibitor; Abarelix® rmRH; and pharmaceutically acceptable salts, acids or derivatives of any of the above.

In some embodiments, the nanoparticles and pharmaceutical compositions of the invention may be further administered with gemcitabine-based chemotherapy in which one or more chemotherapy agents including gemcitabine or including gemcitabine and nab-paclitaxel are administered. In some such embodiments, the nanoparticles and pharmaceutical compositions of the invention may be administered with at least one chemotherapy agent selected from gemcitabine, nab-paclitaxel, leukovorin (folinic acid), 5-fluorouracil (5-FU), irinotecan, and oxaliplatin. FOLFIRINOX is a chemotherapy regime comprising leukovorin, 5-FU, irinotecan (such as liposomal irinotecan injection), and oxaliplatin. In some embodiments, the nanoparticles and pharmaceutical compositions of the invention may be further administered with gemcitabine-based chemotherapy. In some embodiments, the nanoparticles and pharmaceutical compositions of the invention may be further administered with at least one agent selected from (a) gemcitabine; (b) gemcitabine and nab-paclitaxel; and (c) FOLFIRINOX. In some embodiments, the at least one agent is gemcitabine. In some such embodiments, the cancer to be treated is pancreatic cancer.

An “anti-angiogenesis agent” or “angiogenesis inhibitor” refers to a small molecular weight substance, a polynucleotide (including, e.g., an inhibitory RNA (RNAi or siRNA)), a polypeptide, an isolated protein, a recombinant protein, an antibody, or conjugates or fusion proteins thereof, that inhibits angiogenesis, vasculogenesis, or undesirable vascular permeability, either directly or indirectly. It should be understood that the anti-angiogenesis agent includes those agents that bind and block the angiogenic activity of the angiogenic factor or its receptor. For example, an anti-angiogenesis agent is an antibody or other antagonist to an angiogenic agent, e.g., antibodies to VEGF-A (e.g., bevacizumab (Avastin®)) or to the VEGF-A receptor (e.g., KDR receptor or Flt-1 receptor), anti-PDGFR inhibitors such as Gleevec® (Imatinib Mesylate), small molecules that block VEGF receptor signaling (e.g., PTK787/ZK2284, SU6668, Sutent®/SU11248 (sunitinib malate), AMG706, or those described in, e.g., international patent application WO 2004/113304). Anti-angiogensis agents also include native angiogenesis inhibitors, e.g., angiostatin, endostatin, etc. See, e.g., Klagsbrun and D'Amore (1991) Annu. Rev. Physiol. 53:217-39; Streit and Detmar (2003) Oncogene 22:3172-3179 (e.g., Table 3 listing anti-angiogenic therapy in malignant melanoma); Ferrara & Alitalo (1999) Nature Medicine 5(12):1359-1364; Tonini et al. (2003) Oncogene 22:6549-6556 (e.g., Table 2 listing known anti-angiogenic factors); and, Sato (2003) Int. J. Clin. Oncol. 8:200-206 (e.g., Table 1 listing anti-angiogenic agents used in clinical trials).

A “growth inhibitory agent” as used herein refers to a compound or composition that inhibits growth of a cell (such as a cell expressing VEGF) either in vitro or in vivo. Thus, the growth inhibitory agent may be one that significantly reduces the percentage of cells (such as a cell expressing VEGF) in S phase. Examples of growth inhibitory agents include, but are not limited to, agents that block cell cycle progression (at a place other than S phase), such as agents that induce G1 arrest and M-phase arrest. Classical M-phase blockers include the vincas (vincristine and vinblastine), taxanes, and topoisomerase II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in Mendelsohn and Israel, eds., The Molecular Basis of Cancer, Chapter 1, entitled “Cell cycle regulation, oncogenes, and antineoplastic drugs” by Murakami et al. (W.B. Saunders, Philadelphia, 1995), e.g., p. 13. The taxanes (paclitaxel and docetaxel) are anticancer drugs both derived from the yew tree. Docetaxel (Taxotere®, Rhone-Poulenc Rorer), derived from the European yew, is a semisynthetic analogue of paclitaxel (Taxol®, Bristol-Myers Squibb). Paclitaxel and docetaxel promote the assembly of microtubules from tubulin dimers and stabilize microtubules by preventing depolymerization, which results in the inhibition of mitosis in cells.

The term “anti-neoplastic composition” refers to a composition useful in treating cancer comprising at least one active therapeutic agent. Examples of therapeutic agents include, but are not limited to, e.g., chemotherapeutic agents, growth inhibitory agents, cytotoxic agents, agents used in radiation therapy, anti-angiogenesis agents, cancer immunotherapeutic agents, apoptotic agents, anti-tubulin agents, and other-agents to treat cancer, such as anti-HER-2 antibodies, anti-CD20 antibodies, an epidermal growth factor receptor (EGFR) antagonist (e.g., a tyrosine kinase inhibitor), HER1/EGFR inhibitor (e.g., erlotinib (Tarceva®), platelet derived growth factor inhibitors (e.g., Gleevec® (Imatinib Mesylate)), a COX-2 inhibitor (e.g., celecoxib), interferons, cytokines, antagonists (e.g., neutralizing antibodies) that bind to one or more of the following targets ErbB2, ErbB3, ErbB4, PDGFR-beta, BlyS, APRIL, BCMA, or VEGF receptor(s), and other bioactive and organic chemical agents, etc. Combinations thereof are also included in the invention.

The combined administration includes co-administration, using separate formulations or a single pharmaceutical formulation, and consecutive administration in either order.

In one embodiment, the methods comprise administering the nanoparticle or pharmaceutical compositions of the present invention in combination with one or more chemotherapeutic agents (e.g., TMZ). The combination therapy may provide “synergy” and prove “synergistic,” i.e., the effect achieved when the active ingredients used together is greater than the sum of the effects that results from using the compounds separately. A synergistic effect may be attained when the active ingredients are: (1) co-formulated and administered or delivered simultaneously in a combined, unit dosage formulation; (2) delivered by alternation or in parallel as separate formulations; or (3) by some other regimen. When delivered in alternation therapy, a synergistic effect may be attained when the compounds are administered or delivered sequentially, e.g., by different injections in separate syringes. In general, during alternation therapy, an effective dosage of each active ingredient is administered sequentially, i.e., serially, whereas in combination therapy, effective dosages of two or more active ingredients are administered together.

The methods of treatment of the invention result in, for example, a reduction in tumor size (e.g., glioblastoma tumor size), prolonged survival time, and/or increased sensitivity to chemotherapy in the treated patient. Furthermore, the combination of the nanoparticle or pharmaceutical compositions of the present invention and the chemotherapy, e.g., TMZ, result in a remarkable delay of tumor growth and prevention of disease relapse, thus providing an additive or synergistic therapeutic benefit to the patient.

The present disclosure is further illustrated by the following non-limiting examples.

### EXAMPLES

### Example 1: Anti-Seed PNAs Targeting Multiple oncomiRs for Brain Tumor Therapy

MicroRNAs (miRNAs or miRs) are ˜20-25 nucleotide non-coding RNAs that regulate gene expression at the post-transcriptional level (5). The dysregulation of miRs, either upregulation (where they are known as oncomiRs) (6) or downregulation, plays an important role in several malignancies (7-10). Previous studies have reported aberrant miR expression levels in GBM patients; some of these are associated with poor prognosis and low overall survival (11). In particular, miR-10b (12-15) and miR-21 (16-20) appear to be the most highly upregulated oncomiRs contributing to GBM. miR-10b enhances GBM growth by negatively regulating Bim (BCL2 interacting mediator of death), TFAP2C (transcription factor AP-2γ), CDKN2A/16 (tumor suppressor), and p21 (cell cycle inhibitor) expression (13). Inhibiting miR-10b reduces the growth of intracranial GBM tumors in animal models (14, 21). These promising results have prompted the development of an investigational antisense oligonucleotide (RGLS5799, Regulus Therapeutics) targeting miR-10b. Similarly, upregulated miR-21 levels increase GBM invasiveness by inhibiting matrix metalloproteinase (MMP) (17), increase proliferation via negative regulation of insulin-like growth factor binding protein-3 (IGFBP3) (20) or phosphatase and tensin homolog (PTEN), and promote tumor stemness via SOX-2, a transcription factor (22, 23). Hence, knocking down miR-21 reduces GBM progression and invasion (24-26) in addition to preventing chemoresistance of GBM cells to TMZ (27, 28) and taxol (29). Current therapeutic strategies, which are focused on targeting a single oncomiR, have shown limited efficacy against GBM.

In this example, miR-10b and miR-21 were targeted simultaneously to extend survival and to enhance the chemosensitization of GBM towards TMZ. PNAs are synthetic nucleic acid analogues where the phosphodiester backbone is substituted with neutral N-(2-aminoethyl) glycine units (30). PNAs bind to target miRs via complementary Watson and Crick base pairing, but they are enzymatically stable (31, 32). Classical PNAs targeting full length oncomiRs have been explored for cancer therapy (33-35), but the functional activity of miRs is governed by the “seed region” centered on nucleotides 2 to 7 on the 5′ end (36). Hence, antimiR efficacy can be achieved by targeting only the seed region of oncomiRs (37). Here, serine-gamma PNAs complementary to the seed region of oncomiR-21 and oncomiR-10b were designed to achieve improved antimiR activity. Because γPNAs are pre-organized into a helical conformation due to the presence of chirality at γ position, they have superior physicochemical features, binding affinity, and specificity compared to classical PNAs (38-40), making it possible to target short sequences with high affinity. Short anti-seed γPNAs (sγPNAs) possess numerous appealing features: synthesis is straightforward, quality control analysis is simplified over longer sequences, and they are well suited for conjugation with fluorophores or other entities to enable imaging. Most importantly, anti-seed sγPNAs are clinically more translatable and possess comparable in vivo efficacy to conventional full-length anti-miRs with minimal toxicity (37, 41-43).

Over the past years, convection-enhanced delivery (CED) has been an approach for delivery of agents directly to brain tumors, for example, introducing NPs that are loaded with agents directly into the brain (44). NPs composed of a block copolymer of poly (lactic acid) and hyperbranched polyglycerol (PLA-HPG), which can be surface functionalized to introduce aldehyde groups (45), creating PLA-HPG-CHO, have several advantages for delivery of PNA anti-miRs. NPs formed from PLA-HPG-CHO are bioadhesive (46-49). It has been shown that-compared with several other NPs of similar composition-NPs of PLA-HPG-CHO lead to the highest levels of uptake into intracranial tumor cells after CED (50) and they can be loaded with PNAs, which are slowly released after CED in the brain (51). In this example, the PLA-HPG-CHO NPs were loaded with two sγPNAs-one binding to miR-10b and another binding to miR-21. As demonstrated, simultaneous delivery of two anti-miRs in glioma cells can regulate unique molecular pathways leading to improved survival.

### Results

### Design and Synthesis of PNA Oligomers

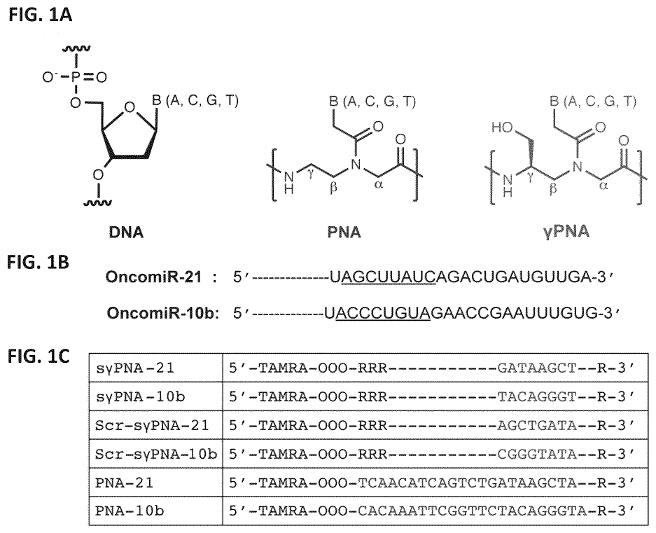

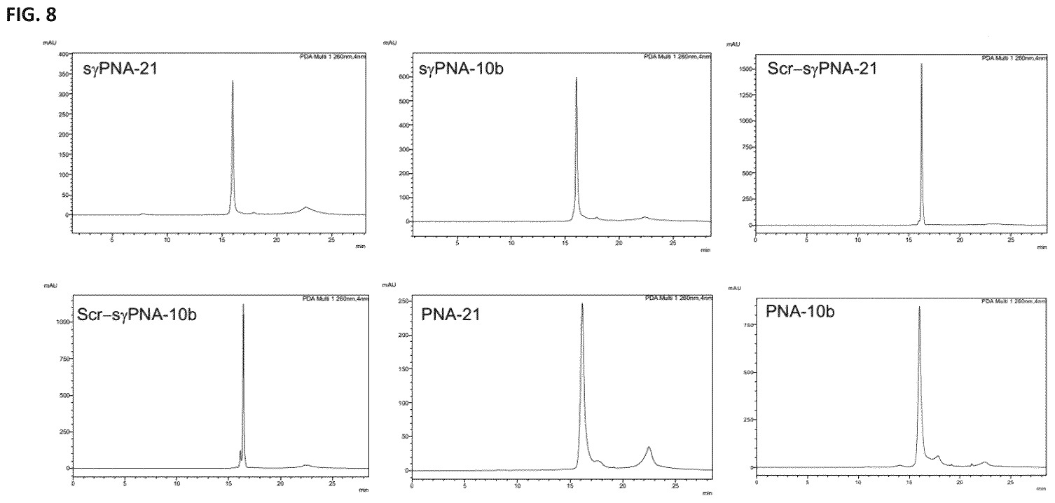

Regular and serine-γPNA oligomers targeting oncomiR-21 and oncomiR-10b were synthesized on a solid support using standard Boc chemistry protocols (FIGS. 1A-1C) (52). sγPNA-21 and sγPNA-10b are sγPNAs—with a serine modification at the γ-position—designed to bind the seed region of oncomiRs 21 and 10b, respectively. Further, sγPNA-21 and sγPNA-10b were extended with three arginine residues on the N-terminus to increase the binding to their respective targets via electrostatic interaction between the cationic domain of γPNAs and the negatively charged backbone in the flanking region of miRNA (53, 54). Scrambled versions of these PNAs Scr-sγPNA-21 and Scr-sγPNA-10b-were used as controls. To compare the efficacy of short cationic γPNAs with full length regular PNAs, PNA-21 and PNA-10b were also synthesized, which were designed to target full length oncomiRs 21 and 10b, respectively (FIGS. 1A-1C). TAMRA dye (5-carboxytetramethylrhodamine) was conjugated to the N-terminus of the PNAs for visualization of cellular uptake and biodistribution. Reverse phase high performance liquid chromatography (RP-HPLC) and mass spectrometry analysis confirmed the high quality of the synthesized PNA preparations (FIG. 8 and Table 1).

TABLE 1
Molecular weight (MW) of perfect match PNAs used in
the study. MW was determined by MALDI-TOF analysis.

| | Calculated MW | Observed MW |
|---|---|---|
| PNA | (Daltons, Da) | (Daltons, Da) |
| sγPNA-21 | 3688 | 3693 |
| sγPNA-10b | 3704 | 3734 |
| PNA-21 | 6654 | 6680 |
| PNA-10b | 7014 | 7015 |

sγPNAs Bind to the Target oncomiRs with High Affinity and Specificity

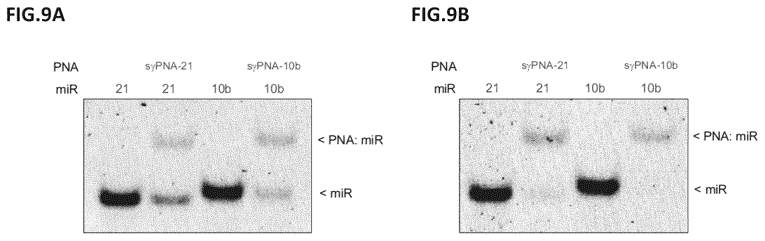

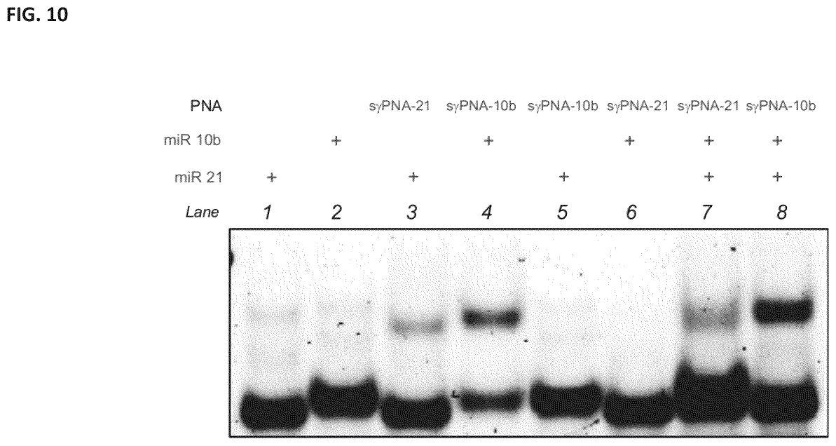

Binding affinity was measured by incubating the PNAs with the target oncomiRs. sγPNA-21 and sγPNA-10b were incubated with miR-21 and miR-10b, respectively, in simulated physiological conditions at two different ratios of 2:1 and 4:1 (PNA:miR) for 16 h. Both sγPNA-21 and sγPNA-10b showed significant binding to their respective targets even at the lower 2:1 ratio (FIG. 9A), as evidenced by the faint band corresponding to the unbound miRs and the prominent retarded band of the PNA-miR heteroduplexes. The band for target miR-21 and miR-10b completely disappeared on incubation with their respective PNAs at 4:1 ratio (FIG. 9B). The binding of sγPNA-21 and sγPNA-10b was also studied in the presence of both miR-10b and miR-21. When incubated with the target miR-21 at 1:1 ratio, sγPNA-21 showed a retarded band (FIG. 10, lane 3). As expected, on incubation of sγPNA-21 with miR 10b, no shift in the band was observed (FIG. 10, lane 6). Incubation of sγPNA-21 with both the miRs resulted in only one retarded band (FIG. 10, lane 7) similar to the sγPNA-21-miR-21 heteroduplex, indicating the specificity of sγPNA-21 towards the target miR-21. Similarly, sγPNA-10b showed a shifted band only after incubation with the target miR-10b (FIG. 10, lane 4) and no retarded band was visible in presence of miR-21 (FIG. 10, lane 5). Further, on incubation with both the miRs, sγPNA-10b showed one retarded band similar to the sγPNA-10b-miR-10b heteroduplex. Hence, sγPNA-21 and sγPNA-10b showed high binding affinity as well as specificity towards miR-21 and miR-10b, respectively.

### Preparation and Characterization of Nanoparticles

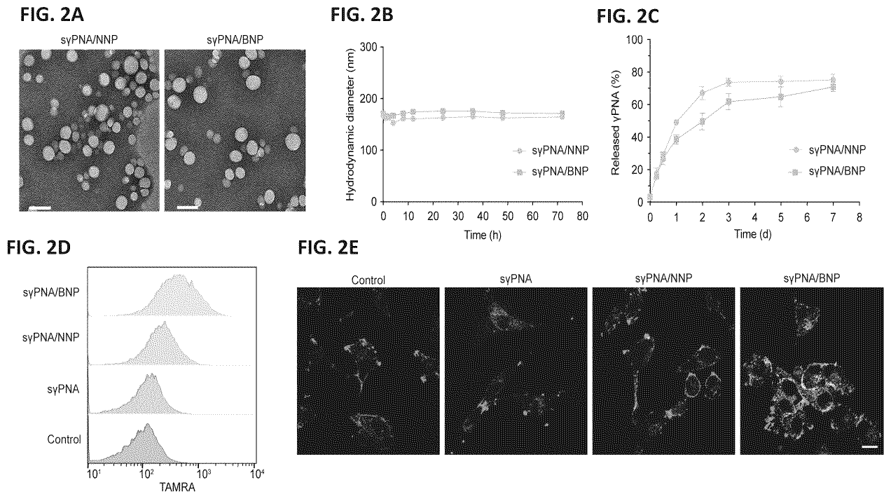

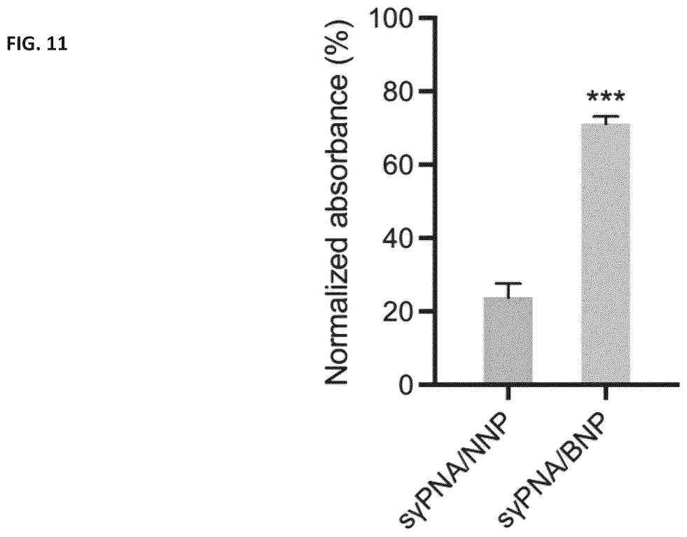

PNAs were encapsulated in PLA-HPG to produce NPs, using a modified single-emulsion method as previously reported (45, 51). Bioadhesive NPs (BNP) were prepared by brief exposure of these NPs to sodium periodate, converting the vicinal diols of HPG to aldehydes, creating HPG-CHO; successful conversion to the bioadhesive state was confirmed by measuring NP adhesion to poly(L-lysine) coated glass (FIG. 11). For the simultaneous knockdown of target oncomiRs 10b and 21, different batches of NPs formulated with respective PNAs were physically mixed at a fixed PNA molar ratio (around 1:1). Single sγPNA loaded NPs and combinations of NP formulations (sγPNA/NNP:sγPNA/PLA-HPG and sγPNA/BNP:γPNA/PLA-HPG-CHO) were extensively characterized (Table 2). sγPNA/NNP and sγPNA/BNP had similar average hydrodynamic sizes, ranging from 150 to 165 nm, as measured by dynamic light scattering (DLS). All the NP formulations exhibited negative surface charge in water with ζ potential between −20 mV to −30 mV. The average PNA loading was around 1.5 nmol per mg of NPs suggesting superior encapsulation efficiency. TEM images showed uniform and spherical morphology of sγPNA/BNP and sγPNA/NNP (FIG. 2A). The majority of visible NPs were around 100 nm in diameter, which confirmed the desired particle size. Both sγPNA/NNP and sγPNA/BNP were stable with no measurable aggregation during 3 days of incubation in artificial CSF (aCSF) (FIG. 2B). In vitro sγPNAs release from both NNP and BNP during continuous incubation in PBS at 37° C. was similar (FIG. 2C), with slightly slower release from BNPs.

TABLE 2
Hydrodynamic diameter, ζ potential and PNA loading
in various nanoparticles. The data are shown as mean ±
SD (n = 3). Scr-sγPNA/NNP was the physical mixture
of Scr-sγPNA-21 loaded NNP and Scr-sγPNA-10b loaded
NNP, sγPNA/BNP was the mixture of sγPNA-21 loaded
BNP and sγPNA-10b loaded BNP, PNA/BNP was the mixture of
PNA-21 loaded BNP and PNA-10b loaded BNP. NNP indicates PLA-
HPG nanoparticles and BNP indicates PLA-HPG-CHO nanoparticles.

| | Hydrodynamic | ζ | PNA |
|---|---|---|---|
| | diameter | potential | loading |
| Formulation | (nm) | (mV) | (nmol/mg) |
| sγPNA-21/NNP | 159 ± 4 | −21 ± 3 | 1.5 ± 0.2 |
| sγPNA-21/BNP | 165 ± 4 | −30 ± 4 | 1.5 ± 0.4 |
| sγPNA-10b/NNP | 150 ± 6 | −21 ± 5 | 1.6 ± 0.3 |
| sγPNA-10b/BNP | 160 ± 5 | −32 ± 3 | 1.4 ± 0.3 |
| Scr-sγPNA/NNP | 176 ± 6 | −22 ± 2 | 1.4 ± 0.2 |
| sγPNA/BNP | 165 ± 3 | −32 ± 3 | 1.5 ± 0.3 |
| PNA/BNP | 171 ± 6 | −28 ± 2 | 1.9 ± 0.2 |

sγPNA Loaded BNP Shows Superior Cellular Uptake in Glioma Cells

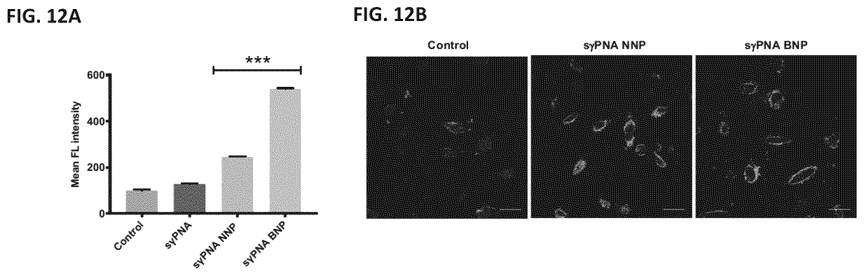

TAMRA fluorescence from sγPNAs was used to quantify the uptake of NPs in glioma cells. Flow cytometry analysis revealed that the uptake of BNP was significantly higher than that of NNP in U87 cells (FIGS. 2D and 12A). Free sγPNAs didn't show a significant uptake after 24 h incubation. For microscopic observation, cells treated with either NNP or BNP showed stronger TAMRA fluorescence than cells treated with free sγPNA. Confocal microscopy (FIG. 2E) indicated that treatment with BNP led to higher cellular uptake than NNP; BNP also produced wider distribution throughout the cells. Similar results were obtained in a different human glioma cell line, LN-229 (FIG. 12B). These findings confirm the preferential cellular uptake of sγPNA/BNP into glioma cells, which is consistent with our previous studies (26, 50).

sγPNA Loaded BNP Inhibit Expression of miR-10b and miR-21

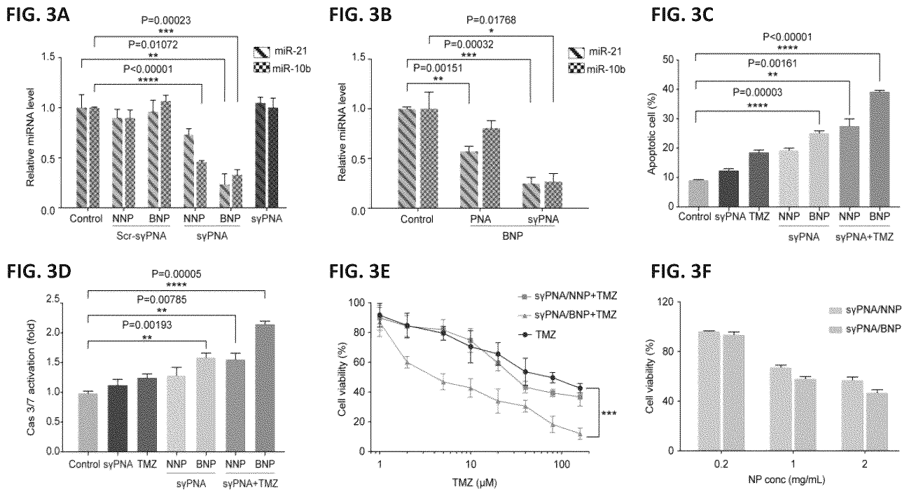

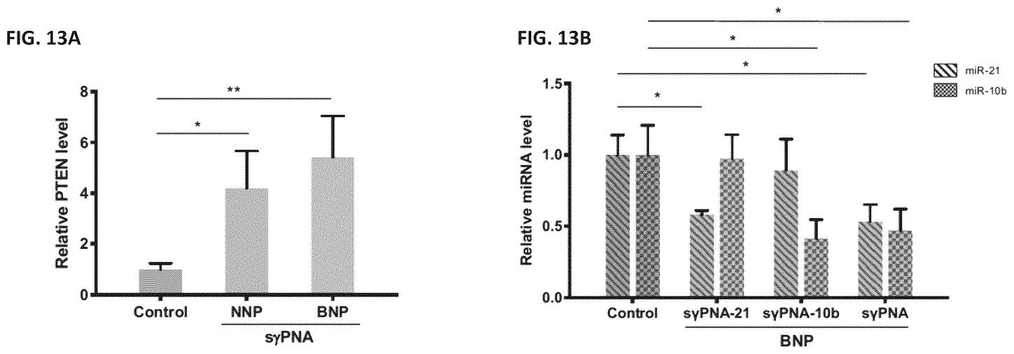

To confirm the therapeutic efficacy of designed anti-miR sγPNAs, different types of PNA-loaded NP formulations or free sγPNAs were incubated with U87 cells. Cellular levels of miR-21 and miR-10b were significantly reduced 72 h after treatment with NPs (FIG. 3A). A significant decrease of miR-10b expression was achieved following incubation with sγPNA/NNP and sγPNA/BNP when compared to the control treatment (FIG. 3A); BNP also produced a significant decrease in miR-21 expression after treatment, while NNP exhibited a slight but not statistically significant effect. Scrambled versions of sγPNA NP formulations and free sγPNAs had no effect on the miR-10b and miR-21 expression (FIG. 3A). Of note, sγPNA/BNP showed greater suppression on both oncomiRs than full length PNA loaded NPs (PNA/BNP), which reflects the higher binding affinity of sγPNAs (FIG. 3B). Also, it was found that PTEN, one of the most frequently mutated tumor suppressor genes in human cancer (55) and a predicted target of both miR-21 (56) and miR-10b (14), was upregulated, by 4-5 fold after treatment with sγPNA loaded NPs (FIG. 13A). Moreover, the specific inhibition of miR-21 mediated by sγPNA-21 loaded NPs did not affect the expression of miR-10b and, likewise, sγPNA-10b loaded NPs did not affect the expression of miR-21 (FIG. 13B). These results demonstrated the specific and effective knockdown of target oncomiRs provided by sγPNA loaded NPs, with BNP providing the strongest biological effect.

sγPNA Loaded BNP Induce Enhanced Apoptosis of U87 Cells in Combination with Temozolomide

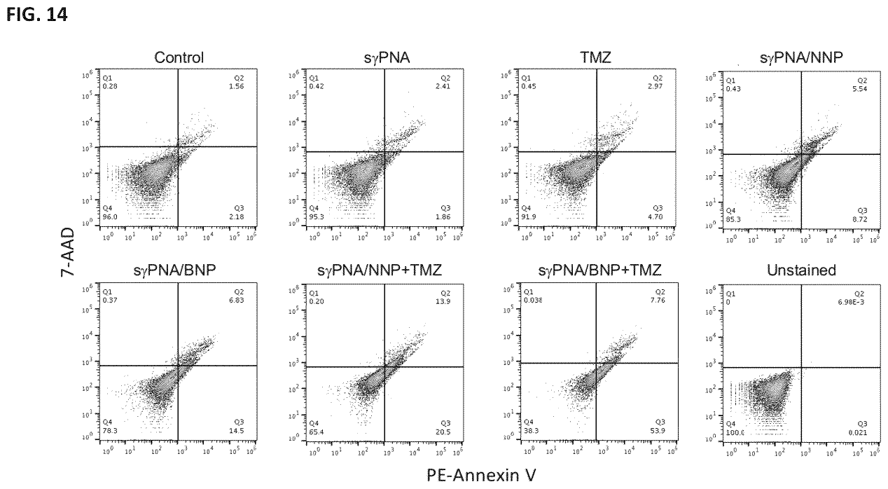

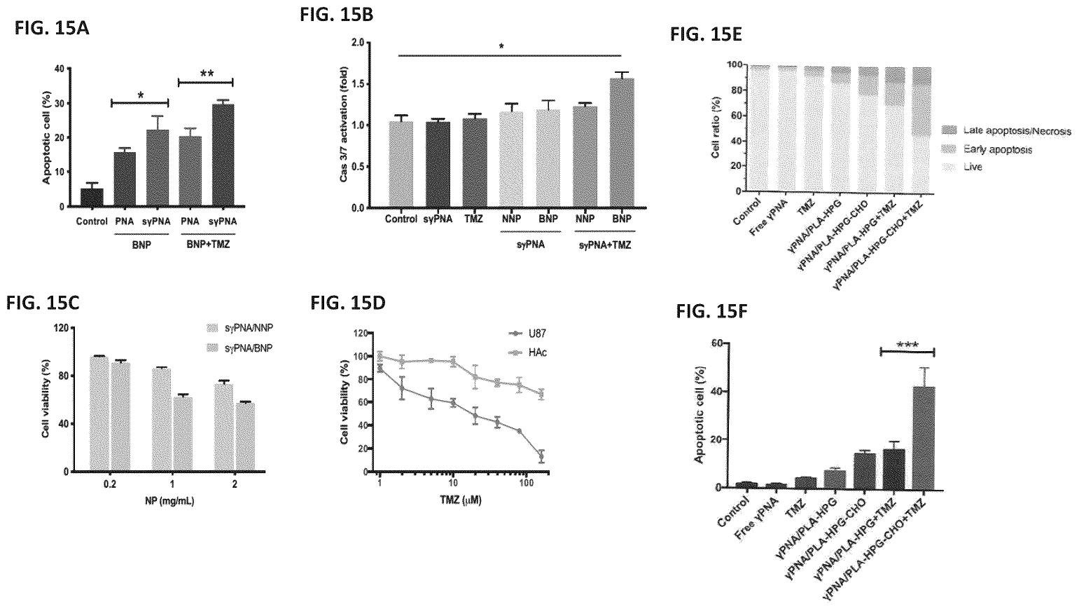

Apoptosis was measured by annexin V and PI staining in cells after treatment with sγPNA loaded NPs. Apoptosis was enhanced by sγPNA/BNP treatment (compared to sγPNA/NNP-treated or control cells), and further enhanced after combined treatment with TMZ (FIG. 3C, FIG. 14). Co-treatment with TMZ and sγPNA/BNP led to higher apoptotic activity than full length PNA BNP (FIG. 15A). These results also showed significant increase in number of apoptotic cells (‘43%) after treatment with TMZ and NPs mixture sγPNA (FIGS. 15E-F). Caspase 3 and caspase 7 activities-key indicators of apoptosis were elevated 2-fold after TMZ and sγPNA/BNP co-treatment (FIGS. 3D and 15B). These results showed a potent apoptotic response of tumor cells following the combination treatment of TMZ and PLA-HPG-CHO NPs mixture.

Tumor Cell Death by Combination Treatment with TMZ

Recent studies show that miRNAs play a role in TMZ resistance, and that regulation of miRNA can enhance TMZ-induced cell death (57). It was found that treatment of U87 cells with sγPNA-loaded NPs sensitized the cells to TMZ (FIG. 3E). Both sγPNA/NNP and sγPNA/BNP showed dose-dependent cytotoxicity against U87 cells (FIGS. 3F and 15C). sγPNA/BNP displayed higher cell killing activity and resulted in up to 54% of cell death after treatment for 72 h, while sγPNA/NNP induced 43% cell death at the same condition. The oncogenic miRNA-targeting combination treatment did not produce significant toxicity on human astrocytes (FIG. 15D).

Simultaneous Inhibition of miR-10b and miR-21 Decreases VEGFA and ITGB8 Levels

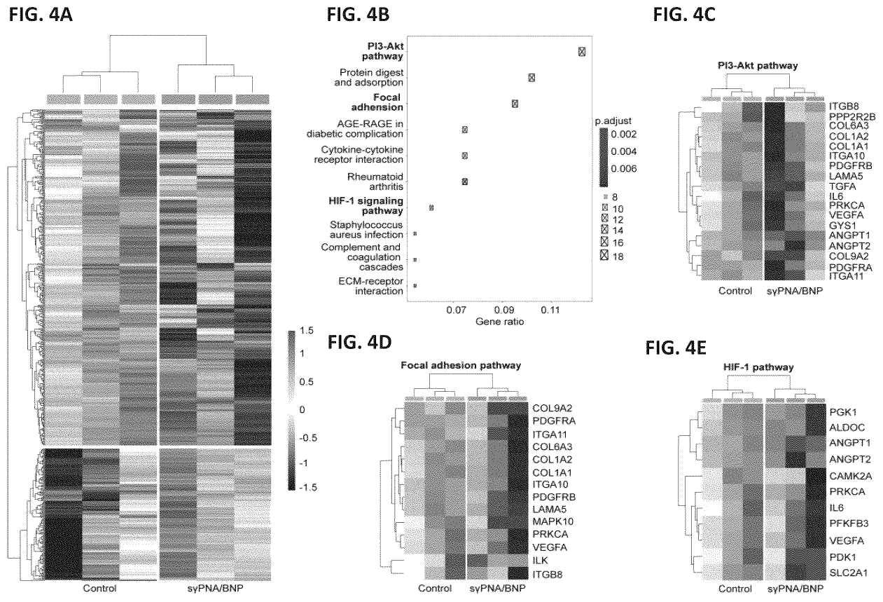

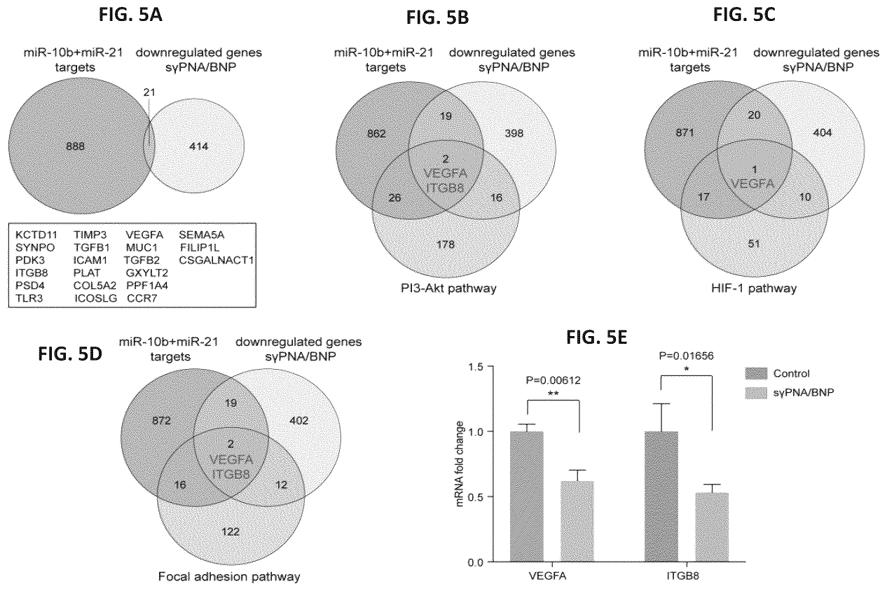

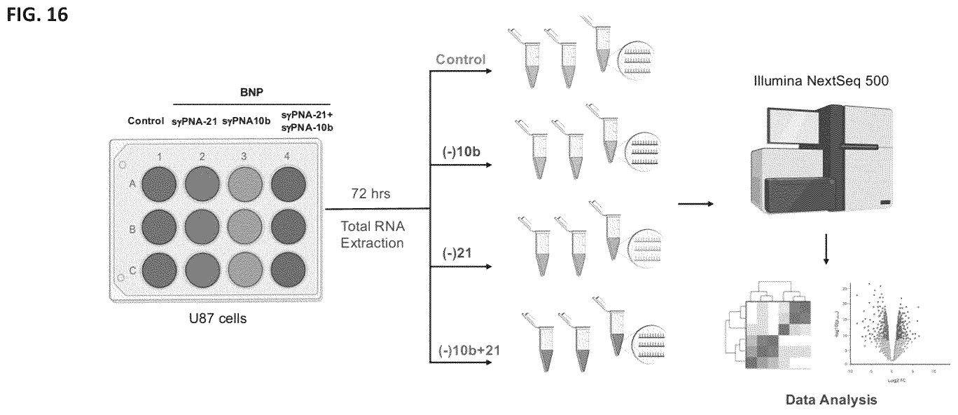

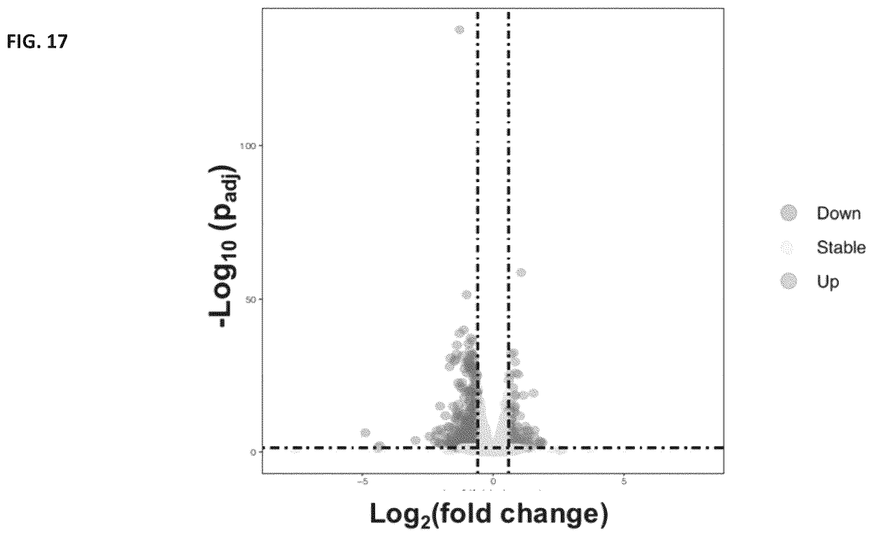

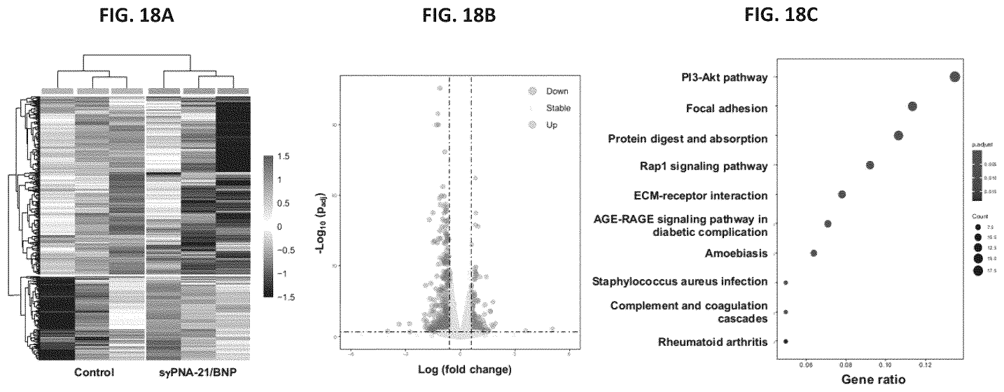

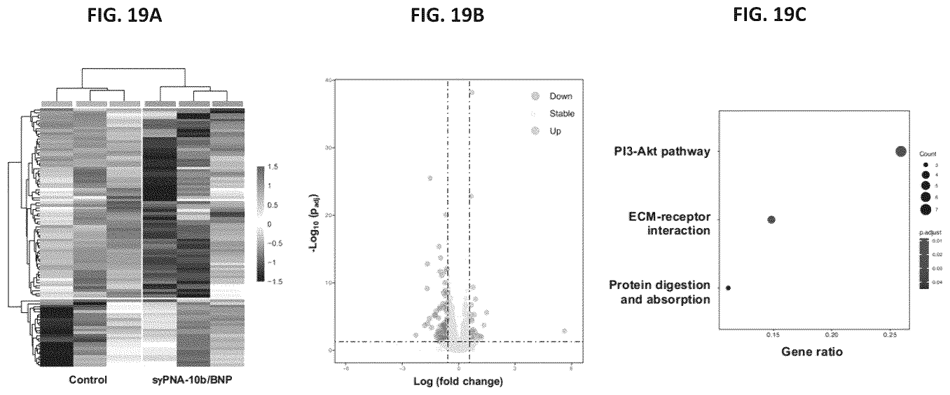

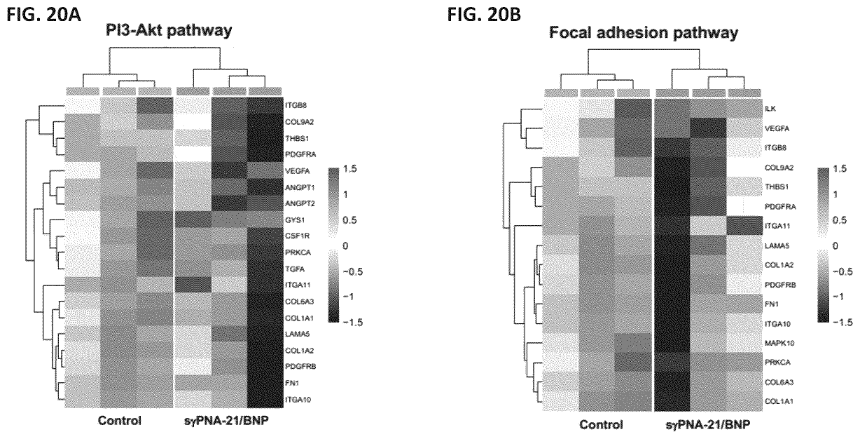

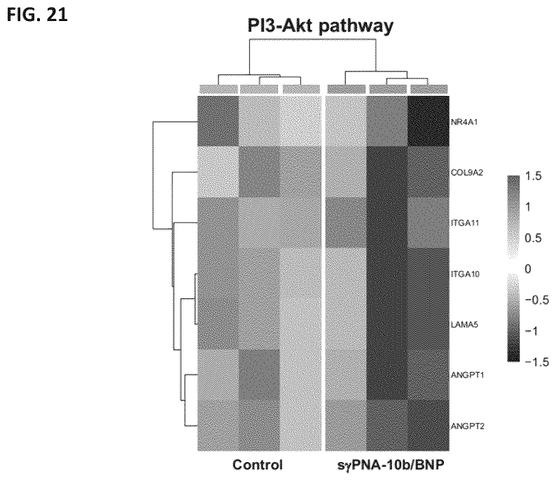

To identify the cellular pathways associated with inhibition of tumor growth, RNA sequencing was performed in sγPNA/BNP treated U87 cells (FIG. 16). Upregulated and downregulated genes were identified in treated U87 cells in comparison to the control, using fold change values of greater than 1.5 with significance (padj) value<0.05 (FIG. 17). The hierarchal clustering of log transformed fold change values of differentially and significantly expressed genes (FIG. 4A) revealed a substantial number of dysregulated genes after knockdown of oncomiRs 21 and 10b in comparison to knockdown of each individual oncomiR (FIGS. 18A-C and 19A-C). Gene ontology analysis in U87 cells after the knockdown of oncomiR 21 indicated enrichment of differentially expressed genes (DEGs) associated with PI3-Akt and focal adhesion pathway (FIGS. 20A-B) while oncomiR 10b knockdown resulted in enrichment of only PI3-Akt pathway (FIG. 21). But the highest enrichment of DEGs was observed in three major pathways; PI3-Akt, HIF-1, and focal adhesion, involved in angiogenesis, proliferation, survival, and metastasis of tumor cells (FIG. 4B) after knockdown of oncomiRs 10b and 21 in U87 glioma cells. Next, the DEGs were isolated in each of the enriched pathways, including PI3-Akt, HIF, and focal adhesion after knockdown of oncomiR 21 and 10b (FIGS. 4C, 4D, and 4E). Further, to isolate the genes associated with GBM pathology and directly regulated targets via miR-10b and miR-21, the observed DEGs were intersected with their predicted targets, i.e., known targets of both miR-10b and miR-21. 21 direct targets of both miR-21 and miR-10b were obtained from the downregulated DEGs identified in the sγPNA/BNP treated group (FIG. 5A). Further intersection of direct downstream targets of miR-10b and miR-21 with the enriched pathways (FIGS. 5B, 5C, 5D) revealed the role of vascular endothelial growth factor A (VEGFA) in all three pathways, i.e., PI3-Akt, HIF-1, and focal adhesion. In addition, integrin beta protein 8 (ITGB8) was also identified as a significant regulator of PI3-Akt and focal adhesion pathway. Further sγPNA/BNP treated U87 cells showed significant downregulation of VEGFA and ITGB8 when validated using qRT-PCR (FIG. 5E).

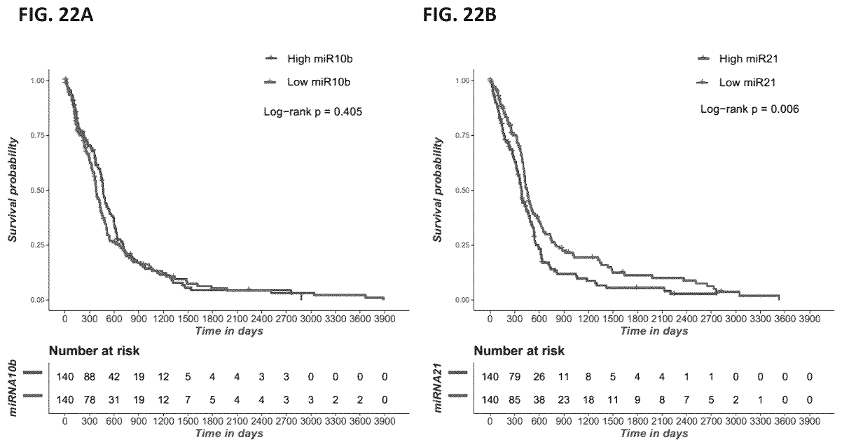

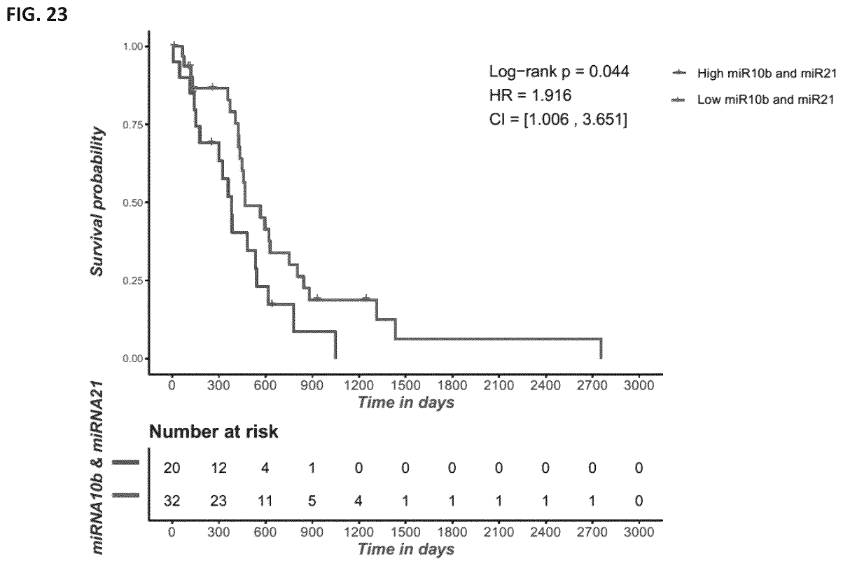

Finally, to connect the findings with GBM in humans, survival of GBM patients was correlated with overexpression of miR-10b and miR-21. Poor survival of patients with higher miR-21 levels was found, but no impact of miR-10b levels on the survival of GBM patients (FIGS. 22A-B). However, when patients with higher expression of both miR-10b and miR-21 were examined, the survival probability reduced to less than half when compared with low levels of miR-10b and miR-21 (FIG. 23). These results indicate that upregulation of both miR-10b and miR-21 contribute towards aggressive growth and poor survival in GBM. Hence, the proposed strategy of targeting multiple oncomiRs can pave way for novel personalized therapeutics for treatment of GBM.

Tumor Retention of sγPNA Loaded BNP after CED

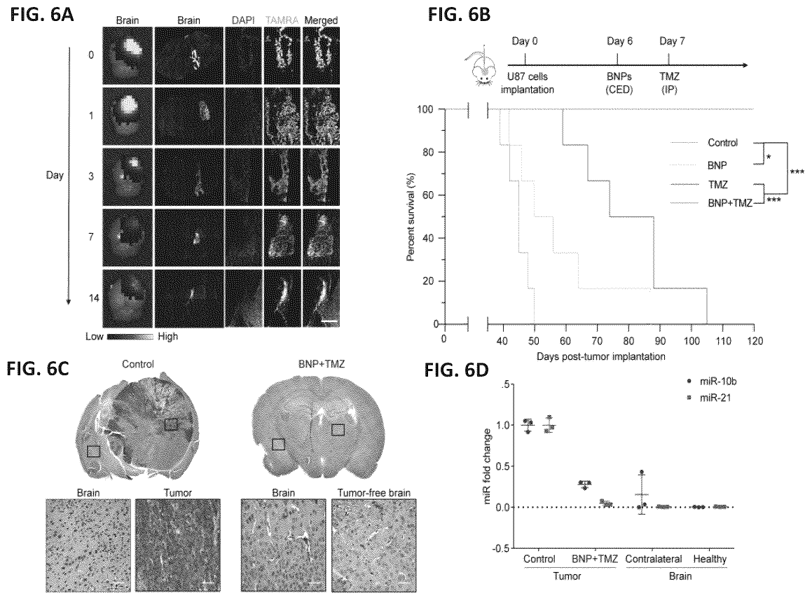

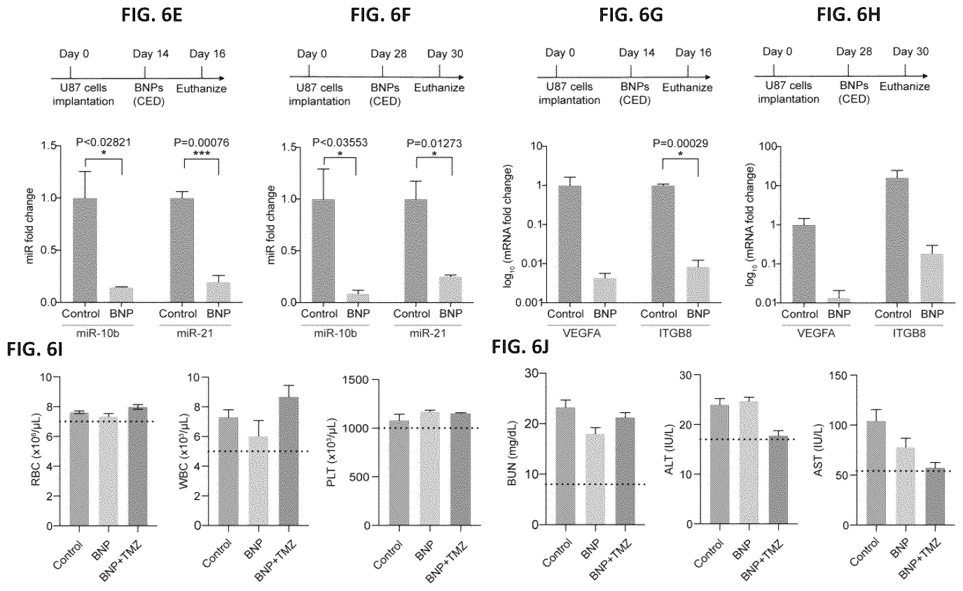

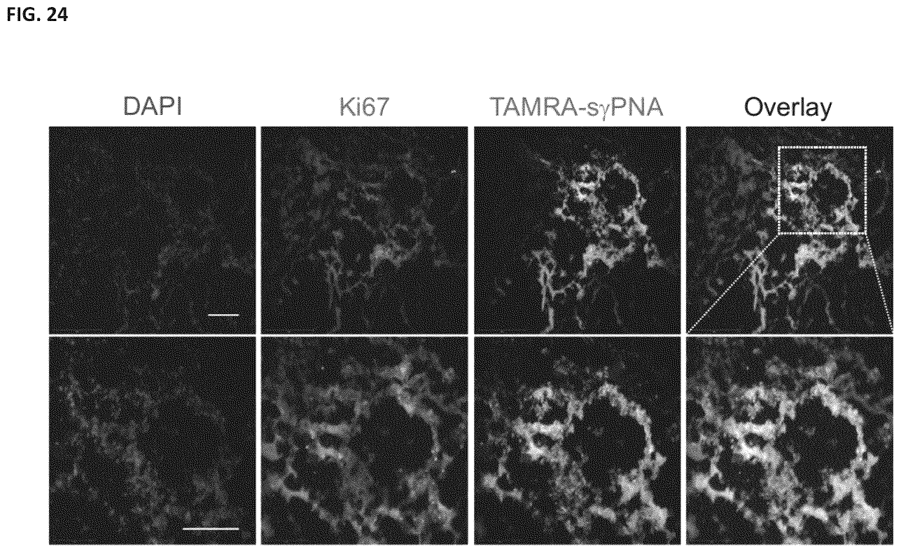

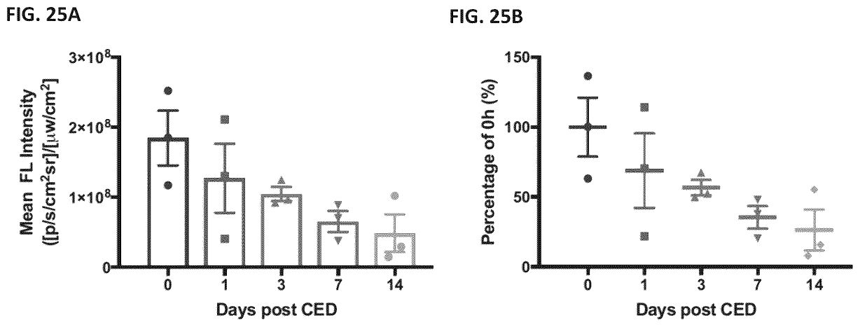

To study tumor retention and biodistribution of sγPNA loaded in BNP after CED, TAMRA-labeled sγPNA/BNP was infused into intracranial tumors and the fluorescence signal of TAMRA-sγPNA was captured at different time points (FIG. 6A). As expected, a strong fluorescence signal was detected in the brain using IVIS 3 h after CED (day 0), and brain cryosection images showed sγPNA/BNP accumulated mainly around the injection site after local delivery. After one day, sγPNA/BNP was observed to distribute more widely from the injection site and spread throughout the tumor region in the brain, as indicated by Ki67, a marker for cell proliferation (FIG. 24). After 7 days, comparatively less fluorescence was detected in the brain, but a visible signal was still retained in the tumor region until day 14, suggesting that s Q PNA molecules were well retained in the tumor for up to two weeks after CED (FIGS. 25A-B). Thus, encapsulation of sγPNA into BNP provided sustained retention within tumor which appears to be suitable for intracranial anti-miR treatment.

### Improved Survival in Orthotopic GBM Tumor Model

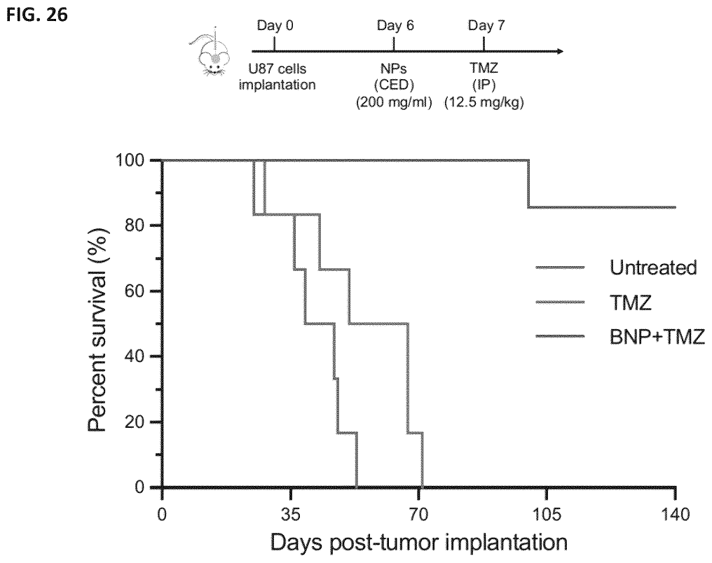

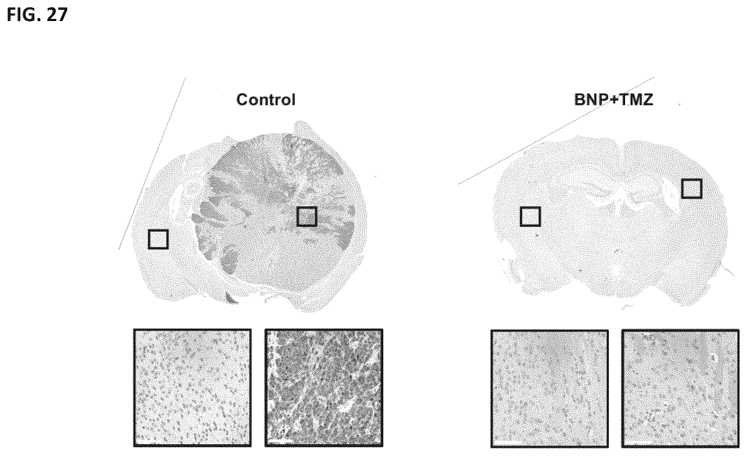

The significant tumor retention provided us a strong rational to evaluate the therapeutic efficacy of the sγPNA/BNP in vivo. Intracranial U87 tumors were generated in immunocompromised mice and NPs were administered via CED 6 days post tumor inoculation (FIG. 6B). The combination of sγPNA/BNP and TMZ was also evaluated by administering a single intraperitoneal injection of TMZ one day after CED infusion. Animals receiving sγPNA/BNP significantly increased median survival to 53 days compared to the untreated control groups (45 days), confirming the oncomiR inhibition mediated by sγPNAs effectively delayed tumor growth. Combination treatment of sγPNA/BNP plus TMZ (25 mg/kg) greatly improved survival and all the animals in this group (n=6) survived over 120 days. At the end of the study, the animals receiving the combination treatment (TMZ plus BNP) appeared to be tumor-free while control animals were found to be hunched due to tumor burden and neurological decline. Administration of TMZ (25 mg/kg) prolonged the median survival to 81 days, confirming therapeutic benefit, but was less effective than the combination with sγPNA/BNP. Further, animals treated by lower doses of TMZ (12.5 mg/kg) did not provide any significant improvement (p=0.0853) in survival comparing to the control group (FIG. 26), while the same dose of TMZ combined with sγPNA/BNP extended survival time over 100 days. These results indicated that sγPNA/BNP in combination with TMZ produced the longest survival time by successfully suppressing tumor growth and reducing drug dose. Analysis of H&E and Ki67 brain sections of one survivor that received combination treatment and one untreated control animal clearly showed the presence of a fully developed tumor localized within the brain of the untreated mouse (FIG. 6C, FIG. 27, left), while the animal in sγPNA/BNP combination treatment group showed no evidence of tumor in the brain (FIG. 6C, FIG. 27, right).

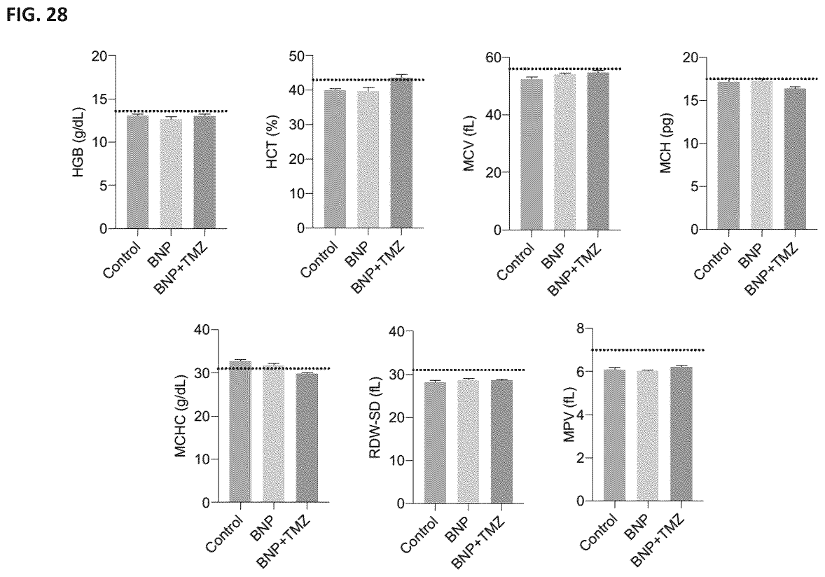

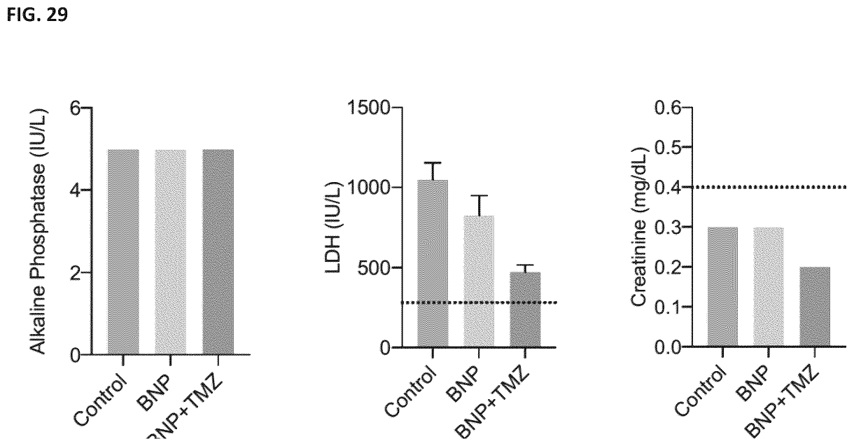

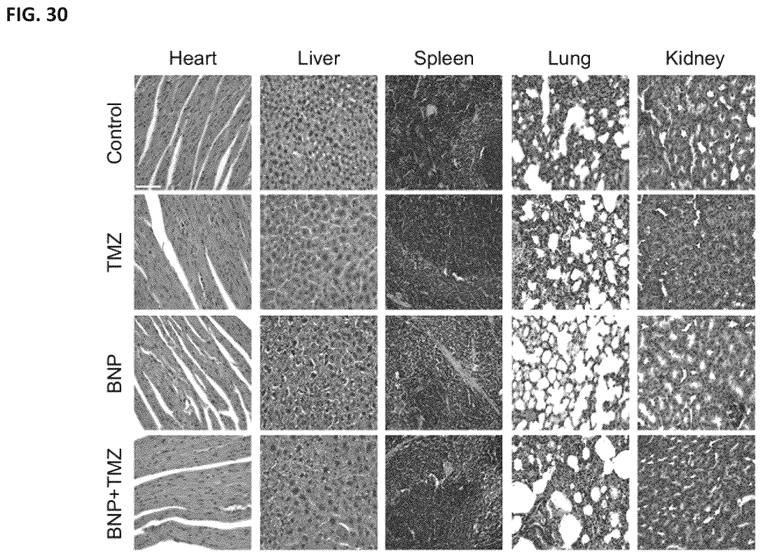

The toxicity of different treatments was also assessed by performing complete blood counts, serum biochemistry, and histopathological analysis. As shown in FIG. 6I and FIG. 28, sγPNA/BNP alone and in combination with TMZ did not alter white blood cell (WBC), platelet (PLT), red blood cell (RBC) or other blood components when compared with control animals. No significant difference was found in liver enzymes (ALT, AST) and renal function markers (BUN) after receiving treatments (FIG. 6J, and FIG. 29). An independently conducted pathological analysis suggested that no obvious tissue changes were found in the treated animals when compared to control animals upon examination of the H&E stained major organs (FIG. 30). Overall, these data confirmed the safety of sγPNA/BNP in combination with TMZ.

In Vivo Knockdown of miR-10b and miR-21

At the end of tumor survival study, the relative levels of miR-10b and miR-21 were measured in the tumors of untreated control animals and brain tissues around the injection site of sγPNA/BNP plus TMZ treatment group (FIG. 6D). Compared to control group, animals in the combined treatment group showed 72% knockdown of miR-10b and 95% of miR-21 in the brain tissues ipsilateral to the injection site, indicating successful oncomiR inhibition mediated by sγPNA/BNP plus TMZ. The expression level of miR-10b and miR-21 in the contralateral hemisphere and healthy mice brains were also measured as comparisons.

To further investigate the role of sγPNA/BNP in oncomiR inhibition, two separate experiments were performed where sγPNA/BNP were administered 14 d post tumor implantation or 28 d post tumor implantation following the treatment schedule indicated in FIGS. 6E and 6F, respectively. Tissues at the tumor site were harvested to examine expression levels of oncomiRs. qPCR results revealed that sγPNA-mediated treatment resulted in statistically significant downregulation of miR-10b and miR-21 in tumors at different growth stages, further confirming efficient suppression of both targets. In addition, the levels of VEGFA and ITGB8 in the sγPNA NP treated animals were evaluated; a consistent downregulation was found compared to control animals (FIGS. 6G-6H).

### Improved Survival in Patient Derived Xenograft (PDX) Mice Model

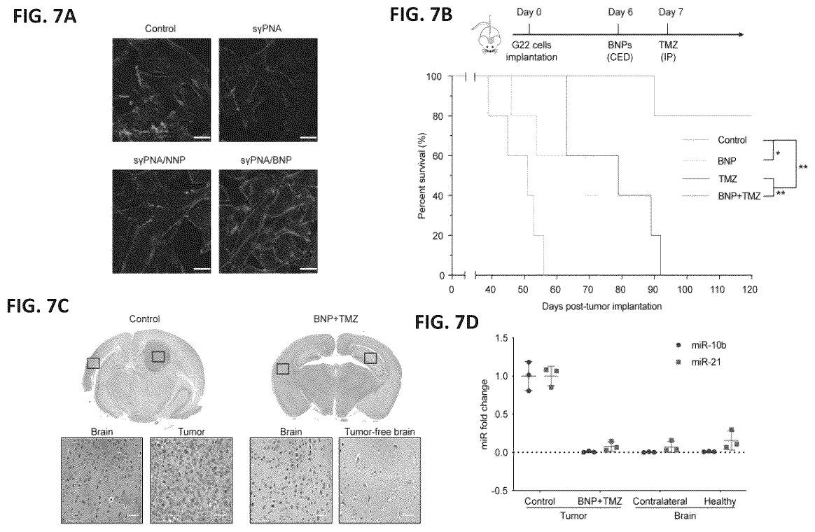

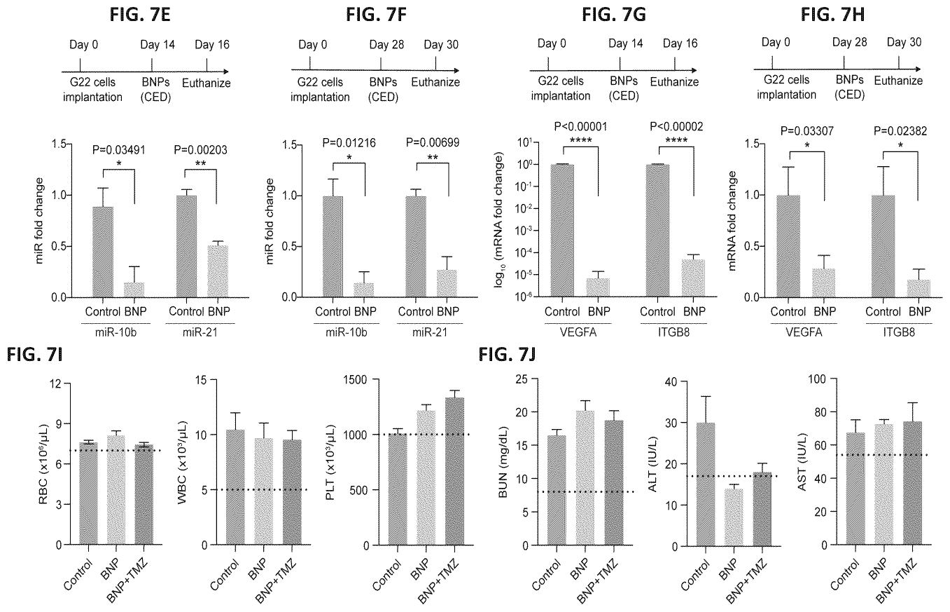

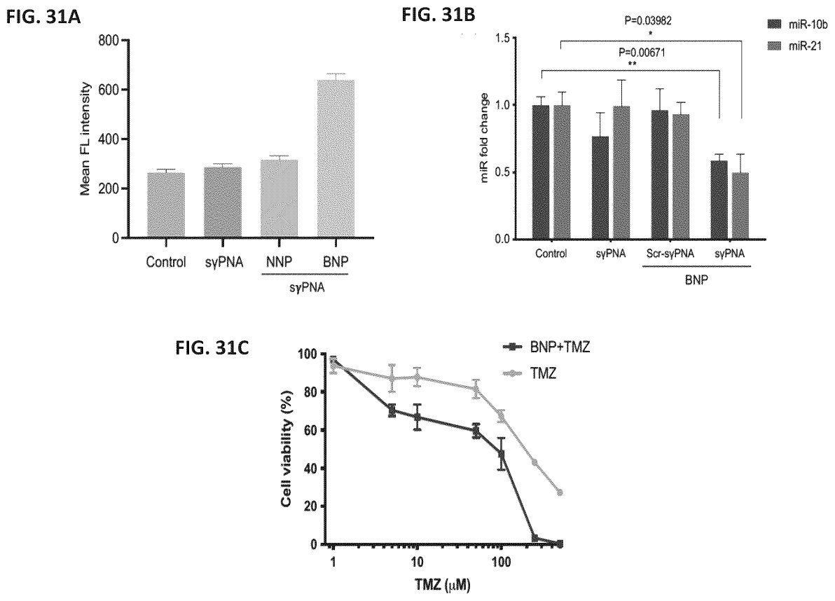

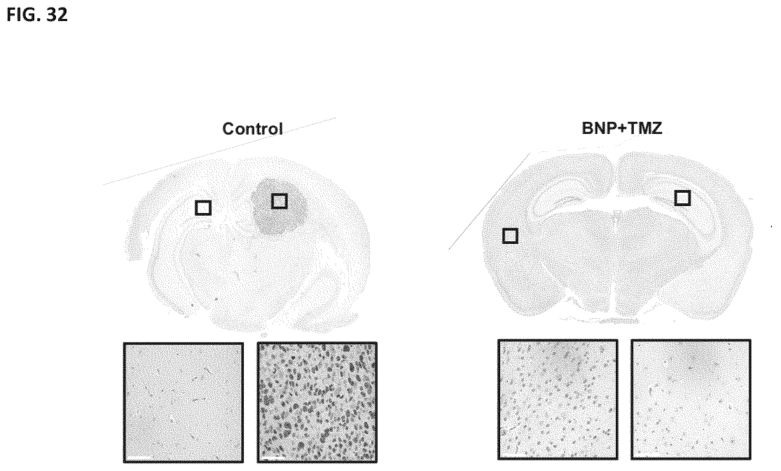

The efficacy and safety of this combination strategy were tested on a patient-derived GBM model (G22) and good cellular uptake of sγPNA-loaded BNP was found in vitro (FIG. 7A, and FIG. 31A). Significant downregulation of miR-10b and miR-21 and elevated response to TMZ treatment were also observed on PDX cells (FIGS. 31B-31C). Patient-derived orthotopic tumor model was established in immunocompromised mice and treatments were performed for further evaluation (FIG. 7B). Similarly, sγPNA/BNP combined with TMZ (25 mg/kg) greatly prolonged survival and 80% of animals survived over 120 days (one died on day 90, n=5). Video clips showed that the animals receiving combination treatment survived till the end of the study with good activity, in comparison untreated control animals showed signs of morbidity as early as day 39 due to tumor development. Histological analysis of H&E and Ki67 stained sections further confirmed free-of tumor brain of BNP plus TMZ treated animal (FIG. 7C and FIG. 32). However, animals receiving sγPNA/BNP treatment or single dose of 25 mg/kg TMZ only extended median survival to 69 days and 79 days compared to the untreated controls (51 days), much less efficient than the combination of sγPNA/BNP plus TMZ.

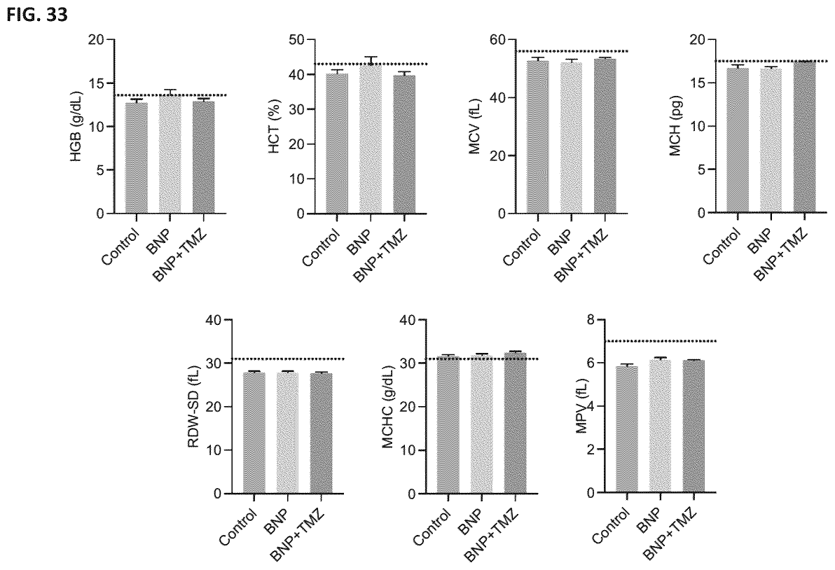

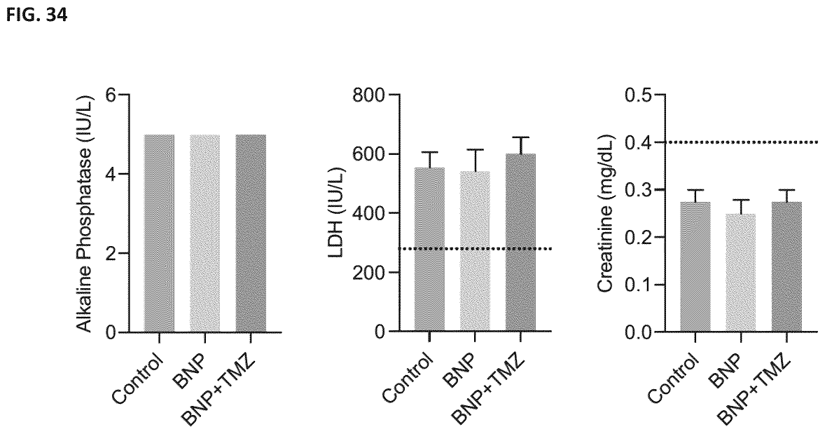

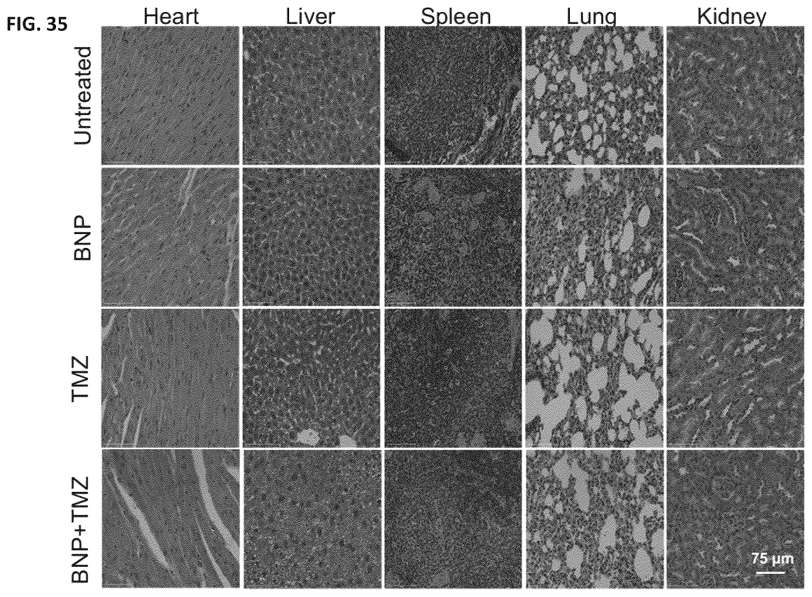

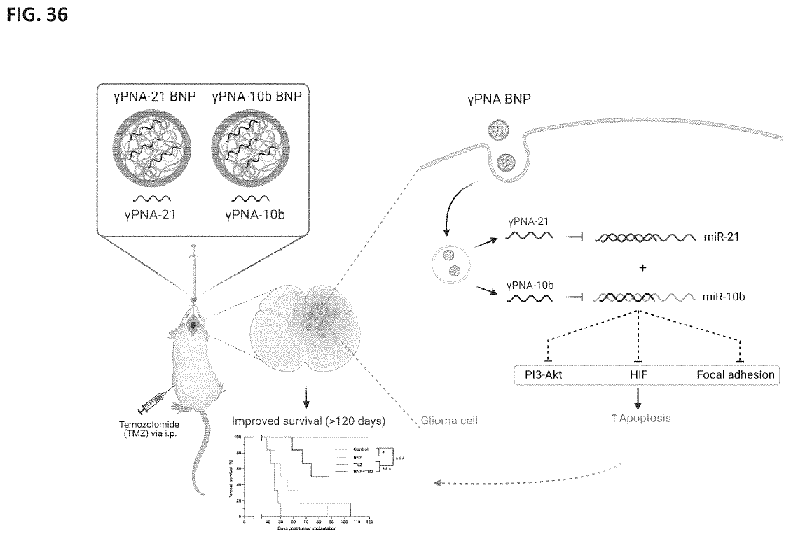

Complete blood counts, serum biochemistry, and histopathological analysis were conducted to evaluate toxicity. FIGS. 7I-7J and FIGS. 32-34 showed no significant difference between the treated animals and controls in terms of blood components including WBC, PLT, RBC and liver enzymes (ALT, AST) and renal function markers (BUN). H&E-stained organ sections of the treated animals didn't appear abnormal comparing with untreated controls (FIG. 35).

In vivo knockdown efficiency was also assessed on PDX model. The relative levels of miR-10b and miR-21 at the end of survival study were found low and comparable to those in the contralateral hemisphere and healthy mice brains (FIG. 7D). 99% of knockdown of miR-10b and 92% of knockdown of miR-21 were observed in the animals treated by sγPNA/BNP combined with TMZ. FIGS. 7E and 7F showed significant downregulation of miR-10b and miR-21 in the tissue at tumor site when administration of sγPNA/BNP was performed 14 days after tumor implantation or 28 days after tumor implantation. Similarly, the expression levels of VEGFA and ITGB8 were significantly lower than the untreated controls (FIGS. 7G-7H). Collectively, sγPNA-loaded BNP resulted in successful inhibition of miR-10b and miR-21 and substantial improvement in animal survival in orthotopic PDX animals, consistent with the findings on intracranial U87 tumors.

### Discussion

Despite significant attention in preclinical and clinical research, GBM remains an aggressive disease, with limited survival and poor treatment options (58, 59). Little progress has been made toward improved survival outcomes in GBM patients over the standard of care, which includes surgical resection, radiation therapy plus temozolomide (TMZ) (60, 61). Reasons for this failure include the lack of powerful therapeutic agents, restricted entry of drugs into intracranial tumors due to blood-brain-barrier (BBB), and tumor heterogeneity driven by multiple coordinated signaling pathways. All of these issues present challenges for GBM therapy (62). Here, the present inventors sought to investigate a potential solution that would involve an addition to the standard of care: infusion of highly effective sγPNA anti-miRs directly into tissue harboring tumor cells, with the sγPNAs packaged into NPs that are highly taken up in tumor cells. This strategy was targeted to two GBM-specific oncomiRs to overcome intrinsic resistance to the induction of cell death.

Several chemically modified oligonucleotides (antimiRs) exhibiting enzymatic stability and high binding affinity have been explored for targeting oncomiRs, including locked nucleic acid (63), 2-0 methyl oligonucleotides (16), morpholinos (64), and PNAs (32). Based on their metabolic stability and binding affinity, PNA oligomers are efficient agents to inhibit the function of microRNAs (65). Modifications of PNAs at the γ-position can address many of the shortcomings of conventional PNAs, further maximizing antagonizing effect on the function of target microRNA (66). Here, short serine-γPNAs conjugated with cationic arginine residues were designed. Gel-shift assays showed that the synthesized sγPNAs (sγPNA-21, sγPNA-10b) bind with miR-10b and miR-21 separately, indicating specific and strong affinity for target oncomiRs. The sγPNAs encapsulated in BNP exhibited a greater miRNA inhibition effect in comparison with non-modified full length PNAs loaded in the same carrier. These short γPNA oligomers have enhanced hybridization due to their γ-modification and cationic residues, and thus, they would improve therapeutic efficacy.

In addition to efficacy, delivery of next generation antimiRs to the CNS remains an enormous challenge that need to be resolved. The efficacy of GBM therapies is significantly limited by the presence of blood-brain-barrier (67), however local delivery approaches have been beneficial to achieve intracranial distribution. Unlike diffusion-based methods (68), CED utilizes positive pressure flow and a catheter to achieve stereotactic placement and direct infusion of drugs into the tumor resection cavity providing large distribution volumes (69). In addition to intracranial delivery, high interstitial distribution can be achieved by adjusting the flow, distribution can be monitored in real time, and anti-tumor efficacy can be achieved at low doses, hence minimizing the neurotoxicity as well as systemic toxicity (70). CED has been employed in multiple clinical trials for intracranial delivery of chemotherapeutics (71), antisense oligonucleotides (72), and liposomal engineered vectors for gene therapy (73). In addition, CED has been investigated at non-clinical and pre-clinical stages for delivery of nanoparticles (74) as well as viral vectors (75) for treatment of gliomas. Of note, recent clinical trials suggest that CED is safe and feasible but the present therapeutic approaches fail to significantly improve survival of GBM (44). This is most likely due to multiple reasons such as technical factors (76) (regions of the brain, catheter placement, rate of infusion), but more importantly, most drugs have short brain half-lives and are eliminated quickly after the infusion stops. Thus, infusion of NPs may improve current CED strategies (77) as NPs offer improved brain retention and sustained drug release for days to weeks after the end of infusion.

To achieve efficient cellular delivery, the sγPNAs were encapsulated in PLA-HPG nanoparticles. Here, 2- and 4-fold increases in cellular uptake were observed with sγPNA loaded NPs formed from PLA-HPG (NNP) or its bioadhesive version, PLA-HPG-CHO (BNP), over free sγPNAs. The preferential association of BNP with tumor cells also resulted in an enhanced oncomiR suppression in vitro analyzed by qRT-PCR. The expression of both miR-10b and miR-21 were significantly downregulated after sγPNA NP treatment. Altered miRNA levels in tumor cells might affect the expression of gene products that stimulate cell proliferation or that induce apoptosis, blocking tumor cells from developing into a proliferative state.

High throughout profiling revealed the overexpression of miR-10b and miR-21 in a large portion of human glioma species (78). Based on the specific molecular aberrations in GBM tumors, sγPNAs were designed targeting these two oncomiRs and formulations loaded with sγPNA oligomers were prepared. Upon simultaneous inhibition of both targeted oncomiRs in vitro, effective cell apoptosis was observed in glioma cells treated by sγPNA/NP. A majority (54%) of apoptotic-associated cell death was achieved by exposure to 2 mg/mL sγPNA/BNP. These results suggested modest anticancer activity mediated by miRNA inhibition in tumor cells. TMZ combined with sγPNA NPs induced a larger degree of cell apoptosis than TMZ or NPs alone. Therefore, the cell killing activity of this combination strategy was evaluated via cell survival assays in vitro, and improved chemosensitivity was speculated in treated tumor cells. As expected, combination with sγPNA/BNP effectively promoted tumor cell responses to TMZ, with significantly reduced cell viability than TMZ alone. Furthermore, this sensitization effect to chemotherapy mediated by γPNA/NP acted in a synergistic manner, as analyzed by the additive model (observed/expected ratio=0.644, Table 3) (79, 80). Tumor cell-specific cytotoxicity was also observed in this combination treatment, which displayed limited toxicity against healthy cells.

TABLE 3
The synergistic effect of gPNA/BNP and TMZ combination treatment
on U87 cells. According to additive model, a ratio between
the observed and the expected viability of tumor cells
was calculated for the combination treatment and a ratio
less than 0.8 was considered to be synergistic.

| Treatment | Cell viability (% of control) | Cell viability/100 |
|---|---|---|
| BNP (0.5 mg/mL) | 80% | 0.8 |
| TMZ (20 μM) | 66% | 0.66 |
| BNP + TMZ | 0.8*0.66 | 0.528 |
| combination-expected | | |
| BNP + TMZ | 34% | 0.34 |
| combination-observed | | |
| Observed/Expected ratio | | 0.644 |

0.644 < 0.8→ Synergism

Limited therapeutic options, the presence of BBB and lack of progress in systemic delivery motivated more direct approaches for GBM therapy, such as local delivery via CED (81, 82). Wide distribution of truly effective agents by CED are expected to influence the overall therapeutic outcomes. With this in mind, CED of sγPNA encapsulated BNP was utilized to improve intracranial drug distribution and prolong the survival of tumor-bearing animals. Up to two-week retention and widespread distribution of sγPNA BNP within the intracranial tumor were observed after CED infusion. This is consistent with the recently reported results where BNPs led to persistent presence of encapsulated camptothecin (˜50%) in the squamous cell carcinoma tumor at 10-day post tumor injection (48). The dramatically improved tumor retentions could be attributed to the enhanced association with tumor cells provided by the bioadhesive surface modifications of aldehyde-rich BNP. Based on the in vitro and in vivo results, BNP-mediated increased tumor cell internalization and sustained drug release bring advantages to localized drug delivery and GBM therapy, particularly for invasive clinical approaches. Moreover, it's important to achieve longer tumor retention when nucleic acid therapies are combined with other approaches (such as chemotherapy, radio therapy) which require multiple doses over time.

In the survival study, CED of sγPNA-loaded BNP plus a single dose of TMZ significantly prolonged survival time of animals bearing intracranial U87 tumors and patient-derived G22 tumors. Although each of the monotherapies, sγPNA/BNP and TMZ alone showed some effect in delaying tumor progression and extending median survival, neither of them was able to completely inhibit tumor growth partially explaining the current limited efficacy of single targeted GBM therapy. Here, all the U87 tumor-bearing animals receiving sγPNA/BNP plus TMZ treatment were tumor-free long survivors (>120 days); this robust antitumor activity could be beneficial for lowering drug dose. For example, sγPNA/BNP combined with a reduced dose of TMZ (12.5 mg/kg) successfully increased survival time of tumor-bearing animals to over 100 days. Similar therapeutic benefits were reproduced on patient-derived GBM tumors thus leading to significantly improved animal survival (P<0.0018, vs control). Suppression of key oncotargets (miR-10b, miR-21) by anti-miR sγPNAs may enhance the sensitivity of glioblastoma cells to chemotherapy, resulting in an elevated response to TMZ treatment.

In conclusion, the inventors of the present invention demonstrated a novel therapeutic approach to deliver anti-miR sγPNA-loaded BNP with bioadhesive surface modifications via CED to intracranial glioblastoma tumors. Two oncomiRs, miR-10b and miR-21, were targeted simultaneously, resulting in cooperative oncomiR inhibition and sensitization of tumors to TMZ treatment. Combined anti-miR sγPNA NPs with TMZ resulted in a remarkable delay of tumor growth and prevention of disease relapse. Thus, the present invention based on available clinical approach may improve eventual glioblastoma therapeutic outcome.

### Material and Methods

Poly (lactic acid) (Mw=20.2 kDa, Mn=12.4 kDa) was purchased from Lactel. Ethyl acetate, acetonitrile and DMSO were obtained from J.T. Baker. Temozolomide (TMZ) was obtained from Enzo Life Sciences. Human glioblastoma cell lines U87 and LN-229 was obtained from ATCC, human astrocyte was kindly provided by Dr. Ranjit Bindra at Yale. G22 (patient-derived xenograft

(PDX) cells) were obtained from Jann Sarkaria (Mayo Clinic, Rochester, MN). The cells were grown in DMEM medium (Invitrogen) supplemented with 10% fetal FBS, 1% Pen-Strep and cultured at 37° C. with 5% C02 in a humidified chamber.

### Synthesis of PNA Oligomers

PNAs were synthesized via solid phase synthesis using MBHA (4-Methylbenzhydrylamine) resin and standard Boc chemistry procedures as reported previously (40). Regular Boc-monomers and serine-γPNA-Boc-monomers (A, T, C, G) purchased from ASM Chemicals and Research (Germany) were used. Three arginine residues were conjugated to N terminus or 5′ end of PNAs. Carboxytetramethylrhodamine (TAMRA) dye, bought from VWR (Pennsylvania, USA), was further conjugated to 5′ end with Boc-MiniPEG-3 linker in between. After completion of synthesis, PNAs were cleaved from the resin using trifluoroacetic acid:trifluoromethanesulfonic acid:thioanisole:m-cresol at a ratio of 6:2:1:1 as cleavage cocktail and precipitated using diethyl ether. PNAs were further purified using RP-HPLC to obtain the pure fraction of PNA. The molecular weight of purified PNAs was confirmed using matrix-assisted laser desorption/ionization-time of flight (MALDI-TOF) spectrometry. The concentration of PNAs in water was determined using UV-Vis spectroscopy. The amount of PNA was then calculated using extinction coefficient of PNA obtained by combining the extinction coefficient of individual monomers of the sequence.

### Gel Shift Assay

The binding of PNAs with the target oncomiRs 10b and 21 was determined by incubating PNAs with target oncomiRs 10b and 21 in a buffer simulating physiological ionic conditions (10 mM NaPi, 150 mM KCl and 2 mM MgCl₂) at 37° C. for 16 hours in thermal cycler (Bio-Rad, USA). The samples were separated using 10% non-denaturing polyacrylamide gel and tris/boric acid/EDTA buffer (TBE) at 120V for 35 minutes. For detection of bound and unbound fraction of target oncomiRs, gels were stained using SYBR-Gold (Invitrogen, USA) followed by imaging in a Gel Doc EZ imager (Bio-Rad, USA).

### Nanoparticle Preparation

sγPNA/NNP. sγPNA loaded NNP were prepared using emulsion-evaporation method as previously reported. Fifty (50) mg polymer (PLA-HPG) was dissolved in 2.4 mL of ethyl acetate overnight. 50 nmol sγPNA was dissolved in 0.6 mL of DMSO and then added to polymer solution, obtaining PNA/polymer mixture. The resulting solution was added dropwise to 2 mL of DI water under a strong vortex, and then sonicated 10 s for 4 cycles. The emulsion was diluted in 20 mL deionized (DI) water and concentrated in a rotovap for 20 min. The particle solution was transferred to an Amicon centrifugal filter unit (100 KDa) and washed twice by water. Lastly, the obtained particles were resuspended in DI water and snap-frozen in aliquots. sγPNA-21 loaded NNP and sγPNA-10b loaded NNP were mixed at 1:1 molar ratio to obtain sγPNA/NNP before further use. Scr-sγPNA and regular PNA loaded NNP were prepared using the same method.

sγPNA/BNP. To create sγPNA/BNP, 25 mg/mL sγPNA/NNP (as above) was incubated with 0.1 M NaIO₄ (aq) and 10×PBS (1:1:1, v:v) for 20 min on ice. 0.2 M NaSO₃ at 1:1 vol ratio was added to quench the reaction. The particle solution was washed with water at 13,000 rcf using a centrifugal filter unit (Amicon, 100 KDa) and resuspended in DI water. Regular PNA loaded BNPs were synthesized with the same method.

### Nanoparticle Characterization

Transmission electron microscopy (TEM). For TEM imaging, 2 μL of particle solution (20 mg/mL) was applied on a CF400-CU grid (Electron Microscopy Sciences) for 1 min. Extra liquid was carefully removed and the grid was stained by one drop NANO-W (Nanoprobe) for 1 min. Liquid was removed and sample was air dried before imaging. Images were obtained using Tecnai Osiris (FEI).

Size and ζ potential. The hydrodynamic diameter of NPs was measured by dynamic light scattering (DLS) using a Malvern Nano-ZS (Malvern Instruments). NPs were diluted to 0.2 mg/mL with DI water before measurement. The same particle solution was loaded into a disposable capillary cell to measure (potential on the Malvern Nano-ZS.

Size stability in aCSF. Particle solutions were incubated in artificial cerebrospinal fluid (aCSF, Harvard Apparatus) at 37° C. and measured by DLS at designated time points.

sγPNA loading and release. 100 μL of particle solution was lyophilized in a pre-weighed tube to measure NP yield. Following lyophilization, NPs were dissolved in acetonitrile and incubated for 24 h at room temperature. Absorbance at 260 nm was read by a Nanodrop 8000 (Thermo Fisher) to measure sγPNA loaded in the NPs. Scr-sγPNA and regular PNA loading efficiencies were determined using the same method. Release profile of sγPNA from different NP formulations was analyzed by incubating 10 mg NPs in 1 mL PBS (pH 7.4) in a shaking incubator at 37° C. At predetermined time points, aliquots were taken out and centrifuged using Amicon centrifugal filter unit (100 KDa). Filtrates were collected for analysis.

Cellular Uptake of sγPNA Loaded Nanoparticle

Cells were seeded in 24-well plates at a density of 50,000 cells/well and incubated with either 0.5 mg/mL NP or same concentration of free sγPNA for 24 h. sγPNA oligomers used in this experiment were labeled with TAMRA for fluorescent evaluation. The cells were harvested, and cell uptake was determined from TAMRA fluorescence per cell using Attune NxT (Invitrogen) flow cytometer and FlowJo software for data analysis. For microscopic observation, U87 cells were cultured in a 20 mm glass-bottom dish (20,000 cells/dish) before treatments. Cells were exposed to NPs (1 mg/mL) or free sγPNA for 24 h and washed by PBS after treatments. After 4% paraformaldehyde fixation, cells were stained with Alexa Fluor 488 phalloidin (Life Technologies) and DAPI, and observed by SP5 confocal microscope (Leica).

Quantitative Real-Time Polymerase Chain Reaction (qRT-PCR)

The knockdown of miR-10b and miR-21 were analyzed by qRT-PCR. Cells were seeded in 24-well plates at a density of 200,000 cells/well. Cells were treated with various formulations at a PNA concentration of 300 nM. Scr-sγPNA loaded NPs and regular PNA loaded NPs with the same total PNA concentration were applied as control groups. After 72 h, total RNA was extracted using the mirVana miRNA Isolation Kit (Ambion). cDNA synthesis was performed using TaqMan Advanced miRNA cDNA Synthesis Kit (Thermo Fisher). PCR reactions were performed with TaqMan Fast Advanced Master Mix (Thermo Fisher), and TaqMan Advanced miRNA Assays (Thermo Fisher) for miR-10b, miR-21 and miR-26b analysis. MiRNA levels were quantified using CFX Connect Real-Time PCR Detection System and CFX Manager Software (Bio-Rad). Relative expression was calculated according to the comparative threshold cycle (Ct) method and normalized by miR-26b. For evaluation of PTEN mRNA level, PCR reactions were performed using PTEN and GAPDH TaqMan Gene Expression Assays (Thermo Fisher) and quantified with CFX Real-Time PCR Detection System and CFX Manager Software. The results were calculated with Ct method and normalized by GAPDH.

### Annexin V Assay

Apoptosis was assessed using the FITC Annexin V Apoptosis Detection Kit (BD Pharmingen). Briefly, tumor cells were plated in 24-well plates at a density of 200,000 cells/well. The cells were treated with free sγPNA, sγPNA loaded NPs and/or TMZ for 72 h (200 nM sγPNA-21, 200 nM sγPNA-10b, 40 μM TMZ). The FITC Annexin V apoptosis detection was performed using flow cytometry in accordance with the manufacturer's protocol, and the data were processed using FlowJo. FITC-Annexin V positive and PI-negative cell populations were identified as early apoptotic cells. Cells that stain positive for both FITC Annexin V and PI are either in the end stage of apoptosis or are undergoing necrosis. Cells that stain negative for both dyes are identified as alive.

### Caspase 3/7 Activity Evaluation

U87 cells were plated in 96-well plates at a density of 5,000 cells/well and treated with various sγPNA nanoparticle formulations and TMZ (200 nM sγPNA-21, 200 nM sγPNA-10b, 40 μM TMZ). Treatments were removed 48 or 72 h later and enzymatic activities of caspase-3 and -7 were measured using Caspase-Glo 3/7 Assay (Promega) and read by a microplate reader (SpectraMax M5).

### Cell Viability Assay

Cells were seeded in 96-well plates at a density of 5,000 cells/well and treated with increasing concentrations of NP formulations. Cell viability was evaluated by CellTiter-Glo Luminescent Cell Viability Assay (Promega) after 48 h, 72 h treatment. Luminescence was measured using a plate reader (SpectraMax M5) and relative cell viability was normalized to the viability of untreated cells. For TMZ involved combination studies, cells were seeded in 96-well plates at a density of 1,000 cells/well and exposed to sγPNA/NNP and sγPNA/BNP. After 72 h of treatments, NPs were removed and TMZ was added to the wells. Cell viability was measured as described above after 6 days of treatment.

### RNA-Sequencing

U87 cells were seeded in 24-well plates at a density of 50,000 cells/well and cultured overnight before use. sγPNA-21/BNP (150 nM sγPNA-21), sγPNA-10b/BNP (150 nM sγPNA-10b), sγPNA/BNP (150 nM sγPNA-21, 150 nM sγPNA-10b) were added to cells and incubated for 72 h. Total RNA from each sample was extracted using the mirVana miRNA Isolation Kit. The libraries were made using Illumina TruSeq Stranded mRNA library preparation. The sequencing was done using Illumina NextSeq 500.

Analysis of RNA-Sequencing Data and miRNA Targets

Total counts per gene were quantified and used in further analysis. All downstream analyses were accomplished by R (3.6.3). Differentially expressed genes (DEGs) between different groups were identified by the package DESeq2 (1.26.0) with a filtering criteria of fold change (FC)>1.5 and adjust p value (padj)<0.05(83). The package cluster profiler (3.14.3) was used to identify specific pathways overrepresented in the DEGs, and significant pathways were picked out by setting pvalue cutoff=0.05 and qvalue cutoff=0.05 (84). Because the miRTarBase2020 is the updated version of the experimentally validated microRNA-target interaction database (85), the targets of miR10b-5p and miR21-5p were downloaded from the website (http://miRTarBase.cuhk.edu.cn/) and were used in the study.

### TCGA GBM Data Analysis

The TCGA miRNA expression level-3 data and metadata containing survival information for GBM patients were downloaded from http://gdac.broadinstitute.org/. The GBM patients were ranked from high to low according to their miR 10b or miR 21 expression level, then labeled the top 25% patients as the miR-higher group, bottom 25% ones as the miR-lower group. One GBM patient would be marked as miR 10b & miR 21-higher when this patient was in both miR 10b-higher group and miR 21-higher group, and miR 10b & miR 21-lower patient was in both miR 10b-lower group and miR 21-lower group. Survival curves were performed by Kaplan-Meier analysis between miR higher and lower group, and were tested for significance using the Mantel-Cox log-rank test. A value of p<0.05 was considered statistically significant. Between miR 10b & miR 21-higher and miR 10b & miR 21-lower group, hazard ratio (HR) and confidence interval (CI) were also computed by function coxph in package survival (3.2-7).

In Vivo Study of sγPNA/BNP

All procedures were approved by the Yale University Institutional Animal Care and Use Committee (IACUC) and performed in accordance with the guidelines and policies of the Yale Animal Resource Center (YARC). Athymic nude mice (Charles River Laboratories, 6-7 weeks) were used for animal study.

### Orthotopic Tumor Inoculation

Animals were anaesthetized using a mixture of ketamine (100 mg/kg) and xylazine (10 mg/kg) via intraperitoneal injection. Anesthetized animals were then placed in a stereotaxic frame and sterilized the scalp with betadine and alcohol. To expose the coronal and sagittal sutures, a midline scalp incision was created, and a burr hole was drilled 2 mm lateral to the sagittal suture and 0.5 mm anterior to the bregma. U87 or G22 cells (3.5×105) in 3 μL PBS were injected into the right stratum over 3 min using a 10-μL Hamilton syringe. The animal was left 5 min for tissue equilibration before and after infusion. When infusion was finished, the burr hole was filled with bone wax and skin was stapled and cleaned.

Convection-Enhanced Delivery of sγPNA/BNP in the Tumor Bearing Brain

CED in tumor bearing mice was similar to tumor inoculation by reopening the burr hole used for tumor inoculation. A micro-infusion pump (World Precision Instruments) was used to infuse 6 μL of NPs at a rate of 0.5 L/min.

Tumor Retention of sγPNA/BNP

Intracranial CED of sγPNA/BNP was conducted 10 days after tumor implantation using the same procedure as previously described. On day 0, 1, 3, 7 and 14 after CED administration, mice were sacrificed and brains were harvested and imaged using Xenogen IVIS. The fluorescence from each brain was quantified by the instrument software. Then, the isolated brains were embedded in an OCT compound, cut into 15 microns frozen sections, stained with DAPI or H&E (hematoxylin and eosin) and observed using a EVOS microscope (FL Auto 2).

### Survival Study in the Tumor Bearing Brain

Tumors grew for 6 days before the administration of treatment. Intracranial CED of sγPNA/BNP was conducted following the same surgical procedure as described. TMZ (25 mg/kg) in PBS was administered intraperitoneally on day 7. Animals were monitored daily and weighed every week. Animals were euthanized once they showed clinical symptoms of tumor progression or greater than 15% weight loss. Tumors and major organs were harvested and fixed in 4% paraformaldehyde and sectioned for histochemical analysis. Total RNA in the tumor and contralateral hemisphere was isolated using mirVana miRNA Isolation Kit and analyzed by real-time PCR detection system as previously described.

Evaluation of miR-10b and miR-21 Inhibition in Tumor

To assess in vivo knockdown effect, sγPNA/BNP were administered by CED 14 d or 28 d post tumor inoculation. Two days after CED, mice were sacrificed and brains were harvested for RT-qPCR analysis. Tumor tissue was separated from the adjacent normal brain areas of isolated brains. Total RNA was extracted from tumor tissue using the mirVana miRNA Isolation Kit (Ambion). MiRNA levels were quantified using CFX Connect Real-Time PCR Detection System and CFX Manager Software (Bio-Rad) as described.

### Evaluation of VEGFA and ITGB8

The levels of VEGFA and ITGB8 were quantified in total RNA samples extracted from treated U87 cells in vitro and in vivo tumor samples. The cDNA was synthesized using a high capacity cDNA reverse transcription kit (Thermo Fisher). The mRNA levels of VEGFA and ITGB8 were quantified using TaqMan gene expression assays (VEGFA: Hs00900055, ITGB8: Hs001744546) (Thermo Fisher) and TaqMan fast advanced master mix (Thermo Fisher) on CFX connect Real-Time PCR Detection system (Bio-Rad). GAPDH was used as reference and relative fold change was calculated using the ct method.

### Toxicity Study

For evaluation of the toxicity, tumor-bearing mice were administered of sγPNA/BNP via CED 14 d post tumor inoculation. After 24 h, TMZ (25 mg/kg) was injected intraperitoneally. Forty-eight (48) h post CED, blood from the retro-orbital venous plexus of each mouse was collected in EDTA tubes. The whole blood was analyzed using Sysmex XP-300 hematological analyzer (Sysmex) to obtain the complete blood count. Plasma was isolated from blood samples via centrifugation at 4500 rpm and 4° C. for 10 minutes. Plasma samples were then analyzed by Antech Diagnostics to obtain the blood biochemistry analysis including LDH, AST, ALT, creatinine, and BUN. Major organs (heart, liver, spleen, lung, kidney) were isolated and sectioned for H&E and Ki67 staining. Blinded histological analysis of the tissue was conducted by pathologist at Yale medical school.

### Statistical Analysis

Data are presented as mean±SD or SEM. Statistical significance was performed with Prism software (GraphPad) using one-way ANOVA. p<0.05 as the minimal level of significance.

### REFERENCES

1. A. M. Molinaro, J. W. Taylor, J. K. Wiencke, M. R. Wrensch, Genetic and molecular epidemiology of adult diffuse glioma. Nat Rev Neurol 15, 405-417 (2019).
2. F. G. Davis, T. R. Smith, H. R. Gittleman, Q. T. Ostrom, C. Kruchko, J. S. Barnholtz-Sloan, Glioblastoma incidence rate trends in Canada and the United States compared with England, 1995-2015. Neuro Oncol 22, 301-302 (2020).
3. J. A. Schwartzbaum, J. L. Fisher, K. D. Aldape, M. Wrensch, Epidemiology and molecular pathology of glioma. Nat Clin Pract Neurol 2, 494-503; quiz 491 p following 516 (2006).
4. R. Stupp, W. P. Mason, M. J. van den Bent, M. Weller, B. Fisher, M. J. Taphoorn, K. Belanger, A. A. Brandes, C. Marosi, U. Bogdahn, J. Curschmann, R. C. Janzer, S. K. Ludwin, T. Gorlia, A. Allgeier, D. Lacombe, J. G. Cairncross, E. Eisenhauer, R. O. Mirimanoff, R. European Organisation for, T. Treatment of Cancer Brain, G. Radiotherapy, G. National Cancer Institute of Canada Clinical Trials, Radiotherapy plus concomitant and adjuvant temozolomide for glioblastoma. N Engl J Med 352, 987-996 (2005).
5. L. He, G. J. Hannon, MicroRNAs: small RNAs with a big role in gene regulation. Nat Rev Genet 5, 522-531 (2004).
6. C. J. Cheng, W. M. Saltzman, F. J. Slack, Canonical and non-canonical barriers facing antimiR cancer therapeutics. Curr Med Chem 20, 3582-3593 (2013).
7. A. Esquela-Kerscher, F. J. Slack, Oncomirs—microRNAs with a role in cancer. Nat Rev Cancer 6, 259-269 (2006).
8. P. P. Medina, M. Nolde, F. J. Slack, OncomiR addiction in an in vivo model of microRNA-21-induced pre-B-cell lymphoma. Nature 467, 86-90 (2010).
9. F. J. Slack, J. B. Weidhaas, MicroRNA in cancer prognosis. N Engl J Med 359, 2720-2722 (2008).
10. S. Volinia, G. A. Calin, C. G. Liu, S. Ambs, A. Cimmino, F. Petrocca, R. Visone, M. Iorio, C. Roldo, M. Ferracin, R. L. Prueitt, N. Yanaihara, G. Lanza, A. Scarpa, A. Vecchione, M. Negrini, C. C. Harris, C. M. Croce, A microRNA expression signature of human solid tumors defines cancer gene targets. Proc Nat Acad Sci USA 103, 2257-2261 (2006).
11. S. A. Ciafre, S. Galardi, A. Mangiola, M. Ferracin, C. G. Liu, G. Sabatino, M. Negrini, G. Maira, C. M. Croce, M. G. Farace, Extensive modulation of a set of microRNAs in primary glioblastoma. Biochem Biophys Res Commun 334, 1351-1358 (2005).
12. R. El Fatimy, S. Subramanian, E. J. Uhlmann, A. M. Krichevsky, Genome Editing Reveals Glioblastoma Addiction to MicroRNA-10b. Mol Ther 25, 368-378 (2017).
13. G. Gabriely, M. Yi, R. S. Narayan, J. M. Niers, T. Wurdinger, J. Imitola, K. L. Ligon, S. Kesari, C. Esau, R. M. Stephens, B. A. Tannous, A. M. Krichevsky, Human glioma growth is controlled by microRNA-10b. Cancer Res 71, 3563-3572 (2011).
14. F. Guessous, M. Alvarado-Velez, L. Marcinkiewicz, Y. Zhang, J. Kim, S. Heister, B. Kefas, J. Godlewski, D. Schiff, B. Purow, Oncogenic effects of miR-10b in glioblastoma stem cells. Journal of neuro-oncology 112, 153-163 (2013).
15. C. Ma, F. Wei, H. Xia, H. Liu, X. Dong, Y. Zhang, Q. Luo, Y. Liu, Y. Li, MicroRNA-10b mediates TGF-beta1-regulated glioblastoma proliferation, migration and epithelial-mesenchymal transition. Int J Oncol 50, 1739-1748 (2017).
16. J. A. Chan, A. M. Krichevsky, K. S. Kosik, MicroRNA-21 is an antiapoptotic factor in human glioblastoma cells. Cancer Res 65, 6029-6033 (2005).
17. G. Gabriely, T. Wurdinger, S. Kesari, C. C. Esau, J. Burchard, P. S. Linsley, A. M. Krichevsky, MicroRNA 21 promotes glioma invasion by targeting matrix metalloproteinase regulators. Mol Cell Biol 28, 5369-5380 (2008).
18. C. Li, J. Sun, Q. Xiang, Y. Liang, N. Zhao, Z. Zhang, Q. Liu, Y. Cui, Prognostic role of microRNA-21 expression in gliomas: a meta-analysis. J Neurooncol 130, 11-17 (2016).
19. R. Shi, P. Y. Wang, X. Y. Li, J. X. Chen, Y. Li, X. Z. Zhang, C. G. Zhang, T. Jiang, W. B. Li, W. Ding, S. J. Cheng, Exosomal levels of miRNA-21 from cerebrospinal fluids associated with poor prognosis and tumor recurrence of glioma patients. Oncotarget 6, 26971-26981 (2015).
20. C. H. Yang, J. Yue, S. R. Pfeffer, M. Fan, E. Paulus, A. Hosni-Ahmed, M. Sims, S. Qayyum, A. M. Davidoff, C. R. Handorf, L. M. Pfeffer, MicroRNA-21 promotes glioblastoma tumorigenesis by down-regulating insulin-like growth factor-binding protein-3 (IGFBP3). J Biol Chem 289, 25079-25087 (2014).
21. N. M. Teplyuk, E. J. Uhlmann, G. Gabriely, N. Volfovsky, Y. Wang, J. Teng, P. Karmali, E. Marcusson, M. Peter, A. Mohan, Y. Kraytsberg, R. Cialic, E. A. Chiocca, J. Godlewski, B. Tannous, A. M. Krichevsky, Therapeutic potential of targeting microRNA-10b in established intracranial glioblastoma: first steps toward the clinic. EMBO Mol Med 8, 268-287 (2016).
22. I. Ben-Porath, M. W. Thomson, V. J. Carey, R. Ge, G. W. Bell, A. Regev, R. A. Weinberg, An embryonic stem cell-like gene expression signature in poorly differentiated aggressive human tumors. Nat Genet 40, 499-507 (2008).
23. P. Sathyan, P. O. Zinn, A. L. Marisetty, B. Liu, M. M. Kamal, S. K. Singh, P. Bady, L. Lu, K. M. Wani, B. L. Veo, J. Gumin, D. H. Kassem, F. Robinson, C. Weng, V. Baladandayuthapani, D. Suki, H. Colman, K. P. Bhat, E. P. Sulman, K. Aldape, R. R. Colen, R. G. Verhaak, Z. Lu, G. N. Fuller, S. Huang, F. F. Lang, R. Sawaya, M. Hegi, S. Majumder, Mir-21-Sox2 Axis Delineates Glioblastoma Subtypes with Prognostic Impact. J Neurosci 35, 15097-15112 (2015).
24. A. Belter, K. Rolle, M. Piwecka, A. Fedoruk-Wyszomirska, M. Z. Naskret-Barciszewska, J. Barciszewski, Inhibition of miR-21 in glioma cells using catalytic nucleic acids. Sci Rep 6, 24516 (2016).
25. M. F. Corsten, R. Miranda, R. Kasmieh, A. M. Krichevsky, R. Weissleder, K. Shah, MicroRNA-21 knockdown disrupts glioma growth in vivo and displays synergistic cytotoxicity with neural precursor cell delivered S-TRAIL in human gliomas. Cancer Res 67, 8994-9000 (2007).
26. Y. E. Seo, H. W. Suh, R. Bahal, A. Josowitz, J. Zhang, E. Song, J. Cui, S. Noorbakhsh, C. Jackson, T. Bu, A. Piotrowski-Daspit, R. Bindra, W. M. Saltzman, Nanoparticle-mediated intratumoral inhibition of miR-21 for improved survival in glioblastoma. Biomaterials 201, 87-98 (2019).
27. L. Shi, J. Chen, J. Yang, T. Pan, S. Zhang, Z. Wang, MiR-21 protected human glioblastoma U87MG cells from chemotherapeutic drug temozolomide induced apoptosis by decreasing Bax/Bcl-2 ratio and caspase-3 activity. Brain Res 1352, 255-264 (2010).
28. S. T. Wong, X. Q. Zhang, J. T. Zhuang, H. L. Chan, C. H. Li, G. K. Leung, MicroRNA-21 inhibition enhances in vitro chemosensitivity of temozolomide-resistant glioblastoma cells. Anticancer Res 32, 2835-2841 (2012).
29. Y. Ren, X. Zhou, M. Mei, X. B. Yuan, L. Han, G. X. Wang, Z. F. Jia, P. Xu, P. Y. Pu, C. S. Kang, MicroRNA-21 inhibitor sensitizes human glioblastoma cells U251 (PTEN-mutant) and LN229 (PTEN-wild type) to taxol. BMC Cancer 10, 27 (2010).
30. P. E. Nielsen, M. Egholm, R. H. Berg, O. Buchardt, Sequence-selective recognition of DNA by strand displacement with a thymine-substituted polyamide. Science 254, 1497-1500 (1991).
31. V. V. Demidov, V. N. Potaman, M. D. Frank-Kamenetskii, M. Egholm, O. Buchard, S. H. Sonnichsen, P. E. Nielsen, Stability of peptide nucleic acids in human serum and cellular extracts. Biochem Pharmacol 48, 1310-1313 (1994).
32. M. Egholm, O. Buchardt, L. Christensen, C. Behrens, S. M. Freier, D. A. Driver, R. H. Berg, S. K. Kim, B. Norden, P. E. Nielsen, PNA hybridizes to complementary oligonucleotides obeying the Watson-Crick hydrogen-bonding rules. Nature 365, 566-568 (1993).
33. I. A. Babar, C. J. Cheng, C. J. Booth, X. Liang, J. B. Weidhaas, W. M. Saltzman, F. J. Slack, Nanoparticle-based therapy in an in vivo microRNA-155 (miR-155)-dependent mouse model of lymphoma. Proc Natl Acad Sci USA 109, E1695-1704 (2012).
34. C. J. Cheng, R. Bahal, I. A. Babar, Z. Pincus, F. Barrera, C. Liu, A. Svoronos, D. T. Braddock, P. M. Glazer, D. M. Engelman, W. M. Saltzman, F. J. Slack, MicroRNA silencing for cancer therapy targeted to the tumour microenvironment. Nature 518, 107-110 (2015).
35. A. Tahmasbi Rad, S. Malik, L. Yang, T. K. Oberoi-Khanuja, M. P. Nieh, R. Bahal, A universal discoidal nanoplatform for the intracellular delivery of PNAs. Nanoscale 11, 12517-12529 (2019).
36. D. P. Bartel, MicroRNAs: target recognition and regulatory functions. Cell 136, 215-233 (2009).
37. S. Obad, C. O. dos Santos, A. Petri, M. Heidenblad, O. Broom, C. Ruse, C. Fu, M. Lindow, J. Stenvang, E. M. Straarup, H. F. Hansen, T. Koch, D. Pappin, G. J. Hannon, S. Kauppinen, Silencing of microRNA families by seed-targeting tiny LNAs. Nat Genet 43, 371-378 (2011).
38. A. Dragulescu-Andrasi, S. Rapireddy, B. M. Frezza, C. Gayathri, R. R. Gil, D. H. Ly, A simple gamma-backbone modification preorganizes peptide nucleic acid into a helical structure. J Am Chem Soc 128, 10258-10267 (2006).
39. G. He, S. Rapireddy, R. Bahal, B. Sahu, D. H. Ly, Strand invasion of extended, mixed-sequence B-DNA by gammaPNAs. J Am Chem Soc 131, 12088-12090 (2009).
40. B. Sahu, I. Sacui, S. Rapireddy, K. J. Zanotti, R. Bahal, B. A. Armitage, D. H. Ly, Synthesis and characterization of conformationally preorganized, (R)-diethylene glycol-containing gamma-peptide nucleic acids with superior hybridization properties and water solubility. J Org Chem 76, 5614-5627 (2011).
41. V. Rottiers, S. Obad, A. Petri, R. McGarrah, M. W. Lindholm, J. C. Black, S. Sinha, R. J. Goody, M. S. Lawrence, A. S. deLemos, H. F. Hansen, S. Whittaker, S. Henry, R. Brookes, S. H. Najafi-Shoushtari, R. T. Chung, J. R. Whetstine, R. E. Gerszten, S. Kauppinen, A. M. Naar, Pharmacological inhibition of a microRNA family in nonhuman primates by a seed-targeting 8-mer antimiR. Sci Transl Med 5, 212ra162 (2013).
42. M. Li, T. Zengeya, E. Rozners, Short peptide nucleic acids bind strongly to homopurine tract of double helical RNA at pH 5.5. J Am Chem Soc 132, 8676-8681 (2010).
43. B. G. Garchow, O. Bartulos Encinas, Y. T. Leung, P. Y. Tsao, R. A. Eisenberg, R. Caricchio, S. Obad, A. Petri, S. Kauppinen, M. Kiriakidou, Silencing of microRNA-21 in vivo ameliorates autoimmune splenomegaly in lupus mice. EMBO Mol Med 3, 605-615 (2011).
44. Y. E. Seo, T. Bu, W. M. Saltzman, Nanomaterials for convection-enhanced delivery of agents to treat brain tumors. Curr Opin Biomed Eng 4, 1-12 (2017).
45. Y. Deng, J. K. Saucier-Sawyer, C. J. Hoimes, J. Zhang, Y. E. Seo, J. W. Andrejecsk, W. M. Saltzman, The effect of hyperbranched polyglycerol coatings on drug delivery using degradable polymer nanoparticles. Biomaterials 35, 6595-6602 (2014).
46. Y. Deng, A. Ediriwickrema, F. Yang, J. Lewis, M. Girardi, W. M. Saltzman, A sunblock based on bioadhesive nanoparticles. Nat Mater 14, 1278-1285 (2015).
47. Y. Deng, F. Yang, E. Cocco, E. Song, J. Zhang, J. Cui, M. Mohideen, S. Bellone, A. D. Santin, W. M. Saltzman, Improved i.p. drug delivery with bioadhesive nanoparticles. Proc Natl Acad Sci USA 113, 11453-11458 (2016).
48. J. K. Hu, H. W. Suh, M. Qureshi, J. M. Lewis, S. Yaqoob, Z. M. Moscato, S. Griff, A. K. Lee, E. S. Yin, W. M. Saltzman, M. Girardi, Nonsurgical treatment of skin cancer with local delivery of bioadhesive nanoparticles. Proc Natl Acad Sci USA 118, (2021).
49. H. W. Suh, J. Lewis, L. Fong, J. Y. Ramseier, K. Carlson, Z. H. Peng, E. S. Yin, W. M. Saltzman, M. Girardi, Biodegradable bioadhesive nanoparticle incorporation of broad-spectrum organic sunscreen agents. Bioeng Transl Med 4, 129-140 (2019).
50. E. Song, A. Gaudin, A. R. King, Y. E. Seo, H. W. Suh, Y. Deng, J. Cui, G. T. Tietjen, A. Huttner, W. M. Saltzman, Surface chemistry governs cellular tropism of nanoparticles in the brain. Nat Commun 8, 15322 (2017).
51. Y.-E. Seo, H.-W. Suh, R. Bahal, A. Josowitz, J. Zhang, E. Song, J. Cui, S. Noorbakhsh, C. Jackson, T. Bu, Nanoparticle-mediated intratumoral inhibition of miR-21 for improved survival in glioblastoma. Biomaterials 201, 87-98 (2019).
52. L. Christensen, R. Fitzpatrick, B. Gildea, K. H. Petersen, H. F. Hansen, T. Koch, M. Egholm, O. Buchardt, P. E. Nielsen, J. Coull, R. H. Berg, Solid-phase synthesis of peptide nucleic acids. J Pept Sci 1, 175-183 (1995).
53. S. Malik, J. Lim, F. J. Slack, D. T. Braddock, R. Bahal, Next generation miRNA inhibition using short anti-seed PNAs encapsulated in PLGA nanoparticles. J Control Release 327, 406-419 (2020).
54. S. Malik, F. J. Slack, R. Bahal, Formulation of PLGA nanoparticles containing short cationic peptide nucleic acids. MethodsX 7, 101115 (2020).
55. Y.-R. Lee, M. Chen, P. P. Pandolfi, The functions and regulation of the PTEN tumour suppressor: new modes and prospects. Nature Reviews Molecular Cell Biology 19, 547-562 (2018).
56. F. Meng, R. Henson, H. Wehbe-Janek, K. Ghoshal, S. T. Jacob, T. Patel, MicroRNA-21 regulates expression of the PTEN tumor suppressor gene in human hepatocellular cancer. Gastroenterology 133, 647-658 (2007).
57. M. Mizoguchi, Y. Guan, K. Yoshimoto, N. Hata, T. Amano, A. Nakamizo, T. Sasaki, Clinical implications of microRNAs in human glioblastoma. Frontiers in oncology 3, 19 (2013).
58. Q. T. Ostrom, H. Gittleman, J. Fulop, M. Liu, R. Blanda, C. Kromer, Y. Wolinsky, C. Kruchko, J. S. Barnholtz-Sloan, CBTRUS statistical report: primary brain and central nervous system tumors diagnosed in the United States in 2008-2012. Neuro-oncology 17, iv1-iv62 (2015).
59. B. M. Alexander, T. F. Cloughesy, Adult glioblastoma. Journal of Clinical Oncology 35, 2402-2409 (2017).
60. J. Wang, E. Cazzato, E. Ladewig, V. Frattini, D. I. Rosenbloom, S. Zairis, F. Abate, Z. Liu, O. Elliott, Y.-J. Shin, Clonal evolution of glioblastoma under therapy. Nature genetics 48, 768 (2016).
61. M. Lim, Y. Xia, C. Bettegowda, M. Weller, Current state of immunotherapy for glioblastoma. Nature reviews Clinical oncology 15, 422-442 (2018).
62. C. W. Brennan, R. G. Verhaak, A. McKenna, B. Campos, H. Noushmehr, S. R. Salama, S. Zheng, D. Chakravarty, J. Z. Sanborn, S. H. Berman, The somatic genomic landscape of glioblastoma. Cell 155, 462-477 (2013).
63. M. Petersen, J. Wengel, LNA: a versatile tool for therapeutics and genomics. Trends Biotechnol 21, 74-81 (2003).
64. G. Zhang, L. Q. Jin, J. Hu, W. Rodemer, M. E. Selzer, Antisense Morpholino Oligonucleotides Reduce Neurofilament Synthesis and Inhibit Axon Regeneration in Lamprey Reticulospinal Neurons. PLoS One 10, e0137670 (2015).
65. J. Saarbach, P. M. Sabale, N. Winssinger, Peptide nucleic acid (PNA) and its applications in chemical biology, diagnostics, and therapeutics. Current opinion in chemical biology 52, 112-124 (2019).
66. C. J. Cheng, R. Bahal, I. A. Babar, Z. Pincus, F. Barrera, C. Liu, A. Svoronos, D. T. Braddock, P. M. Glazer, D. M. Engelman, MicroRNA silencing for cancer therapy targeted to the tumour microenvironment. Nature 518, 107-110 (2015).
67. L. L. Muldoon, C. Soussain, K. Jahnke, C. Johanson, T. Siegal, Q. R. Smith, W. A. Hall, K. Hynynen, P. D. Senter, D. M. Peereboom, E. A. Neuwelt, Chemotherapy delivery issues in central nervous system malignancy: a reality check. J Clin Oncol 25, 2295-2305 (2007).
68. I. T. Papademetriou, T. Porter, Promising approaches to circumvent the blood-brain barrier: progress, pitfalls and clinical prospects in brain cancer. Ther Deliv 6, 989-1016 (2015).
69. R. H. Bobo, D. W. Laske, A. Akbasak, P. F. Morrison, R. L. Dedrick, E. H. Oldfield, Convection-enhanced delivery of macromolecules in the brain. Proc Natl Acad Sci USA 91, 2076-2080 (1994).
70. M. A. Vogelbaum, M. K. Aghi, Convection-enhanced delivery for the treatment of glioblastoma. Neuro Oncol 17 Suppl 2, ii3-ii8 (2015).
71. J. N. Bruce, R. L. Fine, P. Canoll, J. Yun, B. C. Kennedy, S. S. Rosenfeld, S. A. Sands, K. Surapaneni, R. Lai, C. L. Yanes, E. Bagiella, R. L. DeLaPaz, Regression of recurrent malignant gliomas with convection-enhanced delivery of topotecan. Neurosurgery 69, 1272-1279; discussion 1279-1280 (2011).
72. U. Bogdahn, P. Hau, G. Stockhammer, N. K. Venkataramana, A. K. Mahapatra, A. Suri, A. Balasubramaniam, S. Nair, V. Oliushine, V. Parfenov, I. Poverennova, M. Zaaroor, P. Jachimczak, S. Ludwig, S. Schmaus, H. Heinrichs, K. H. Schlingensiepen, G. Trabedersen Glioma Study, Targeted therapy for high-grade glioma with the TGF-beta2 inhibitor trabedersen: results of a randomized and controlled phase IIb study. Neuro Oncol 13, 132-142 (2011).
73. J. Voges, R. Reszka, A. Gossmann, C. Dittmar, R. Richter, G. Garlip, L. Kracht, H. H. Coenen, V. Sturm, K. Wienhard, W. D. Heiss, A. H. Jacobs, Imaging-guided convection-enhanced delivery and gene therapy of glioblastoma. Ann Neurol 54, 479-487 (2003).
74. G. M. Bernal, M. J. LaRiviere, N. Mansour, P. Pytel, K. E. Cahill, D. J. Voce, S. Kang, R. Spretz, U. Welp, S. E. Noriega, L. Nunez, G. F. Larsen, R. R. Weichselbaum, B. Yamini, Convection-enhanced delivery and in vivo imaging of polymeric nanoparticles for the treatment of malignant glioma. Nanomedicine 10, 149-157 (2014).
75. N. U. Barua, M. Woolley, A. S. Bienemann, D. Johnson, M. J. Wyatt, C. Irving, O. Lewis, E. Castrique, S. S. Gill, Convection-enhanced delivery of AAV2 in white matter—a novel method for gene delivery to cerebral cortex. J Neurosci Methods 220, 1-8 (2013).
76. E. Allard, C. Passirani, J. P. Benoit, Convection-enhanced delivery of nanocarriers for the treatment of brain tumors. Biomaterials 30, 2302-2318 (2009).
77. J. K. Saucier-Sawyer, Y. E. Seo, A. Gaudin, E. Quijano, E. Song, A. J. Sawyer, Y. Deng, A. Huttner, W. M. Saltzman, Distribution of polymer nanoparticles by convection-enhanced delivery to brain tumors. J Control Release 232, 103-112 (2016).
78. C. G. Dong, W. K. Wu, S. Y. Feng, X. J. Wang, J. F. Shao, J. Qiao, Co-inhibition of microRNA-10b and microRNA-21 exerts synergistic inhibition on the proliferation and invasion of human glioma cells. International journal of oncology 41, 1005-1012 (2012).
79. H. Gibori, S. Eliyahu, A. Krivitsky, D. Ben-Shushan, Y. Epshtein, G. Tiram, R. Blau, P. Ofek, J. S. Lee, E. Ruppin, L. Landsman, I. Barshack, T. Golan, E. Merquiol, G. Blum, R. Satchi-Fainaro, Amphiphilic nanocarrier-induced modulation of PLK1 and miR-34a leads to improved therapeutic response in pancreatic cancer. Nat Commun 9, 16 (2018).
80. E. Jonsson, H. Fridborg, P. Nygren, R. Larsson, Synergistic interactions of combinations of topotecan with standard drugs in primary cultures of human tumor cells from patients. Eur J Clin Pharmacol 54, 509-514 (1998).
81. P. P. Wang, J. Frazier, H. Brem, Local drug delivery to the brain. Advanced drug delivery reviews 54, 987-1013 (2002).
82. A. J. Sawyer, J. M. Piepmeier, W. M. Saltzman, Cancer issue: new methods for direct delivery of chemotherapy for treating brain tumors. The Yale journal of biology and medicine 79, 141 (2006).
83. M. I. Love, W. Huber, S. Anders, Moderated estimation of fold change and dispersion for RNA-seq data with DESeq2. Genome Biol 15, 550 (2014).
84. G. Yu, L. G. Wang, Y. Han, Q. Y. He, clusterProfiler: an R package for comparing biological themes among gene clusters. OMICS 16, 284-287 (2012).
85. H. Y. Huang, Y. C. Lin, J. Li, K. Y. Huang, S. Shrestha, H. C. Hong, Y. Tang, Y. G. Chen, C. N. Jin, Y. Yu, J. T. Xu, Y. M. Li, X. X. Cai, Z. Y. Zhou, X. H. Chen, Y. Y. Pei, L. Hu, J. J. Su, S. D. Cui, F. Wang, Y. Y. Xie, S. Y. Ding, M. F. Luo, C. H. Chou, N. W. Chang, K. W. Chen, Y. H. Cheng, X. H. Wan, W. L. Hsu, T. Y. Lee, F. X. Wei, H. D. Huang, miRTarBase 2020: updates to the experimentally validated microRNA-target interaction database. Nucleic Acids Res 48, D148-D154 (2020).

1. A composition comprising a peptide nucleic acid, wherein the peptide nucleic acid is a cationic gamma-(γ)-modified peptide nucleic acid.

2. The composition of claim 1, wherein
the peptide nucleic acid comprises a serine modification at the γ-position;
the peptide nucleic acid comprises at least one, two or three arginine residues on the N-terminus;
the peptide nucleic acid comprises at least one, two or three arginine residues on the C-terminus; and/or
the peptide nucleic acid comprises a nucleotide sequence targeting a seed region of an oncomiR.

3-5. (canceled)

6. The composition of claim 1, wherein the peptide nucleic acid comprises from N-terminus to C-terminus: three arginine residues, a nucleotide sequence targeting a seed region of an oncomiR, and one arginine at the C-terminus.

7. The composition of claim 2, wherein the oncomiR is selected from the group consisting of miR-21 and miR-10b.

8. The composition of claim 1, wherein
the composition comprises a peptide nucleic acid comprising a nucleotide sequence targeting the seed region of miR-21; and/or
the composition comprises a peptide nucleic acid comprising a nucleotide sequence targeting the seed region of miR-10b.

9. (canceled)

10. (canceled)

11. The composition of claim 2, wherein the nucleotide sequence targeting the seed region of the oncomiR comprises about 5, 6, 7, or 8 nucleotides in length.

12. (canceled)

13. The composition of claim 8, wherein
the nucleotide sequence targeting the seed region of miR-21 comprises GATAAGCT; and/or
the nucleotide sequence targeting the seed region of miR-10b comprises TACAGGGT.

14. (canceled)

15. A nanoparticle comprising the peptide nucleic acid of claim 1, wherein the peptide nucleic acid is encapsulated within the nanoparticle, and wherein the peptide nucleic acid is a cationic gamma-(γ)-modified peptide nucleic acid.

16. The nanoparticle of claim 15, wherein
the nanoparticle comprises a poly-lactic acid and hyperbranched polyglycerol (PLA-HPG) polymer;
the nanoparticle is modified with aldehyde groups; and/or
the nanoparticle is bioadhesive.

17. (canceled)

18. (canceled)

19. The nanoparticle of claim 15, comprising a first peptide nucleic acid comprising a nucleotide sequence targeting a seed region of miR-21, and a second peptide nucleic acid comprising a nucleotide sequence targeting a seed region of miR-10b, wherein the first and the second peptide nucleic acids are encapsulated within the nanoparticle, wherein the first and the second peptide nucleic acid are each cationic gamma-(γ)-modified peptide nucleic acids.

20. A pharmaceutical composition comprising the composition of claim 1, and a pharmaceutically acceptable excipient.

21. The pharmaceutical composition of claim 20, comprising a first nanoparticle comprising a first peptide nucleic acid comprising a nucleotide sequence targeting a seed region of miR-21 and a second nanoparticle comprising a second peptide nucleic acid comprising a nucleotide sequence targeting a seed region of miR-10b.

22. A method of treating a disease in a subject in need thereof, comprising administering to the subject a therapeutically effective amount of the composition of claim 1, thereby treating the disease in the subject in need thereof.

23. The method of claim 22, where the disease is cancer.

24. The method of claim 22 or 23, wherein the cancer is glioblastoma.

25. A method of reducing a tumor growth in a subject in need thereof, comprising administering to the subject a therapeutically effective amount of the composition of claim 1, thereby reducing the tumor growth in the subject in need thereof.

26. A method of prolonging survival time of a subject in need thereof, of increasing sensitivity to a chemotherapy in a subject in need thereof, or of increasing apoptosis of tumor cells in a subject in need thereof, comprising administering to the subject a therapeutically effective amount of the composition of claim 1, thereby prolonging survival time of the subject in need thereof, increasing sensitivity to a chemotherapy in a subject in need thereof, or increasing apoptosis of tumor cells in a subject in need thereof.

27. (canceled)

28. (canceled)

29. The method of claim 22, wherein
the method results in a decrease in miR-10b and/or miR21 levels, and/or
the method results in a decrease in VEGFA and ITGB8 levels.

30. (canceled)

31. The method of claim 22, wherein the composition is administered to the subject by convection-enhanced drug delivery (CED).

32. (canceled)

33. The method of claim 22, further comprising administering to the subject an additional therapeutic agent, optionally, wherein the additional therapeutic agent is selected from the group consisting of a chemotherapeutic agent, a growth inhibitory agent, an anti-angiogenesis agent, an anti-neoplastic composition and a combination of any of the foregoing, optionally, wherein the chemotherapeutic agent is temozolomide.

34. (canceled)

35. (canceled)


SEQUENCE LISTING (4 sequences)

SEQ ID NO: 1 - RNA, 22 nt, synthetic construct
  misc_feature 1..22: note = Synthetic: OncomiR-21
  source 1..22: mol_type = other RNA
tagcttatcagactgatgttga

SEQ ID NO: 2 - RNA, 23 nt, synthetic construct
  misc_feature 1..23: note = Synthetic: OncomiR-10b
  source 1..23: mol_type = other RNA
taccctgtagaaccgaatttgtg

SEQ ID NO: 3 - DNA, 22 bp, synthetic construct
  misc_feature 1..22: note = Synthetic: PNA-21
  modified_base 1: mod_base = OTHER; note = TAMRA-OOO: 5-carboxytetramethylrhodamine (TAMRA) and 8-amino-2,6,10-trioxaoctanoic acid (OOO)
  modified_base 22: mod_base = OTHER; note = arginine (R) appended to the C-terminus
  source 1..22: mol_type = other DNA
tcaacatcagtctgataagcta

SEQ ID NO: 4 - DNA, 23 bp, synthetic construct
  misc_feature 1..23: note = Synthetic: PNA-10b
  modified_base 1: mod_base = OTHER; note = TAMRA-OOO: 5-carboxytetramethylrhodamine (TAMRA) and 8-amino-2,6,10-trioxaoctanoic acid (OOO)
  modified_base 23: mod_base = OTHER; note = arginine (R) appended to the C-terminus
  source 1..23: mol_type = other DNA
cacaaattcggttctacagggta